(19) United States
(12) Patent Application Publication
(10) Pub. No.: US 2024/0033354 A1
(43) Pub. Date: Feb. 1, 2024

(54) A BROADLY NEUTRALIZING MOLECULE AGAINST CLOSTRIDIUM DIFFICILE TOXIN B

(71) Applicant: THE REGENTS OF THE UNIVERSITY OF CALIFORNIA, Oakland, CA (US)

(72) Inventors: Rongsheng Jin, Irvine, CA (US); Peng Chen, Irvine, CA (US)

(21) Appl. No.: 18/043,878

(22) PCT Filed: Sep. 2, 2021
(86) PCT No.: PCT/US21/48921

§ 371 Date: Mar. 2, 2023

Related U.S. Application Data
(60) Provisional application No. 63/073,831, filed on Sep. 2, 2020.

(51) Int. Cl. A61K 39/40; C07K 14/705; C07K 16/40; A61K 38/14; A61K 31/4164; A61K 31/7048; A61P 31/04

(52) U.S. Cl. CPC A61K 39/40; C07K 14/705; C07K 16/40; A61K 38/14; A61K 31/4164; A61K 31/7048; A61P 31/04; C07K 2319/30; C07K 2317/569

(57) ABSTRACT

The present invention has designed and produced a family of recombinant proteins that could provide broad-spectrum protection/neutralization against most subtypes of TcdB, and therefore could be developed into therapies against GDI. The designs of these novel proteins are based on the first 3D structure of TcdB1 in complex with its receptor CSPG4 that was recently determined. The present invention demonstrates that these newly designed proteins are more potent and provide broader-spectrum protection than the commercial antibody bezlotoxumab in terms of neutralizing diverse subtypes of TcdB.

### CROSS-REFERENCES TO RELATED APPLICATIONS

This application claims benefit of U.S. Provisional Application No. 63/073,831 filed Sep. 2, 2020, the specification of which is incorporated herein in its entirety by reference.

### STATEMENT REGARDING FEDERALLY SPONSORED RESEARCH OR DEVELOPMENT

This invention was made with government support under Grant Nos. ROI AI139087 and R01 AI125704 awarded by NIH. The government has certain rights in the invention.

### REFERENCE TO A SEQUENCE LISTING

Applicant asserts that the information recorded in the form of an Annex C/ST.25 text file submitted under Rule 13ter.1(a), entitled UCI_20_24_PCT_Sequencing_Listing_ST25, is identical to that forming part of the international application as filed. The content of the sequence listing is incorporated herein by reference in its entirety

### FIELD OF THE INVENTION

The present invention features a neutralizing receptor decoy antibody (RDA) for the prevention and treatment of Clostridium difficile infection (CDI) caused by a C. difficile toxin.

### BACKGROUND OF THE INVENTION

Clostridicides difficile (formerly Clostridium difficile, or C. difficile) is a Gram-positive, spore-forming anaerobic bacterium. With estimated ˜223,900 infections, 12,800 deaths, and $1 billion healthcare cost in the US in 2017, C. difficile infection (CDI) is the most frequent cause of healthcare-acquired gastrointestinal infections and death in developed countries. There is also an increasing frequency of community-associated infections in recent years. Two homologous C. difficile exotoxins, toxin A (TcdA) and toxin B (TcdB), are the major virulence factors. Among them, TcdB alone is capable of causing the full-spectrum of diseases associated with CDI in humans, and pathogenic TcdA⁻TcdB⁺ strains have been routinely isolated in clinics. The key role of TcdB in CDI is further confirmed by the finding that an FDA-approved anti-TcdB monoclonal antibody (bezlotoxumab) reduced CDI recurrence in humans.

The current standard of care for CDI consists of administration of antibiotics such as vancomycin or fidaxomicin that target the bacterium but also perpetuate gut microbiome, often leading to disease recurrence (up to 35%). A monoclonal antitoxin antibody, ZINPLAVA™ (bezlotoxumab) from Merck, was approved by FDA to reduce recurrence of CDI in patients who are receiving antibacterial drug treatment of CDI and are at high risk for CDI recurrence. ZINPLAVA™ is not indicated for the treatment of CDI. No other drug or vaccine for CDI is currently available.

However, TcdB has greatly diversified throughout its entire primary sequence up to 11% during evolution. For example, many hypervirulent fluoroquinolone-resistant lineages such as BI/NAP1/027 strains, which emerged in North America with major outbreaks in early 2000's, express a variant of TcdB (designated TcdB2) that is ˜8% sequence variation from the endemic TcdB (designated TcdB1). The sequence variations have impacts on TcdB activity and pathogenicity as evidenced by the observations that bezlotoxumab showed ˜200-fold lower potency on neutralizing TcdB2 than TcdB1. Therefore, the complexity of TcdB variation has posed significant challenges for developing effective therapeutic antibodies, vaccines, and diagnostic assays with sufficient broadness.

Here, the present invention has determined the cryogenic electron microscopy (cryo-EM) structure of TcdB1 binding to a host receptor and has identified a unique interface in TcdB, which involves residues scattering across multiple TcdB domains including its CPD. These residues are highly conserved across most TcdB variants known to date. Additionally, the present invention has determined a rationally designed mimicking decoy antibody that inhibits both TcdB1 and TcdB, suggesting a strategy for broad-spectrum therapeutics against TcdB.

### BRIEF SUMMARY OF THE INVENTION

It is an objective of the present invention to provide for a neutralizing receptor decoy antibody (RDA) composition that allows for treatment or prevention of Clostridium difficile infection (CDI) caused by a protein toxin produced by C. difficile (e.g., TcdB), as specified in the independent claims. Embodiments of the invention are given in the dependent claims. Embodiments of the present invention can be freely combined with each other if they are not mutually exclusive.

TcdB is more virulent than TcdA and more important for inducing the host inflammatory and innate immune response. TcdB (˜270 kDa) is composed of four structural modules: a N-terminal glucosyltransferase domain (GTD), followed by a cysteine protease domain (CPD), an intermingled membrane translocation delivery domain and receptor-binding domain (DRBD), and a large C-terminal combined repetitive oligopeptides domain (CROPs). It is well accepted that the DRBD and CROPs are responsible for receptor recognition, and the two enzymatic domains GTD and CPD are delivered to the cytosol where the GTD glucosylates small GTPases of the Rho family, leading to actin cytoskeleton disruption and cell death. It is worth noting that a unique hinge region located between the DRBD and CROPs is essential for toxicity, which serves as a critical structural linchpin to mediate structural communications among all four domains of TcdB.

In addition to the complex structure of TcdB, it has been observed that TcdB variants may change their strategies to recognize host receptors for cell entry. The Wnt receptor frizzled proteins (FZDs) and chondroitin sulfate proteoglycan 4 (CSPG4, also known as NG2 in rodents) are two major candidate receptors for TcdB. CSPG4 is a single transmembrane domain protein conserved across evolution, with no apparent redundant isoforms in humans. Unlike FZDs that are expressed in the colonic epithelium, CSPG4 is highly expressed in many immature progenitor cells such as oligodendrocyte progenitor cells and mesenchymal stem cells. While its function remains to be fully established, it has been shown to promote cell proliferation, adhesion, migration, as well as mediate binding of many growth factors such as basic fibroblast growth factor (bFGF) and integrin. TcdB1 binds FZDs and CSPG4 simultaneously, indicating that FZDs and CSPG4 are recognized by distinct regions of TcdB. However, many clinically important TcdB variants, represented by TcdB2, bind CSPG4 but not FZDs, because they have residue substitutions in the FZD-binding site that abolish their binding to FZDs. Moreover, the therapeutic antibody bezlotoxumab reduces binding of TcdB1 to CSPG4 in vitro, suggesting CSPG4 may contribute to TcdB pathogenesis in humans, These findings suggest that CSPG4 could be a broad-spectrum receptor for diverse TcdB variants and a promising therapeutic target in CDI.

In some embodiments, the present invention may feature a broad-spectrum neutralizing composition comprising a neutralizing receptor decoy antibody (RDA) that neutralizes a toxin of Clostridium difficile in various strains. In other embodiments, the present invention may also feature a method of neutralizing a toxin of C. difficile. In some embodiments, the method comprises producing a neutralizing receptor decoy antibody (RDA) composition that binds to C. difficile toxin and blocks it from binding to cell surface receptors.

Additionally, in further embodiments, the present invention may feature a method of treating a Clostridium difficile infection (CDI) in a patient in need thereof. In some embodiments, the method comprises administering a standard of care (SOC) antibiotic and administering a therapeutically effective dose of a neutralizing receptor decoy antibody (RDA) composition.

Finally, in some embodiments, the present invention features a method of treating and/or preventing a Clostridium difficile infection (CDI) with a vaccine composed of the chondroitin sulfate proteoglycan 4 (CSPG4)-binding epitope on TcdB in a patient in need thereof. In some embodiments, the method comprises the steps of administering a CSPG4-binding epitope to a patient and eliciting an immune response. In some embodiments, the antibodies produced by the immune response bind to TcdB and prevent it from binding to CSPG4 for cell entry and thus provide protection to the patient.

One of the unique and inventive technical features of the present invention is the use of a neutralizing receptor decoy antibody (RDA) composition. Without wishing to limit the invention to any theory or mechanism, it is believed that the technical feature of the present invention advantageously provides for highly effective neutralization of various TcdB subtypes of the C. difficile that ultimately cause CDI. None of the presently known prior references or work has the unique inventive technical feature of the present invention.

Furthermore, the prior references teach away from the present invention. For example, the antibody bezlotoxumab currently being marketed by Merck is effective at inhibiting the C. difficile TcdB1 toxin but drastically less potent to inhibit TcdB2 and many other TcdB subtypes due to amino acid changes in the bezlotoxumab-binding epitopes.

Furthermore, the inventive technical features of the present invention contributed to a surprising result. For example, the present invention was able to determine the 3-dimensional structure of TcdB1 binding to the CSPG4 receptor and precisely determine the exact fragment (out of a total of 2,322 amino acids) of CSPG4 that sufficiently binds to TcdB1. Using the structural data, the present invention was able to generate a recombinant, highly expressed, stable, small fragment of CSPG4 as a receptor decoy that is able to prevent TcdB1 from binding to the full-length CSPG4 and therefore neutralize TcdB1 toxin. Furthermore, this CSPG4 decoy is effective against both TcdB1 and TcdB2 and most TcdB subtypes, because the CSPG4-binding site is conserved on TcdB1, TcdB2, and most known TcdB subtypes (see FIGS. 18A-18D).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows non-limiting designs of mono-, bi, and tri-specific receptor decoy antibody (RDA) composition comprising a fragment crystallizable region (Fc region) fragment, a fragment of a chondroitin sulfate proteoglycan 4 (CSPG4) receptor, a fragment of frizzled protein (FZD) receptor, a VHH nanobody, or a combination thereof.

FIGS. 2A, 2B, 2C, 2D, 2E, and 2F shows the overall structure of the TcdB-CSPG4 complex, FIG. 2A shows schematic diagrams showing the domain structures of TcdB and CSPG4, as well as the domain boundaries for TcdB^{core} and CSPG4^{mini} used for cryo-EM studies. GTD: glucosyltransferase domain; CPD: cysteine protease domain; DRBD: delivery and receptor-binding domain; CROPs: combined repetitive oligopeptides domain; Hinge: a key fragment between the DRBD and CROPs that mediates structural communications among all 4 domains of TcdB. CSPG4 is composed of two predicted laminin G domains, 15 CSPG repeats, a transmembrane domain (TM), and a cytosolic region. FIG. 2B shows the 3.17 Å resolution cryo-EM map of the TcdB^{core}-Repeat1 complex segmented, whereas Repeat1 is the TcdB-binding fragment of CSPG4. FIG. 2C shows a cartoon representation of the structure of the TcdB^{core}-Repeat1 complex that is shown in similar orientations as FIG. 2B. FIG. 2D shows the structure of Repeat1 of CSPG4 with the disulfide bond shown as sticks. FIG. 2E shows the structure of the TcdB^{core}-Repeat1 complex was superimposed to TcdB holotoxin (PDB: 6OQ5). The Repeat1-bound TcdB is colored (i.e., grey) and the unliganded TcdB is colored black with its CROPS II-IV omitted for clarity. The TcdB-bound Repeat1 is shown as a surface model. FIG. 2F shows that Repeat1 triggers local structural changes in the CPD and hinge of TcdB upon binding. For clarity, only residues 569-577 in the CPD and residues 1803-1812 in the hinge are shown in the context of Repeat1.

FIGS. 3A, 3B, and 3C shows cross-linking mass spectrometry (XL-MS) studies of the TcdB-CSPG4 complex. FIG. 3A shows an XL-MS analysis workflow for accurate identification of DHSO cross-linked peptides from 3 replicates (Rep1-3) of cross-linked TcdB-CSPG4 complex. FIG. 3B shows representative MSⁿ identification of a DHSO inter-linked peptide of the TcdB-CSPG4 complex. First, the cross-linked peptide [α-β]⁴⁺ (m/z 745.3986⁴⁺) was detected in MS¹. Next, it was selected for MS² analysis and yielded two characteristic fragment ion pairs, i.e. α_{A}/β_{T} (m/z 597.35²⁺/884.43²⁺) and α_{T}/β_{A} (m/z 613.34²⁺/868.44²⁺). Finally, MS³ analysis of α_{A} (m/z 597.35²⁺) identified its sequence as ⁶³⁶IPSIISD_{A}RPK⁶⁴⁶ (SEQ ID NO: 29), in which the aspartic acid residue at position 7 was modified with an alkene moiety. MS³ analysis of β_{T} (m/z 884.43²⁺) identified its sequence as ⁴⁵¹HVQPTLDLME_{T}AELR⁴⁶⁴ (SEQ ID NO: 30) in which the glutamic acid residue at position 10 was modified with unsaturated thiol moiety. Thus, the cross-link of TcdB:0642 to CSPG4:E460 was determined. FIG. 3C shows the illustrations of the identified inter-protein cross-links between CSPG4 and TcdB in the context of the full length proteins. It was noted that residue E92 of CSPG4 could be cross-linked to E760 and D1490 that are located in the CPD and DRBD of TcdB, respectively. These two residues are ˜97 Å away from each other on TcdB holotoxin, which cannot be simultaneously reached by E92 of CSPG4 via DHSO that has a distance limitation of ˜35 Å. This data suggests that the laminin G motifs of CSPG4 adopt flexible conformations and could transiently move within ˜35 Å of the CPD or DRBD of TcdB. The linkages between the flexible regions of CSFG4 and TcdB were shown as dashed lines.

FIGS. 4A, 4B, 4C, 4D, 4E, 4F, and 4G show Tcd8 recognizes CSFG4 using a composite binding site involving multiple domains. FIG. 4A shows the CSFG4 Repeat1 binds at a groove formed by the CPD, DRBD, hinge, and CROPS I. TcdB^{core} and Repeat1 are shown as a surface and a cartoon representation, respectively. FIGS. 4B and 2C shows an open-book view of the TcdBcore-Repeat1 interface. The amino acids in Repeat1 that constitute the three TcdB-binding subsites are colored green and outlined in boxes (FIG. 4C), while their detailed interactions with TcdB are further illustrated in FIGS. 4D, 4E, and 4F. FIGS. 4D, 4E, and 4F show close-up views of the TcdB-CSPG4 interface with interacting amino acids shown in stick models. FIG. 4G shows graphical representations of sequence conservation of CSPG4-binding residues in TcdB (SEQ ID NO: 31. For example, CSPG4 may be recognized by these conserved CSPG4-binding residues on TcdB variants, which include but not limit to residue number 563, 564, 567, 566, 573, 575, 602, 603, 621, 1754, 1758, 1509, 1811, 1812, 1816, 1818, 1819, 1823, 1825, 1831, 1850.). The height of symbols at each position indicates the relative frequency of each amino acid at that position based on analyses of 206 unique TcdB variants.

FIGS. 5A, 5B, 5C, 5D, 5E, 5F, 5G, and 5H shows biochemical characterization and workflow of cryo-EM reconstruction of the TcdB-CSPG4 complex. FIGS. 5A and 5B shows the quality of the TcdB^{core}-CSPG4^{mini} complex used for cryo-EM studies was characterized by SDS-PAGE and dynamic light scattering (DLS), a representative result from 3 similar results was reported. FIGS. 5C and 5D shows an example of a cryo-EM micrograph, the scale bar represents 83 Å (FIG. 5C) and 2D classes, the scale bar represents 120 Å (FIG. 5D) FIG. 5F shows an overview of the cryo-EM data processing and structure determination of the TcdB-CSPG4 complex using different box sizes. Overviews of reconstruction of the TcdB-CSPG4 complex are shown in the bottom panels. FIG. 5F shows the TcdB holotoxin (PDB: 6OQ5) was fitted to a 3.37 Å resolution EM map. FIG. 5G shows the gold-standard Fourier shell correlation (FSC) plots of 3D reconstruction of the 3.17 Å resolution map as calculated in cryoSPARC. FIG. 5H shows an angular distribution of particles included in the final cryo-EM reconstruction of the 3.17 Å resolution map.

FIGS. 6A and 6B show representative cryo-EM densities of the TcdB-CSPG4 complex at 3.17 Å resolution. Representative cryo-EM densities for TcdB (FIG. 6A) and CSPG4 (FIG. 6B).

FIGS. 7A and 7B show bio-layer interferometry (BLI) analyses of TcdB1 and TcdB2 binding to CSPG4 Repeat1-Fc. FIGS. 7A and 7B show representative binding curves with CSPG4 Repeat1-Fc as a ligand immobilized on anti-human IgG Fc capture (AHC) biosensors and TcdB1 or TcdB2 as the analytes. The concentrations of TcdB1 and TcdB2 examined were labeled in each panel. The shown binding analysis results are means ±s.d. from three independent experiments.

FIG. 8 shows TcdB variants adopt wild-type-like structures. The thermal stability of proteins was measured using a fluorescence-based thermal shift assay on a StepOne real-time PCR system (ThermoFisher). Protein melting was monitored using a hydrophobic dye, SYPRO Orange (Sigma-Aldrich), as the temperature was increased in a linear ramp from 25° C. to 95° C. The midpoint of the protein-melting curve (Tₘ) was determined using the software provided by the instrument manufacturer. The data are presented as means±s.d. (n=3). All the TcdB1 variants showed Tₘ values comparable to the wild-type protein, indicating correct protein folding.

FIGS. 9A, 9B, 9C and 9D show structure-based mutagenesis analyses of the interactions between Tcd8 and CSPG4. FIG. 9A shows the indicated TcdB mutants were tested for binding to cells. Purified WT and mutated TcdB (10 nM) were incubated with WT or CSPG4−/− HeLa cells. Cells were washed three-times by PBS, harvested, and cell lysates were analyzed by immunoblot detecting TcdB. Actin served as a loading control. FIG. 3B and 3C shows the sensitivity of CSPG4−/− FIG. 9B) and WT (FIG. 9C) HeLa cells to mutated TcdB were examined using the standard cytopathic cell-rounding assay. Error bars indicate mean±s.d. (n=3 biologically independent experiments). FIG. 9D shows the ratios of CR50 values on CSPG4−/− vs. WT HeLa cells from panels FIG. 9B and FIG. 9C were calculated and plotted, reflecting the fold-of-change in reduction of toxicity on CSPG4−/− cells compared with WT cells. n=3 for all groups. The upper and lower bounds of boxes indicate the maximum and minimum values of each group. The middle lines indicate the median values of each group. p-values by t-test: *:p≤0.05.

FIGS. 10A and 10B show the characterization of the interactions between TcdB and CSPG4 by structure-based mutagenesis. FIG. 10A shows the binding of TcdB1 variants to Repeat1-Fc immobilized on Protein A resins was examined using pull-down assays. FIG. 10B shows the binding of Repeat1-Fc variants to the Twin-strep tagged TcdB1 immobilized on Strep-Tactin resins was examined using pull-down assays. Samples were analyzed by SOS-PAGE and Coomassie Blue staining. The gels are representative of three independent experiments.

FIGS. 11A, 11B, 11C, 11D, 11E, 11F, 11G, 11H, 11I, 11J, 11K, 11L, and 11M show size-exclusion chromatography analysis of Repeat1-Fc and its variants. FIG. 11A-11M show representative elution profiles of Repeat1-Fc and its variants over a Superdex 200 Increase size-exclusion column, with the horizontal and vertical axes representing the elution volume and the normalized OD₂₈₀ absorbance, respectively. The peak elution volume for each protein is listed.

FIGS. 12A, 12B, 12C, 12D, 12E, 12F, and 12G show the analysis of C. difficile colonization and colon tissue damage in COI mouse models and cecum injection models. FIG. 12A shows a schematic diagram of the C. difficile infection model. WT and CSPG4^{-/-} mice were fed with antibiotic water for three days before resuming regular water for 24 h. A single dose of clindamycin (10 mg/kg) was administered to mice via intraperitoneal injection (i.p.). C. difficile spores (M7404, tcdA⁻) and mock (PBS) were administered to mice through oral gavage at 24 h after the injection. Mice were observed for another 48 h. FIG. 12B shows the WT and CSPG4^{-/-} mice were infected with 1×10⁵ C. difficile spores. Three groups of infection experiments were performed: mock to WT (n=4); M7404, tcdA⁻ to WT mice (n=8); and M7404, tcdA⁻ to CSPG4 mice (n=9). The weight loss of mice was recorded and shown. Error bars indicate mean±s.d., p-values by one-way ANOVA: ****: p≤0.0001, ***: p≤0.001, **: p≤0.01, *p≤0.05. The p-values of mock vs. C. difficile to WT, mock vs. C. difficile to CSPG4^{-/-}, and C. difficile to WT vs. C. difficile to CSPG4^{-/-} at 24 h are 0.0061, 0.0624, and 0.3314, at 48 h are 0.0001, 0.0383, and 0.0004. FIGS. 12C, 12D, and 120Dshow the WT (n=5) and CSPG4^{-/-} mice (n=5) were infected with 1×10⁴ C. difficile spores (M7404 tcdA⁻). Feces were collected at 24 h, 48 h, and 72 h, and dissolved in 50% ethanol. The dissolved feces were serial diluted, and the colony-forming unit (CFU) of C. diff spores/gram (g) of feces were quantified (FIG. 12C, lett panel), and the toxin titter (arbitrary unit/gram feces) was tested by the cytopathic effects (FIG. 12C, right panel). The arbitrary unit was defined as the dilution fold to reach CR₅₀. Error bars indicate mean±s.d., p-values by t test: ****: p≤0.0001, ***: p≤0.001 **: p≤0.01, *: p≤0.05. The p-values of 24 h, 48 h, and 7 2h for CFU are 0.831465, 0.671835, and 0.616704, for arbitrary toxins are 0.786909, 0.926407, and 0.628095. Cecum tissues were harvested at 90 h and subjected to H&E staining (scale bar represents 100 μm. M7404, tcdA⁻ to WT mice n=5, and M7404, tcdA⁻ to CSPG4^{-/-} mice n=5) (FIG. 12D) and histological analysis (FIG. 12E). Error bars indicate mean±s.d., p-values by t test; ****: p≤0.0001, ***: p≤0.001 **: p≤0.01, *: p≤0.05. The p-values of inflammation, hemorrhagic congestion, epithelial disruption, submucosal edema, and histological scores are 0.0005, 0.0005, 0.0144, 0.0003, and <0.0001. FIG. 12F shows Repeat1-Fc and CRD2 (preys) were pulled down by the Twin-strep-tagged TcdB1 (bait) immobilized on Strep-Tactin resins. Samples were analyzed by SOS-PAGE and Cooniassie Blue staining, and the gel is representative of three independent experiments. FIG. 12G shows the Claudin-3 intensity of immunostaining shown in FIG. 14C was quantified by ImageJ.

FIGS. 13A, 13B, 13C, and 13D shows CSPG4 is a physiological relevant cellular receptor for TcdB in vivo. FIG. 13A shows three groups of infection experiment were performed: mock to NT (n=4); M7404, tcdA− to WT mice (n=8): and M7404, tcdA− to CSPG4−/− mice (n=9). The representative cecum and colon of infected mice that were harvested at 48 h. FIGS. 13B, 4C, and 4D shows the harvested cecum was processed with hernatoxylin and eosin staining (scale bar represents 100 μm, mock n=4, C. difficile to WT n=4, C. difficile to CSPG4−/− n=5) (FIG. 13B), scored based on inflammatory cell infiltration, hemorrhagic congestion, epithelial disruption, and submucosal edema (FIG. 13C), and subjected to immunofluorescence staining by epithelial cell junction marker Claudin-3 (scale bar represents 50 μm, mock n=3, C. difficile to WT n=3, C. difficile to CSPG4−/− n=3) (FIG.40). In FIG. 13C, error bars indicate mean±SEM (mock n=4, C. difficile to WT n=4, C. difficile to CSPG4−/− n=5). P-values were calculated by post hoc analysis of a one-way ANOVA using Holm-Sidak's test for multiple comparisons: ****: p≤0.0001, ***: p≤0.001, **: p≤0.01, *: p≤0.05. Exact p values are presented in the accompanying source data.

FIGS. 14A, 14B, 14C, and 14D show mutations that selectively abolishing CSPG4 or FZD binding reduce toxicity of TcdB on cecum tissues. FIG. 14A shows a structural model of TcdB holotoxin with CSFG4 and FZD bound at two independent sites. The model is built based on superposition of the structures of TcdB1 holotoxin (PDB: 6OQ5), the TcdB-FZD complex (PDB: 6C0B), and the TcdB-CSPG4 complex (this work). FIGS. 14B, 5C, and 5D shows the indicated TcdB mutants or the control PBS was injected into the cecum of CD1 mice in vivo. The cecum tissues were harvested 6 h later and subjected to histological analysis with representative images (scale bars represent 100 μm, PBS n=4, TcdB n=5, TcdB^{GFE} n=5, and TcdB^{FZD-/CSP4-} n=5, TcdB^{CSPG4-} n=5) (FIG. 14B), immunostaining analysis for the tight junction marker Claudin-3 (scale bars represent 50 μm, PBS n=3, TcdB n=3, TcdB^{GFE} n=3, and TcdB^{FZD-/CSP4-} n=3, TcdB^{GSPG4-} n=3) (FIG. 14C), and pathological scores (error bars indicate mean±SEM, PBS n=4, TcdB n=5, TcdB^{GFE} n=5, and TcdB^{FZD-/GSPG4-} n=5, TcdB^{CSPG4-} n=5) (FIG. 14D). P-values were calculated through post hoc analysis of a one-way ANOVA using Holm-Sidak's test for multiple comparisons: ***: p≤0.001, ***: p≤0.001, **: 0.01, *: p≤0.05. Exact p values are presented in the accompanying source data.

FIGS. 15A, 15B, 15C, 15D, and 15E show bezlotoxumab competes with CSPG4 in an allosteric manner. FIG. 15A shows the crystal structure of a fragment of TcdB1 consisting of the CROPs I and II (residues 1833-2101) is shown as a surface model, while the epitope-1 and epitope-2 of bezlotoxumab are colored blue and purple, respectively (POE: 4NP4). FIG. 15B shows Bezlotoxumab blocks both TcdB1 and TcdB2 from binding to CSPG4^{mini}. In this two-step pull down assay, TcdB1 and TcdB2 were pre-bound to bezlotoxumab immobilized on protein A resins, which were then examined for binding to CSPG4^{mini}. FIG. 15C shows Bezlotoxumab can still bind to the CSPG4-bound TcdB1 and TcdB2. TcdB1 and TcdB2 were pre-bound to the biotin labeled CSPG4^{mini} immobilized on Strep-Tactin resins, which were then tested for bezlotoxumab binding. FIG. 15D shows TcdB2 could not bind CSPG4^{mini} when it was pre-bound to the immobilized bezlotoxumab according to ELI assays. FIG. 15E shows Bezlotoxumab could still bind TcdB2 when it was pre-bound to the immobilized CSPG4 Repeat1. Sequential loading of different proteins to the biosensor is indicated by different background shading.

FIGS. 16A, 16B, 16C, 16D, 16E, 16F and 16G shows bezlotoxumab competes with CSPG4 in an allosteric manner. FIG. 16A shows a structure model showing the binding of CSPG4 and bezlotoxumab (PDB: 4NP4) in TcdB holotoxin (PDB: 6OQ5). TcdB holotoxin and CSPG4 Repeat1 are shown as surface models with the GTD, CPD, DRBD, CROPs, and CSPG4 Repeat1. The two Fab fragments of bezlotoxumab are shown as cartoon models. E1 and E2 indicate the epitope-1 and epitope-2 for bezlotoxurnab in TcdB. A close-up view into the conflicting area between the Fab 1 bound at the E1 site and TcdB is shown in an oval box, while the Fab residues that sterically clash with TcdB. FIG. 16B shows a proposed model for allosteric interactions between CSPG4 and beziotoxurnab (Bezlo). FIG. 16C shows TcdB1 could not bind CSPG4^{mini} when it was pre-bound to the immobilized beziotoxurnab according to BLI assays. FIG. 16D shows Bezlotoxumab could still bind TcdB1 when it was pre-bound to the immobilized CSPG4 Repeat1. Sequential loading of different proteins to the biosensor is indicated by different background colors. FIG. 16E shows the protection effects of inhibitors against TcdB1 and TcdB2 were quantified by the cytopathic cell-rounding assay on HeLa cells. HeLa cells were incubated with TcdB1 (10 pM) or TcdB2 (100 pM) in the presence of serial-diluted bezlotoxumab (bezlo), its Fab (Fab), or Repeat1-Fc (Repeats), Percentage of rounded cells are plotted by inhibitor concentrations at 6 h. Error bars indicate mean±s.d. (n=3 biologically independent experiments). FIG. 16F and 16G show the protective effects of Repeat1-Fc and bezlotoxumab against TcdB1 and TcdB2 were examined in vivo using the cecum injection assay. TcdB1 (6 μg), TcdB2 (6 μg), TcdB1 or TcdB2 with Repeat1-Fc (30 μg) ar bezlotoxumab (52 μg), Repeat1-Fc alone (30 μg), or the PBS control was injected into the cecum of CD1 mice in vivo. The cecum tissues were harvested 6 h later, and the representative H&E staining (scale bar represents 100 μm) (FIG. 16F) and the histological scores (error bars indicate mean±SEM, PBS n=5, B1 n=13, B1+Repeat1 n=6, B1+Bezlo n=6, B2 n=15, 82+Repeat1 n=7, B2+Bezlo n=6, Repeat1 n=4) (FIG. 16G) are shown, P-values were calculated through post hoc analysis of a One-way ANOVA using Holm-Sidak's multiple comparison test: ****: p≤0.0001, ***: p≤0.001, **: p≤0.01, *: p≤0.05. Exact p values are presented in the source data.

FIGS. 17A, 17B, 17C, 17D, 17E, and 17F show the protection of bezlotoxumab, its Fab fragment, and Repeat1-Fc against TcdB1 and TcdB2. FIGS. 17A, 17B, and 17C shows the protection effects of bezlotoxumab, its Fab fragment, and Repeat1-Fc against TcdB1 and TcdB2 were tested by the cytopathic cell-rounding assay on HeLa cells. HeLa cells were incubated with TcdB1 (10 pM) in the presence of bezlotoxumab or its Fab (FIG. 17A); or with TcdB1 (10 pM) or TcdB2 (100 pM) in the presence of bezlotoxumab or Repeat1-Fc (FIGS. 17B and 17C). Percentages of rounded cells over time were recorded and plotted. Error bars indicate mean±s.d. (n=3). FIG. 17D and 17E show graphical representations of sequence conservation of key amino acids consisting of the epitope-1 (FIG. 17D; SEQ ID NO: 44) and epitope-2 (FIG. 17E; SEQ ID NO 45) of bezlotoxumab among 206 unique TcdB variants. The height of symbols at each position indicates the relative frequency of each amino acid at that position based on analyses of 206 unique TcdB variants. FIG. 17F shows the protective effects of Repeat1-Fc and bezlotoxurnab against TcdB1 and TcdB2 were examined in vivo using the cecum injection assay. TcdB1 (6 μg), TcdB2 (6 μg), TcdB1 or TcdB2 with Repeat1-Fc (30 μg) or bezlotoxumab (52 μg), Repeat1-Fc alone (30 μg), or the PBS control was injected into the cecum of CD1 mice in vivo. The cecum tissues were harvested 6 h later and subjected to histological analysis. Error bars indicate mean±s.d. (PBS n=5, B1 n=13, B1+Repeat1 n=6, B1+Bezlo n=6, B82 n=15, B2+Repeat1 n=7, B2+Bezlo n=6, Repeat1 n=4). p-values by One-way ANOVA: ****: p≤0.0001, ***: p≤0.001, **: p≤0.01, *: p≤0.05. The p-values of B1 vs. B1+Repeat 1, B1 vs, B1+Bezlo, B2 vs. B2+Repeat 1, B2 vs. B2+Bezlo for inflammatory cell infiltration are 0.0010, 0,0075, 0.2006, and 0.9979; for hemorrhagic congestion are 0.0707, <0.0001, 0.2771, and >0.9999; for epithelial disruption are <0.0001, <0.0001, 0.0562, and 0.9879; for submucosal edema are <0.0001, <0.0001, 0.0136, and 0.4560.

FIGS. 18A, 18B, 18C, and 18D show the sequence alignment between 12 major Tcd8 subtypes (see Table 7) highlighting the CSPG4-binding regions on the 0PD (FIG. 18A; SEQ ID NO: 32,SEQ ID NO: 36, SEQ ID NO; 33, SEQ ID NO: 40, SEQ ID NO: 43, SEQ ID NO; 34, SEQ ID NO: 35, SEQ ID NO: 38, SEQ ID NO: 37, SEQ ID NO: 39, SEQ ID NO: 41, and SEQ ID NO: 42, respectively, in order of appearance) and hinge (FIG. 18B; SEQ ID NO: 32, SEQ ID NO: 35, SEQ ID NO: 34, SEQ ID NO: 39, SEQ ID NO: 36, SEQ ID NO: 33, SEQ ID NO: 40, SEQ ID NO: 37, SEQ ID NO: 38, SEQ ID NO: 41, SEQ ID NO: 42, and SEQ ID NO: 43, respectively, in order of appearance). Residues involved in CSPG4 binding are labeled as triangles and stars for CPD and hinge region, respectively, FIGS. 18C and 18D show the VHH-50 binding residues are labeled as stars, FIG. 18C (SEQ ID NO: 32, SEQ ID NO: 36, SEQ ID NO: 34, SEQ ID NO: 43, SEQ ID NO: 33, SEQ ID NO: 40, SEQ ID NO: 41, SEQ ID NO: 42, SEQ ID NO: 35, SEQ ID NO: 39, SEQ ID NO: 37, and SEQ ID NO: 38, respectively, in order of appearance) and 18D (SEQ ID NO: 32, SEQ ID NO: 36, SEQ ID NO: 34, SEQ ID NO: 43, SEQ ID NO: 33, SEQ ID NO: 40, SEQ ID NO: 41, SEQ ID NO: 42, SEQ ID NO: 35, SEQ ID NO: 39, SEQ ID NO: 37, and SEQ ID NO: 38, respectively, in order of appearance) demonstrate that the 5D-binding epitope is highly conserved among known TcdB variants, so it can provide broad-spectrum protection. Note: The 12 sequences aligned herein are representative of each TcdB subfamilies (sequences of each to the TcdB subtypes are shown in Table 7).

FIGS. 19A, 19B, 19C, and 19D show Bio-layer interferometry (BLI) analyses of TcdB1 and TcdB2 binding to RDA1 or RDA1-h5D. Representative binding curves with RDA1 or RDA1-h5D (humanized VHH Table 8) as a ligand immobilized on anti-human IgG Fc capture (AHC) biosensors and TcdB1 or TcdB2 as the analytes. The concentrations of TcdB1 and TcdB2 examined were labeled in each panel. The shown binding analysis results are means ±s.d. from three independent experiments.

FIG. 20 shows a preliminary Cryo-EM structure of TedB2 in complex with a tri-specific inhibitor (RDA1-h5D) as described herein. It demonstrates that Repeat1 binds to TcdB2 in a way similar to that of TcdB1, and that BOTH Repeat1 and 5D can simultaneously bind TcdB2 exactly as designed. In this case, CRD and the Fc fragment were invisible, which may have very flexible conformations that can't be seen.This result also proves that BOTH Repeat1 and 5D can simultaneously bind TcdB1 in a similar manner.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder, or of one or more symptoms thereof. In certain cases, the beneficial effects that a subject derives from a prophylactic or therapeutic agent do not result in a cure of the disease or disorder.

As used herein, “clinical improvement” may refer to a noticeable reduction in the symptoms of a disorder, or cessation thereof.

A “therapeutically effective amount” refers to an amount that is sufficient to achieve the desired therapeutic result or to have an effect on undesired symptoms but is generally insufficient to cause intolerable adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

The compositions can be administered to a subject in a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically acceptable salt is used in the formulation to render the formulation isotonic. Examples of the pharmaceutically acceptable carrier include, but are not limited to, saline, Ringer's solution, and dextrose solution. The pH of the solution is preferably from about 5 to about 8, and more preferably from about 7 to about 7.5. Further carriers include sustained release preparations such as semi-permeable matrices of solid hydrophobic polymers containing the disclosed compounds, which matrices are in the form of shaped articles, e.g., films, liposornes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The pharmaceutical formulation can he administered in a number of ways depending on whether local or systemic treatment is desired, and on the area to be treated. A preferred mode of administration of the composition is parenterally, for example by intravenous drip, subcutaneous, intraperitoneal, or intramuscular injection. Other modes of administration may be topically (including rectally, intranasally), by inhalation or orally, or parenterally, for example by intravenous drip, subcutaneous, intraperitoneal, or intramuscular injection. The disclosed compounds can be administered orally, intravenously, intraperitoneally, intramuscularly, subcutaneously, intracavity, transdermally, sublingually or through buccal delivery.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

As used herein, “TcdB1” may refer to a toxin that is released from classic reference strain Clostridium difficile, VIP10463. In some embodiments, a TcdB1 subtype may be released from C. difficile strains that include but are not limited to strains such as the 630 strain. As used herein, “TcdB2” may refer to a toxin that is released from a hypervirulent Clostridium difficile strain UK1. In some embodiments, the TcdB2 subtype may be released from C. difficile strains that include but are not limited to strains such as the R20291 and the CD196.

As used herein “broad spectrum” may refer to the ability of a composition to neutralize most and/or all TcdB subtypes (including but not limited to subtypes listed in Table 7) from different C. difficile strains and new TcdB mutants that likely emerge in the future.

TABLE 7
Non-limiting examples of TcdB subtypes.

| TcdB | | SEQ |
|---|---|---|
| subtype_strains | Sequences | ID NO: |
| TcdB1_VPI10463 | MSLVNRKQLEKMANVRFRTQEDEYVAILDALEEYHNMSEN | 32 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTVVESAINDTLESFREN | |
| | LNDPRFDYNKFFRKRMEIIYDKQKNFINYYKAQREENPEL | |
| | IIDDIVKTYLSNEYSKEIDELNTYIEESLNKITQNSGNDV | |
| | RNFEEFKNGESFNLYEQELVERWNLAAASDILRISALKEI | |
| | GGMYLDVDMLPGIQPDLFESIEKPSSVTVDFWEMTKLEAI | |
| | MKYKEYIPEYTSEHFDMLDEEVQSSFESVLASKSDKSEIF | |
| | SSLGDMEASPLEVKIAFNSKGIINQGLISVKDSYCSNLIV | |
| | KQIENRYKILNNSLNPAISEDNDFNTTTNTFIDSIMAEAN | |
| | ADNGRFMMELGKYLRVGFFPDVKTTINLSGPEAYAAAYQD | |
| | LLMFKEGSMNIHLIEADLRNFEISKTNISQSTEQEMASLW | |
| | SFDDARAKAQFEEYKRNYFEGSLGEDDNLDFSQNIVVDKE | |
| | YLLEKISSLARSSERGYIHYIVQLQGDKISYEAACNLFAK | |
| | TPYDSVLFQKNIEDSEIAYYYNPGDGEIQEIDKYKIPSII | |
| | SDRPKIKLTFIGHGKDEFNTDIFAGFDVDSLSTEIEAAID | |
| | LAKEDISPKSIEINLLGCNMFSYSINVEETYPGKLLLKVK | |
| | DKISELMPSISQDSIIVSANQYEVRINSEGRRELLDHSGE | |
| | WINKEESIIKDISSKEYISFNPKENKITVKSKNLPELSTL | |
| | LQEIRNNSNSSDIELEEKVMLTECEINVISNIDTQIVEER | |
| | IEEAKNLTSDSINYIKDEFKLIESISDALCDLKQQNELED | |
| | SHFISFEDISETDEGFSIRFINKETGESIFVETEKTIFSE | |
| | YANHITEEISKIKGTIFDTVNGKLVKKVNLDTTHEVNTLN | |
| | AAFFIQSLIEYNSSKESLSNLSVAMKVQVYAQLFSTGLNT | |
| | ITDAAKVVELVSTALDETIDLLPTLSEGLPIIATIIDGVS | |
| | LGAAIKELSETSDPLLRQEIEAKIGIMAVNLTTATTAIIT | |
| | SSLGIASGFSILLVPLAGISAGIPSLVNNELVLRDKATKV | |
| | VDYFKHVSLVETEGVFTLLDDKIMMPQDDLVISEIDFNNN | |
| | SIVLGKCEIWRMEGGSGHTVTDDIDHFFSAPSITYREPHL | |
| | SIYDVLEVQKEELDLSKDLMVLPNAPNRVFAWETGWTPGL | |
| | RSLENDGTKLLDRIRDNYEGEFYWRYFAFIADALITTLKP | |
| | RYEDTNIRINLDSNTRSFIVPIITTEYIREKLSYSFYGSG | |
| | GTYALSLSQYNMGINIELSESDVWIIDVDNVVRDVTIESD | |
| | KIKKGDLIEGILSTLSIEENKIILNSHEINFSGEVNGSNG | |
| | FVSLTFSILEGINAIIEVDLLSKSYKLLISGELKILMLNS | |
| | NHIQQKIDYIGFNSELQKNIPYSFVDSEGKENGFINGSTK | |
| | EGLFVSELPDVVLISKVYMDDSKPSFGYYSNNLKDVKVIT | |
| | KDNVNILTGYYLKDDIKISLSLTLQDEKTIKLNSVHLDES | |
| | GVAEILKFMNRKGNTNTSDSLMSFLESMNIKSIFVNFLQS | |
| | NIKFILDANFIISGTTSIGQFEFICDENDNIQPYFIKFNT | |
| | LETNYTLYVGNRQNMIVEPNYDLDDSGDISSTVINFSQKY | |
| | LYGIDSCVNKVVISPNIYTDEINITPVYETNNTYPEVIVL | |
| | DANYINEKINVNINDLSIRYVWSNDGNDFILMSTSEENKV | |
| | SQVKIRFVNVFKDKTLANKLSFNFSDKQDVPVSEIILSFT | |
| | PSYYEDGLIGYDLGLVSLYNEKFYINNFGMMVSGLIYIND | |
| | SLYYFKPPVNNLITGFVTVGDDKYYFNPINGGAASIGETI | |
| | IDDKNYYFNQSGVLQTGVFSTEDGFKYFAPANTLDENLEG | |
| | EAIDFTGKLIIDENIYYFDDNYRGAVEWKELDGEMHYFSP | |
| | ETGKAFKGLNQIGDYKYYFNSDGVMQKGFVSINDNKHYFD | |
| | DSGVMKVGYTEIDGKHFYFAENGEMQIGVENTEDGFKYFA | |
| | HHNEDLGNEEGEEISYSGILNFNNKIYYFDDSFTAVVGWK | |
| | DLEDGSKYYFDEDTAEAYIGLSLINDGQYYFNDDGIMQVG | |
| | FVTINDKVFYFSDSGIIESGVQNIDDNYFYIDDNGIVQIG | |
| | VFDTSDGYKYFAPANTVNDNIYGQAVEYSGLVRVGEDVYY | |
| | FGETYTIETGWIYDMENESDKYYFNPETKKACKGINLIDD | |
| | IKYYFDEKGIMRTGLISFENNNYYFNENGEMQFGYINIED | |
| | KMFYFGEDGVMQIGVENTPDGFKYFAHQNTLDENFEGESI | |
| | NYTGWLDLDEKRYYFTDEYIAATGSVIIDGEEYYFDPDTA | |
| | QLVISE | |
| TcdB2_R20291 | MSLVNRKQLEKMANVRFRVQEDEYVAILDALEEYHNMSEN | 33 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTIVESATNDTLESFREN | |
| | LNDPRFDYNKFYRKRMEIIYDKQKNFINYYKTQREENPDL | |
| | IIDDIVKIYLSNEYSKDIDELNSYIEESLNKVTENSGNDV | |
| | RNFEEFKGGESFKLYEQELVERWNLAAASDILRISALKEV | |
| | GGVYLDVDMLPGIQPDLFESIEKPSSVTVDFWEMVKLEAI | |
| | MKYKEYIPGYTSEHFDMLDEEVQSSFESVLASKSDKSEIF | |
| | SSLGDMEASPLEVKIAFNSKGIINQGLISVKDSYCSNLIV | |
| | KQIENRYKILNNSLNPAISEDNDFNTTTNAFIDSIMAEAN | |
| | ADNGRFMMELGKYLRVGFFPDVKTTINLSGPEAYAAAYQD | |
| | LLMFKEGSMNIHLIEADLRNFEISKTNISQSTEQEMASLW | |
| | SFDDARAKAQFEEYKKNYFEGSLGEDDNLDFSQNTVVDKE | |
| | YLLEKISSLARSSERGYIHYIVQLQGDKISYEAACNLFAK | |
| | TPYDSVLFQKNIEDSEIAYYYNPGDGEIQEIDKYKIPSII | |
| | SDRPKIKLTFIGHGKDEFNTDIFAGLDVDSLSTEIETAID | |
| | LAKEDISPKSIEINLLGCNMFSYSVNVEETYPGKLLLRVK | |
| | DKVSELMPSISQDSIIVSANQYEVRINSEGRRELLDHSGE | |
| | WINKEESIIKDISSKEYISFNPKENKIIVKSKNLPELSTL | |
| | LQEIRNNSNSSDIELEEKVMLAECEINVISNIDTQVVEGR | |
| | IEEAKSLTSDSINYIKNEFKLIESISDALYDLKQQNELEE | |
| | SHFISFEDILETDEGFSIRFIDKETGESIFVETEKAIFSE | |
| | YANHITEEISKIKGTIFDTVNGKLVKKVNLDATHEVNTLN | |
| | AAFFIQSLIEYNSSKESLSNLSVAMKVQVYAQLFSTGLNT | |
| | ITDAAKVVELVSTALDETIDLLPTLSEGLPVIATIIDGVS | |
| | LGAAIKELSETSDPLLRQEIEAKIGIMAVNLTAATTAIIT | |
| | SSLGIASGFSILLVPLAGISAGIPSLVNNELILRDKATKV | |
| | VDYFSHISLAESEGAFTSLDDKIMMPQDDLVISEIDFNNN | |
| | SITLGKCEIWRMEGGSGHTVTDDIDHFFSAPSITYREPHL | |
| | SIYDVLEVQKEELDLSKDLMVLPNAPNRVFAWETGWTPGL | |
| | RSLENDGTKLLDRIRDNYEGEFYWRYFAFIADALITTLKP | |
| | RYEDTNIRINLDSNTRSFIVPVITTEYIREKLSYSFYGSG | |
| | GTYALSLSQYNMNINIELNENDTWVIDVDNVVRDVTIESD | |
| | KIKKGDLIENILSKLSIEDNKILDNHEINFSGTLNGGNGF | |
| | VSLTFSILEGINAVIEVDLLSKSYKVLISGELKTLMANSN | |
| | SVQQKIDYIGLNSELQKNIPYSFMDDKGKENGFINCSTKE | |
| | GLFVSELSDVVLISKVYMDNSKPLFGYCSNDLKDVKVITK | |
| | DDVIILTGYYLKDDIKISLSFTIQDENTIKLNGVYLDENG | |
| | VAEILKFMNKKGSTNTSDSLMSFLESMNIKSIFINSLQSN | |
| | TKLILDTNFIISGTTSIGQFEFICDKDNNIQPYFIKFNTL | |
| | ETKYTLYVGNRQNMIVEPNYDLDDSGDISSTVINFSQKYL | |
| | YGIDSCVNKVIISPNIYTDEINITPIYEANNTYPEVIVLD | |
| | TNYISEKINININDLSIRYVWSNDGSDFILMSTDEENKVS | |
| | QVKIRFTNVFKGNTISDKISFNFSDKQDVSINKVISTFTP | |
| | SYYVEGLLNYDLGLISLYNEKFYINNFGMMVSGLVYINDS | |
| | LYYFKPPIKNLITGFTTIGDDKYYFNPDNGGAASVGETII | |
| | DGKNYYFSQNGVLQTGVFSTEDGFKYFAPADTLDENLEGE | |
| | AIDFTGKLTIDENVYYFGDNYRAAIEWQTLDDEVYYFSTD | |
| | TGRAFKGLNQIGDDKFYFNSDGIMQKGFVNINDKTFYFDD | |
| | SGVMKSGYTEIDGKYFYFAENGEMQIGVFNTADGFKYFAH | |
| | HDEDLGNEEGEALSYSGILNFNNKIYYFDDSFTAVVGWKD | |
| | LEDGSKYYFDEDTAEAYIGISIINDGKYYFNDSGIMQIGF | |
| | VTINNEVFYFSDSGIVESGMQNIDDNYFYIDENGLVQIGV | |
| | FDTSDGYKYFAPANTVNDNIYGQAVEYSGLVRVGEDVYYF | |
| | GETYTIETGWIYDMENESDKYYFDPETKKAYKGINVIDDI | |
| | KYYFDENGIMRTGLITFEDNHYYFNEDGIMQYGYLNIEDK | |
| | TFYFSEDGIMQIGVFNTPDGFKYFAHQNTLDENFEGESIN | |
| | YTGWLDLDEKRYYFTDEYIAATGSVIIDGEEYYFDPDTAQ | |
| | LVISE | |
| TcdB3_M120 | MSLVNRKQLEKMANVRFRTQEDEYVAILDALEEYHNMSEN | 34 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTIVESAINDTLESFREN | |
| | LNNPRFDYNKFFRKRMEIIYDKQKNFINYYKAQREENPEL | |
| | IIDDIVKIYLSNEYSKEIDELNTYIEESLNKIKQNSGNDV | |
| | RNFEEFKNGESFKLYEQELVERWNLAAASDILRISALKEI | |
| | GGMYLDVDMLPGIQPDLFESIEKPSSVTVDFWEMTKLEAI | |
| | MKYKEYIPGYTSEHFDMLDEEVQSSFESALASKSDKSEIF | |
| | SSLGDMEASPLEVKIAFNSKGIINQGLISVKDSYCSNLIV | |
| | KQIENRYKILNNSLNPAISEDNDFNTTTNTFIDSIMAEAN | |
| | ADNGRFMMELGKYLRVGFFPDVKTTVNLSGPEAYAAAYQD | |
| | LLMFKEGSMNIHLIEADLRNFEISKTNISQSTEQEMASLW | |
| | TFDDARAKVQFEEYKRNYFEGSLGEDDNLDFSQNIVVDKE | |
| | YLLEKISSLARSSERGYIHYIVQLQGDKISYEAACNLFAK | |
| | TPYDSILFQKNIENSEVAYYYNPGDGEIQEIDKYRIPSII | |
| | SDRPKIKLTFIGHGKDEFNTDIFAGLDVDSLSTEIETAID | |
| | LAKEDISSKSIEINLLGQNMFSYSINVEETYPGKLLLKVK | |
| | DKISELMPSISQDSIIVSANQYEVRINNEGRRELLDHSGE | |
| | WINKEESIIKDISSKEYISFNPKENKITVKSKNLPELSTL | |
| | LQEIRNNSNLSDIELEEKVMLAECEINVISNIDTQIVEER | |
| | IEEAKNLTSDSINYIKNEFKLIESISDSLYDLKQQNELDD | |
| | SHFISFEDISKTEDGFSIRFINKETGESIFVETEKEIFSE | |
| | YANHIEREISNIKDTIFDTVNGKLVKKVNLDAIHEVNTLN | |
| | AAFFIQSLIGYSSSKESLSNLSVAMKVQVYAQLFSTGLNT | |
| | ITDAAKVVELVSTALDETIDLLPTLSEGLPVIATIIDGVS | |
| | LGAAIKELSETSDPLLRQEIEAKIGIMAVNLTAATTAIIT | |
| | SSLGIASGFSILLVPLAGISAGIPSLVNNELVLRDKATKV | |
| | VDYFKHISLVETEGAFTLLDDKIMIPQDDLVISEIDFNNN | |
| | SIVLGKCEIWRMEGGSGHTVTNDIDHFFSSPTITYIKPHL | |
| | SIYDVLEVQKEELDLSKDLMVLPNAPNRVFAWETGWTPGL | |
| | RSLENEGTKLLDRIRDHYKGEFYWRYFAFIADALITTLKP | |
| | RYEDTNIRINLDSNNRSFIVPIITTEHIREKLSYSFHGSG | |
| | GTYALSLSQYNMGINIELSESDVWIIDVDNVVRDVTIDSD | |
| | KIKKGDLIEGILSTLSIEDNKIILNHHEINFSGDVNGSNG | |
| | FISLTFSILEGINAIIEVDLLSKSYKLLISGELKILMLNS | |
| | NHIQQKIDYIGFNSELQKNIPYSFVDSEGKENGFINGSTK | |
| | EGLFVSELPDWVLISKVYMDDSKPSFGYYSNNLKDVKVIT | |
| | KDNVNILTGYYLKDDIKISLSFTLQDEKTIKLNGVHLDES | |
| | GVAEILKFMNKKGSTNTSDSLMSFLESVNIKSIFVNFLQS | |
| | KINFILDANFIISGTTSIGQFEFICDENDNIQPYFIKFNT | |
| | LETTYTLYVGNRQNMIVEPNYDLDDSGDISSTVINFSQKY | |
| | LYGIDSCVNKVVISPNIYTDEINITPVYETNNNYPEVIVL | |
| | DANYINEKINVNINDLSIRYVWSNDGNDFILMSTSEENKV | |
| | SQVKIRFVNVFKDKTLANKLSFNFSDKQDVPVSEIISAFT | |
| | PSYYEDGLIGYDLGLVSLYNEKFYINNFGMMVSGLIYIND | |
| | SLYYFKPPVNNLITGFVTVGDDKYYFNPTNGGAASIGETI | |
| | INDKNYYFNQSGILQTGVFSTEDGLKYFAPANTLDENLEG | |
| | EAIDFTGKLIIDENIYYFEDNYRGAVEWKELDGEMYYFSP | |
| | ETGKAFKGLNQIGDDKYYFNSDGIMQKGFVSINDKKYYFD | |
| | DSGVMKVGYIEIDGKYFYFAENGEMQIGVENTSDGFKYFA | |
| | HHNEDLGNEEGEAISYSGILNFNNKIYYFDYSFTAVVGWK | |
| | DLEDGSKYYFDEDTAEAYVGLSLINDGQYYFNDDGIMQVG | |
| | FVTINNKVFYFSDSGIIESGVQNIDDNYFYIDEKGIVQIG | |
| | VFDTSDEYKYFAPANTVNDNIYGQAVDYSGLVRVGEDIYY | |
| | FGETYTIETGWIYDMENESDKYYFNPETKKACKGINLIDD | |
| | IKYYFDENGIMRTGLISFENNDYYFNENGEMQFGYINIED | |
| | KMFYFGEDGVMQIGVFNTQDGFKYFAHQNTLDENFEGESI | |
| | NYTGWLDLDEKRYYFTDEYIAATGSVIIDGEEYYFDPDTA | |
| | QLVISE | |
| TcdB4_1470 | MSLVNRKQLEKMANVRFRVQEDEYVAILDALEEYHNMSEN | 35 |
| | TVVEKYLKLKDINSLTDTYIDTYKKSGRNKALKKFKEYLV | |
| | IEILELKNSNLTPVEKNLHFIWIGGQINDTAINYINQWKD | |
| | VNSDYNVNVFYDSNAFLINTLKKTIIESASNDTLESFREN | |
| | LNDPEFNHTAFFRKRMQIIYDKQQNFINYYKAQKEENPDL | |
| | IIDDIVKTYLSNEYSKDIDELNAYIEESLNKVTENSGNDV | |
| | RNFEEFKTGEVFNLYEQELVERWNLAGASDILRVAILKNI | |
| | GGVYLDVDMLPGIHPDLFKDINKPDSVKTAVDWEEMQLEA | |
| | IMKHKEYIPEYTSKHFDTLDEEVQSSFESVLASKSDKSEI | |
| | FLPLGDIEVSPLEVKIAFAKGSIINQALISAKDSYCSDLL | |
| | IKQIQNRYKILNDTLGPIISQGNDFNTTMNNFGESLGAIA | |
| | NEENISFIAKIGSYLRVGFYPEANTTITLSGPTIYAGAYK | |
| | DLLTFKEMSIDTSILSSELRNFEFPKVNISQATEQEKNSL | |
| | WQFNEERAKIQFEEYKKNYFEGALGEDDNLDFSQNTVTDK | |
| | EYLLEKISSSTKSSERGYVHYIVQLQGDKISYEAACNLFA | |
| | KNPYDSILFQKNIEDSEVAYYYNPTDSEIQEIDKYRIPDR | |
| | ISDRPKIKLTFIGHGKAEFNTDIFAGLDVDSLSSEIETAI | |
| | GLAKEDISPKSIEINLLGQNMFSYSVNVEETYPGKLLLRV | |
| | KDKVSELMPSMSQDSIIVSANQYEVRINSEGRRELLDHSG | |
| | EWINKEESIIKDISSKEYISFNPKENKIIVKSKNLPELST | |
| | LLQEIRNNSNSSDIELEEKVMLAECEINVISNIETQVVEE | |
| | RIEEAKSLTSDSINYIKNEFKLIESISDALCDLKQQNELE | |
| | DSHFISFEDISETDEGFSIRFINKETGESIFVETEKTIFS | |
| | EYANHITEEISKIKGTIFDTVNGKLVKKVNLDTTHEVNTL | |
| | NAAFFIQSLIEYNSSKESLSNLSVAMKVQVYAQLFSTGLN | |
| | TITDAAKVVELVSTALDETIDLLPTLSEGLPIIATIIDGV | |
| | SLGAAIKELSETSDPLLRQEIEAKIGIMAVNLTTATTAII | |
| | TSSLGIASGFSILLVPLAGISAGIPSLVNNELVLRDKATK | |
| | VVDYFKHVSLVETEGVFTLLDDKVMMPQDDLVISEIDFNN | |
| | NSIVLGKCEIWRMEGGSGHTVTDDIDHFFSÅPSITYREPH | |
| | LSIYDVLEVQKEELDLSKDLMVLPNAPNRVFAWETGWTPG | |
| | LRSLENDGTKLLDRIRDNYEGEFYWRYFAFIADALITTLK | |
| | PRYEDTNIRINLDSNTRSFIVPIITTEYIREKLSYSFYGS | |
| | GGTYALSLSQYNMGINIELSESDVWIIDVDNVVRDVTIES | |
| | DKIKKGDLIEGILSTLSIEENKIILNSHEINFSGEVNGSN | |
| | GFVSLTFSILEGINAIIEVDLLSKSYKLLISGELKILMLN | |
| | SNHIQQKIDYIGFNSELQKNIPYSFVDSEGKENGFINGST | |
| | KEGLFVSELPDVVLISKVYMDDSKPSFGYYSNNLKDVKVI | |
| | TKDNVNILTGYYLKDDIKISLSLTLQDEKTIKLNSVHLDE | |
| | SGVAEILKFMNRKGSTNTSDSLMSFLESMNIKSIFVNFLQ | |
| | SNIKFILDANFIISGTTSIGQFEFICDENNNIQPYFIKFN | |
| | TLETNYTLYVGNRQNMIVEPNYDLDDSGDISSTVINFSQK | |
| | YLYGIDSCVNKVVISPNIYTDEINITPVYETNNTYPEVIV | |
| | LDANYINEKINVNINDLSIRYVWSNDGNDFILMSTSEENK | |
| | VSQVKIRFVNVFKDKTLANKLSFNFSDKQDVPVSEIILSF | |
| | TPSYYEDGLIGYDLGLVSLYNEKFYINNFGMMVSGLIYIN | |
| | DSLYYFKPPVNNLITGFVTVGDDKYYFNPINGGAASIGET | |
| | IIDDKNYYFNQSGVLQTGVFSTEDGFKYFAPANTLDENLE | |
| | GEAIDFTGKLIIDENIYYFEDNYRGAVEWKELDGEMHYFS | |
| | PETGKAFKGLNQIGDDKYYFNSDGVMQKGFVSINDNKHYF | |
| | DDSGVMKVGYTEIDGKHFYFAENGEMQIGVENTEDGFKYF | |
| | AHHNEDLGNEEGEEISYSGILNFNNKIYYFDDSFTAVVGW | |
| | KDLEDGSKYYFDEDTAEAYIGLSLINDGQYYFNDDGIMQV | |
| | GFVTINDKVFYFSDSGIIESGVQNIDDNYFYIDDNGIVQI | |
| | GVFDTSDGYKYFAPANTVNDNIYGQAVEYSGLVRVGEDVY | |
| | YFGETYTIETGWIYDMENESDKYYFDPETKKACKGINLID | |
| | DIKYYFDEKGIMRTGLISFENNNYYFNENGEMQFGYINIE | |
| | DKMFYFGEDGVMQIGVFNTPDGFKYFAHQNTLDENFEGES | |
| | INYTGWLDLDEKRYYFTDEYIAATGSVIIDGEEYYFDPDT | |
| | AQLVISE | |
| TcdB5_55767 | MSLVNRKQLEKMANVRFRTQEDEYVAILDALEEYHNMSEN | 36 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTIVESATNDTLESFREN | |
| | LNDPRFDYNKFFRKRMEIIYDKQKNFINYYKAQREENPEL | |
| | IIDDIVKTYLSNEYSKEIDELNAYIEESLNKITQNSGNDV | |
| | RNFEEFKNGESFNLYEQELVERWNLAAASDILRISALKEI | |
| | GGVYLDVDMLPGIQPDLFESIEKPSSVTVDFWEMTKLEAI | |
| | MKYKEYIPGYTSEHFDMLDEEVQSSFESALASKSDKSEIF | |
| | SSLGDMEASPLEVKIAFNSKGIINQGLISVKDSYCSNLIV | |
| | KQIENRYKILNNSLNPAISEDNDFNTTTNAFIDSIMAEAN | |
| | ADNGRFMMELGKYLRVGFFPDVKTTINLSGPEAYAAAYQD | |
| | LLMFKEDSMNIHLIEADLRNFEIPKTNISQSTEQEMASLW | |
| | SFDDARAKAQFEEYKRNYFEGSLGEDDNLDFSQNIVVDKE | |
| | YLLEKISSLARSSERGYIHYIVQLQGDKISYEAACNLFAK | |
| | TPYDSILFQKNIEDSEIAYYYNPGDGEIQEIDKYKIPSII | |
| | SDRPKIKLTFIGHGKDEFNTDIFAGLDVDSLSTEIETAID | |
| | LAKEDISPKSIEINLLGCNMFSYSINVEETYPGKLLLKVK | |
| | DKISELMPSISQDSIIVSANQYEVRINSEGRRELLDHSGE | |
| | WINKEESIIKDISSKEYISFNPKENKITVKSKNLPELSTL | |
| | LQEIRNNSNSSDIELEEKVMLTECEINVISNIDTQIVEER | |
| | IEEAKNLTSDSINYIKNEFKLIESISDALCDLKQQNELDD | |
| | SHFISFEDISETDEGFSIRFINKETGESIFVETEKTIFSE | |
| | YANHITEEISKIKDTIFDTVNGKLVKKVNLDTTHEVNTLN | |
| | AAFFIQSLIEYNSSKESLSNLSVAMKVQVYAQLFSTGLNT | |
| | ITDAAKVVELVSTALDETIDLLPTLSEGLPVIATIIDGVS | |
| | LGAAIKELSETSDPLLRQEIEAKIGIMAVNLTAATTAIIT | |
| | SSLGIASGFSILLVPLAGISAGIPSLVNNELVLRDKATKV | |
| | VDYFKHVSLVETEGVFTLLDDKIMMPQDDLVISEIDFNNN | |
| | SIVLGKCEIWRMEGGSGHTITDDIDHFFSAPSITYREPHL | |
| | SIYDVLEVQKEELDLSKDLMVLPNAPNRVFAWETGWTPGL | |
| | RSLENDGTKLLDRIRDHYEGEFYWRYFAFIADALITTLKP | |
| | RYEDTNIRINLQSNTRSFIVPIITTEYIREKLSYSFYGSG | |
| | GTYALSLSQYNMGINIELSESDVWIIDVDNVVRDVTIESD | |
| | KIKKGDLIEGILSTLSIEENKIILNSHEINFSGDVNGSNG | |
| | FVSLTFSILEGINAIIEVDLLSKSYKLLISGELKILMLNS | |
| | NHIQQKIDYIGFNSELQKNIPYSFVNDEGKENGFINGSTK | |
| | EGLFVSELPDVVLISKVYMDDSKPSFGYYSNNLKDVKVIT | |
| | KDDVNILTGYYLKDDIKISLSLTLQDEKTIKLNSVHLDES | |
| | GVAEILKFMNKKGSTNTSDSLMSFLESMNIKSIFVNFLQS | |
| | NIKFILDTNFIISGTTSIGQFEFICDENDNIQPYFIKFNT | |
| | LETNYTLYVGNRQNMIVEPNYDLDDSGDISSTVINFSQKY | |
| | LYGIDSCVNKVVISPNIYTDEINITPVYETNNTYPEVIVL | |
| | DANYINEKINVNINDLSIRYVWSNDGNDFILMSTSEENKV | |
| | SQVKIRFVNVFKDKTLANKLSFNFSDKQDVPVSKIISSFT | |
| | PSYYDGGLIGYGLGLVSLYNEKFYINNFGMMVSGLIYIND | |
| | SLYYFKPPVNNLITGFMTVGDDKYYFNPTNGGAASIGETI | |
| | IDDKNYYFNQSGVLQTGVFSTEDGFKYFAPANTLDENLEG | |
| | EAIDFTGKLIIDENIYYFEDNYRGAVEWKELDGEMYYFSP | |
| | ETGKAFKGLNQIGDDKYYFNSDGVMQKGFVSINDKKHYFD | |
| | DSGVMKVGYTEIDGKYFYFAENGEMQIGVENTEDGFKYFA | |
| | HHNEDLGNEEGEEISYSGILNFNNKIYYFDDSFTAVVGWK | |
| | DLEDGSKYYFDEDTAEAYIGLSLINDGQYYFNDDGIMQVG | |
| | FVAINDKVFYFSDSGIIESGVQNIDDNYFYIDEKGIVQIG | |
| | VFDTSDGYKYFAPANTVNDNIYGQAVEYSGLVRVGEDVYY | |
| | FGETYTIETGWIYDMENESDKYYFNPETKKACKGINLIDD | |
| | IKYYFDENGIMRTGLISFENNDYYFNENGEMQFGYINIED | |
| | KMFYFGEDGVMQIGVFNTPDGFKYFAHQNTLDENFEGESI | |
| | NYTGWLDLDEKRYYFTDEYIAATGSVIIDGEEYYFDPDTA | |
| | QLVISE | |
| TcdB6_51680 | MSLVNRKQLEKMANVRFRVQEDEYVAILDALEEYHNMSEN | 37 |
| | TVVEKYLKLKDINSLTDTYIDTYKKSGRNKALKKFKEYLV | |
| | TEILELKNSNLTPVEKNLHFIWIGGQINDTAINYINQWKD | |
| | VNSDYNVNVFYDSNAFLINTLKKTIIESASNDTLESFREN | |
| | LNDPEFNHTAFFRKRMQIIYDKQQNFINYYKAQKEENPDL | |
| | IIDDIVKTYLSNEYSKDIDELNAYIEESLNKVTENSGNDV | |
| | RNFEEFKTGEVFNLYEQELVERWNLAGASDILRVAILKNI | |
| | GGVYLDVDMLPGIHPDLFKDINKPDSVKTAVDWEEMQLEA | |
| | IMKYKEYIPEYTSKHFDTLDEEVQSSFESVLASKSDKSEI | |
| | FLPLGDIEVSPLEVKIAFAKGSIINQALISAKDSYCSDLL | |
| | IKQIQNRYKILNDTLGPIISQGNDFNTTMNNFGESLGAIA | |
| | NEENISFIAKIGSYLRVGFYPEANTTITLSGPTIYAGAYK | |
| | DLLTFKEMSIDTSILSSELRNFEFPKVNISQATEQEKNSL | |
| | WQFNEERAKIQFEEYKKNYFEGALGEDDNLDFSQNTVIDK | |
| | EYLLEKISSSTKSSERGYVHYIVQLQGDKISYEAACNLFA | |
| | KNPYDSILFQKNIEDSEVAYYYNPTDSEIQEIDKYRIPDR | |
| | ISDRPKIKLTLIGHGKAEFNTDIFAGLDVDSLSSEIETII | |
| | DLAKADISPKSIEINLLGCNMFSYSVNVEETYPGKLLLRV | |
| | KDKVSELMPSISQDSIIVSANQYEVRINSEGRRELLDHSG | |
| | EWINKEESIIKDISSKEYISFNPKENKIIVKSKNLPELST | |
| | LLQEIRNNSNSSDIELEEKVMLAECEINVISNIETQVVEE | |
| | RIEEAKSLTSDSINYIKNEFKLIESISDALYDLKQQNELE | |
| | ESHFISFEDISETDEGFSIRFIDKETGESIFVETEKAIFS | |
| | EYANHITEEISKIKGTIFDTVNGKLVKKVNLDATHEVNTL | |
| | NAAFFIQSLIEYNSSKESLSNLSVAMKVQVYAQLFSTGLN | |
| | TITDAAKVVELVSTALDETIDLLPTLSEGLPVIATIIDGV | |
| | SLGAAIKELSETSDPLLRQEIEAKIGIMAVNLTAATTAII | |
| | TSSLGIASGFSILLVPLAGISAGIPSLVNNELILRDKATK | |
| | VVDYFSHISLAESEGAFTSLDDKIMMPQDDLVISEIDFNN | |
| | NSITLGKCEIWRMEGGSGHTVTDDIDHFFSSPSITYREPH | |
| | LSIYDVLEVKKEELDLSKDLMVLPNAPNRVFGWETGWTPG | |
| | LRGLENDGTKLLDRIRDQYEGQFYWRFFAFIADALITTLK | |
| | PRYEDTNVRISLDSNTRSFIVPVITTEYIREKLSYSFYGS | |
| | GGTYALSLSQYNMNINIELNENDTWVIDVDNVVRSVTIES | |
| | DKIKKGDLIENILSKLSIEDNKIILDNHEINFSGTLNGGN | |
| | GFVSLTFSILEGINAVIEVDLLSKSYKVLISGELKTLMAN | |
| | SNSVQQKIDYIGLNSELQKNIPYSFMDDKGKENGFINCST | |
| | KEGLFVSELSDVVLIIKVYMDNSKPPFGYYSNDLKDVKAI | |
| | TKDDVIILTGYYLKDDIKISLSFTIQDKNTIKLNGVYLDE | |
| | NGVAEILKFMNKKGSTNTSDSLMSFLESMNIKSIFIKSLK | |
| | SNAKLILDTNFIISGTTSIGQFEFICDKDNNIQPYFIKFN | |
| | TLETKYTLYVGNRQNMIVEPNYDLQDSGDISSTVINFSQK | |
| | YLYGIDSCVNKVIISPNIYTDEINITPIYEANNTYPEVIV | |
| | LDTNYISEKINININDLSIRYVWSNDGSDFILMSTDEENK | |
| | VSQVKIRFTNVFKGNTISDKISFNFSDKQDVSINKIISTF | |
| | TPSYYVEGLLNYHLGLISLYNEKFYINNFGMMVSGLVYIN | |
| | DSLYYFKPPIKNLITGFTTIGDDKYYFNPDNGGAASVGET | |
| | IIDGKNYYFSPNGVLQTGVFSTEDGFKYFAPADTLDENLE | |
| | GEAIDFTGKLIIDENVYYFGDNYRAAIEWQTLDDEVYYFS | |
| | TDTGRAFKGLNQIGDDKFYFNSDGIMQKGFVNINDKTFYF | |
| | DDSGVMKSGYTEIDGKHFYFAENGEMQIGVFNTADGFKYF | |
| | AHHDEDLGNEEGEALSYSGILNFNNKIYYFDDSFTAVVGW | |
| | KDSEDGSKYYFDEDTAEAYIGISTINDGKYYFNDSGIMQI | |
| | GFVTINNEVFYFSDSGIVESGMQNIDDNYFYIDENGLVQI | |
| | GVFDTSDGYKYFAPANTVNDNIYGQAVEYSGLVRVGEDVY | |
| | YFGETYTIETGWIYDMENESDKYYFDPETKKAYKGINVID | |
| | DIKYYFDENGIMRTGLITFEDNHYYFNEDGIMQYGYINIE | |
| | DKMFYFNEDGVMQIGVENTADGFKYFAHQNTLDENFEGES | |
| | INYTGWLDLDEKRYYFTDEYIAAIGSVIIDGEEYYFDPDT | |
| | AELVISE | |
| TcdB7_8864 | MSLVNRKQLEKMANVRFRVQEDEYVAILDALEEYHNMSEN | 38 |
| | TVVEKYLKLKDINSLTDTYIDTYKKSGRNKALKKFKEYLV | |
| | TEILELKNSNLTPVEKNLHFIWIGGQINDTAINYINQWKD | |
| | VNSDYNVNVFYDSNAFLINTLKKTIIESASNDTLESFREN | |
| | LNDPEFNHTAFFRKRMQIIYDKQQNFINYYKAQKEENPDL | |
| | IIDDIVKTYLSNEYSKDIDELNAYIEESLNKVTENSGNDV | |
| | RNFEEFKTGEVFNLYEQELVERWNLAGASDILRVAILKNI | |
| | GGVYLDVDMLPGIHPDLFKDINKPDSVKTAVDWEEMQLEA | |
| | IMKYKEYIPEYTSKHFDTLDEEVQSSFESVLASKSDKSEI | |
| | FLPLGDIEVSPLEVKVAFAKGSIINQALISAKDSYCSDLL | |
| | IKQIQNRYKILNDTLGPIISQGNDFNTTMNNFGESLGAIA | |
| | NEENISFIAKIGSYLRVGFYPEANTTITLSGPTIYAGAYK | |
| | DLLTFKEMSIDTSILSSELRNFEFPKVNISQATEQEKNSL | |
| | WQFNEERAKIQFEEYKKNYFEGALGEDDNLDFSQNTVTDK | |
| | EYLLEKISSSTKSSERGYVHYIVQLQGDKISYEAACNLFA | |
| | KNPYDSILFQKNIEDSEVAYYYNPTDSEIQEIDKYRIPDR | |
| | ISDRPKIKLTLIGHGKAEFNTDIFAGLDVDSLSSEIETII | |
| | DLAKADISPKSIEINLLGQNMFSYSVNVEETYPGKLLLRV | |
| | KDKVSELMPSISQDSIIVSANQYEVRINSEGRRELLDHSG | |
| | EWINKEESIIKDISSKEYISFNPKENKIIVKSKNLPELST | |
| | LLQEIRNNSNSSDIELEEKVMLAECEINVISNIETQVVEE | |
| | RIEEAKSLTSDSINYIKNEFKLIESISDALYDLKQQNELE | |
| | ESHFISFEDISKTDEGFSIRFIDKETGESIFVETEKAIFS | |
| | EYANHITEEISKLKDTIFDTVNGKLVKKVTLDATHEVNTL | |
| | NAAFFIQSLIGYNSSKESLSNLSVAMKVQVYAQLFSTGLN | |
| | TITDAAKVVELVSTALDETIDLLPTLSEGLPVIATIIDGV | |
| | SLGASIKELSETSDPLLRQEIEAKIGIMAVNLTAATTAII | |
| | TSSLGIASGFSILLVPLAGISAGIPSLVNNELILRAEAKN | |
| | VVDYFGHISLAESEGAFTLLDDKIMMPQDDLVISEIDFNN | |
| | NSITLGKCEIWRMEGGSGHTVTDDIDHFFSAPSTTYREPY | |
| | LSIYDVLDVKKEELDLSKDLMVLPNAPDRIFGWERGWTPG | |
| | LRSLENDGTKLLDRIRDHYEGQFYWRFFAFIADSVITKLK | |
| | PRYEDTNIRISLDSNTRSFIVPVITTEYIREKLSYSFYGS | |
| | GGTYALSLSQYNMNINIELNENDTWVIDVDNVVRDVTIES | |
| | DKIKKGDLIENILSKLSIEDNKIILDNHEINFSGTLNGGN | |
| | GFVSLTFSILEGINAVIEVDLLSKSYKVLISGELKTLMAN | |
| | SNSVQQKIDYIGLNSELQKNIPYSFMDDEGKENGFINCFT | |
| | KEGLFVSELSDVVLIIKVYMDNSKPPFGYYSNDLKDVKVI | |
| | TKDDVIILTGYYLKDDIKISLSFTIQDKNTIKLNGVYLDE | |
| | NGVAEILKFMNKKGSTNTSDSLMSFLESMNIKSIFIKSLK | |
| | SNAKLILDTNFIISGTTSIGQFEFICDKDNNIQPYFIKFN | |
| | TLETKYTLYVGNRQNMIVEPNYNLDDSGDISSTVINFSQK | |
| | YLYGIDSCVNKVIISPNIYTDEINITPVHEANNTYPEVIV | |
| | LDTNYISEKINININDLSIRYVWSNDGSDFILMSTDEENK | |
| | VSQVKIRFTNVFKGNTISDKISFNFSDKQDISINKIISTF | |
| | TPSYYVEGLLNYDLGLISLYNEKFYINNLGMMVSGLVYIN | |
| | DSLYYFKPPIKNLITGFTTIGDDKYYFNPDNGGAASVGET | |
| | IIDGKNYYFSQNGVLQTGVFSTEDGFKYFAPADTLDENLE | |
| | GEAIDFTGKLIIDENVYYFGDNYRAAIEWQTLDDEVYYFS | |
| | TDTGRAFKGLNQIGDDKFYFNSDGIMQKGFVNINDKTFYF | |
| | DDSGVMKSGYTEIDGKYFYFAENGEMQIGVFNTADGFKYF | |
| | AHHDEDLGNEEGEALSYSGILNFNNKIYYFDDSFTAVVGW | |
| | KDLEDGSKYYFDENTAEASIGISIINDGKYYFNDSGIMQI | |
| | GFVTINNEVFYFSDSGIVESGMQNIDDNYFYISENGLVQI | |
| | GVFDTSDGYKYFAPANTVNDNIYGQAVEYSGLVRVNEDVY | |
| | SFGESYTIETGWIYDSENESDKYYFDPETKKAYKGINVID | |
| | DIKYYFDENGIMRTGLITFEDNHYYFNEDGEMQYGYLNIE | |
| | DKMFYFSEDGIMQIGVFNTPDGFKYFAHQNTLDENFEGES | |
| | INYTGWLDLDEKRYYFTDEYIAATGSVIIDGEEYYFDPDT | |
| | AQLVISE | |
| TcdB8_ES130 | MSLVNRKQLEKMANVRFRTQEDEYVAILDALEEYHNMSEN | 39 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTIVESATNDTLESFREN | |
| | LNDPEFNHTAFFRKRMQIIYDKQQNFINYYKAQKEENPDL | |
| | IIDDTVKSYLSDEYSKDIDELNAYIEESLNKIAENSGNDV | |
| | RNFEEFKDGEVFNLYEQELVERWNLAAASDILRVAILKNI | |
| | GGVYLDVDMLPGIHPDLFKNINKPDSVKTAVDWEEMKLEA | |
| | IMKYKEYIPEYTSKHFDTLDEEVQSSFESVLASKSDKSEI | |
| | FLPLGDIEVSPLEVKIAFAKGSIINQALISVKDSYCSDLL | |
| | IKQIQNRYKILNDTLGPIISQGNDFNTTMNSFGESLGAIS | |
| | SEDNISFIAKIGSYLRVGFYPEANTTITLSGPTVYAGAYK | |
| | DLLTFKEISLDTSILTSELRNFEFPKDNISQATEQEKNSL | |
| | WQFNEERAKIQFEEYKRAYFEGALGEDDNLDFSQNTVTDK | |
| | EYLLEKISSSTKSSERGYVHYIVQLQGDKISYEAACNLFA | |
| | KNPYDSILFQKNIEDSEIAYYYNPADGEIQEIDKYRIPDR | |
| | ISDRPKIKLTFIGHGKDEFNTDIFAGLDVDSLSTEIETAI | |
| | DLAKEDISSKSIEINLLGCNMFSYSINVEETYPGKLLLKV | |
| | KDKISELMPSISQDSIIVSANQYEVRINSEGRRELLDHSG | |
| | EWINKEESIIKDISSKEYISFNPKENKITVKSKNLPELST | |
| | LLQEIRNNSNLSDIELEEKVMLAECEINVISNIDTQIVEE | |
| | RIEEAKNLTSDSINYIKNEFKLIESISDSLYDLKQQNELD | |
| | DSHFISFEDISKTEDGFSIRFINKETGESIFVETEKEIFS | |
| | EYANHIEREISNIKDTIFDTVNGKLVKKVNLDAIHEVNTL | |
| | NAAFFIQSLIGYSSSKESLSNLSVAMKVQVYAQLFSTGLN | |
| | TITDAAKVVELVSTALDETIDLLPTLSEGLPVIATIIDGV | |
| | SLGAAIKELSETSDPLLRQEIEAKIGIMAVNLTAATTAII | |
| | TSSLGIASGFSILLVPLAGISAGIPSLVNNELVLRDKATK | |
| | VVDYFKHISLVETEGAFTLLDDKIMIPQDDLVISEIDFNN | |
| | NSIVLGKCEIWRMEGGSGHTVTNDIDHFFSSPTITYIKPH | |
| | LSIYDVLEVQKEELDLSKDLMVLPNAPNRVFAWETGWTPG | |
| | LRSLENEGTKLLDRIRDHYKGEFYWRYFAFIADALITTLK | |
| | PRYEDTNIRINLDSNNRSFIVPIITTEHIREKLSYSFHGS | |
| | GGTYALSLSQYNMGINIELSESDVWIIDVDNVVRDVTIDS | |
| | DKIKKGDLIEGILSTLSIEDNKIILNHHEINFSGDVNGSN | |
| | GFISLTFSILEGINAHIEVDLLSKSYKLLISGELKILMLN | |
| | SNHIQQKIDYIGFNSELQKNIPYSFVDSEGKENGFINGST | |
| | KEGLFVSELPDVVLISKVYMDDSKPSFGYYSNNLKDVKVI | |
| | TKDNVNILTGYYLKDDIKISLSFTLQDEKTIKLNGVHLDE | |
| | SGVAEILKFMNKKGSTNTSDSLMSFLESVNIKSIFVNFLQ | |
| | SKINFILDANFIISGTTSIGQFEFICDENDNIQPYFIKFN | |
| | TLETTYTLYVGNRQNMIVEPNYDLDDSGDISSTVINFSQK | |
| | YLYGIDSCVNKVVISPNIYTDEINITPVYETNNNYPEVIV | |
| | LDANYINEKINVNINDLSIRYVWSNDGNDFILMSTSEENK | |
| | VSQVKIRFVNVFKDKTLANKLSFNFSDKQDVPVSEIISAF | |
| | TPSYYEDGLIGYDLGLVSLYNEKFYINNFGMMVSGLIYIN | |
| | DSLYYFKPPVNNLITGFVTVGDDKYYFNPTNGGAASIGET | |
| | IIDDKNYYFNQSGILQTGVFSTEDGLKYFAPANTLDENLE | |
| | GEAIDFTGKLIIDENIYYFEDNYRGAVEWKELDGEMYYFS | |
| | PETGKAFKGLNQIGDDKYYFNSDGIMKKGFVSINDKKYYF | |
| | DDSGVMKVGYIEIDGKYFYFAENGEMQIGVENTSDGFKYF | |
| | AHHNEDLGNEEGEAISYSGILNFNNKIYYFDYSFTAVVGW | |
| | KDLEDGSKYYFDEDTAEAYVGLSLINDGQYYFNDDGIMQV | |
| | GFVTINNKVFYFSDSGIIESGVQNIDDNYFYIDEKGIVQI | |
| | GVFDTSDGYKYFAPANTVNDNIYGQAVDYSGLVRVGEDIY | |
| | YFGETYTIETGWIYDMENESDKYYFNPETKKACKGINLID | |
| | DIKYYFDENGIMRTGLISFENNDYYFNENGEMHFGYINIE | |
| | DKMFYFGEDGVMQIGVFNTPDGFKYFAHQNTLDENFEGES | |
| | INYTGWLDLEGKRYYFTDEYIAATGTVTIDGEEYYFDPDT | |
| | AELVVSE | |
| TcdB9_SE844 | MSLVNRKQLEKMANVRFRTQEDEYVAILDALEEYHNMSEN | 40 |
| | TVVEKYLKLKDINSLTDIYIDTYKKSGRNKALKKFKEYLV | |
| | TEVLELKNNNLTPVEKNLHFVWIGGQINDTAINYINQWKD | |
| | VNSDYNVNVFYDSNAFLINTLKKTIVESATNDTLESFREN | |
| | LNDPRFDYNKFYRKRMEIIYDKQKNFINYYKTQREENPDL | |
| | IIDDIVKIYLSNEYSKDIDELNSYIEESLNKVTENSGNDV | |
| | RNFEEFKGGESFKLYEQELVERWNLAAASDILRISALKEV | |
| | GGVYLDVDMLPGIQPDLFESIEKPSSVTVDFWEMVKLEAI | |
| | MKYKEYIPGYTSEHFDMLDEEVQSSFESVLASKSDKSEIF | |
| | SSLGDMEASPLEVKIAFNSKGIINQGLISVKDSYCSNLIV | |
| | KQIENRYKILNNSLNPAISEDNDFNTTTNAFIDSIMAEAN | |
| | ADNGREMMELGKYLRVGFFPDVKTTINLSGPEAYAAAYQD | |
| | LLMFKEGSMNIHLIEADLRNFEISKTNISQSTEQEMASLW | |
| | SFDDARAKAQFEEYKKNYFEGSLGEDDNLDFSQNTVVDKE | |
| | YLLEKISSLARSSERGYIHYIVQLQGDKISYEAACNLFAK | |
| | TPYDSVLFQKNIEDSEIAYYYNPGDGEIQEIDKYKIPSII | |
| | SDRPKIKLTFIGHGKDEFNTDIFAGLDVDSLSTEIETAID | |
| | LAKEDISPKSIEINLLGQNMFSYSINVEETYPGKLLLKVK | |
| | DKISELMPSISQDSIIVSANQYEVRINSEGRRELLDHSGE | |
| | WINKEESIIKDISSKEYISFNPKENKIIVKSKNLPELSTL | |
| | LQEIRNNSNSSDIELEEKVMLAECEINVISNIDTQVVEER | |
| | IEEAKSLTSDSINYIKNEFKLIESISDALYDLKQQNELEE | |
| | SHFISFEDISETDEGFSIRFIDKETGESIFVETEKAIFSE | |
| | YANHITEEISKLKDTIFDTVNGKLVKKVNLDATHEVNTLN | |
| | AAFFIQSLIEYNSSKESLSNLSVAMKVQVYAQLFSTGLNT | |
| | ITDAAKVVELVSTALDETIDLLPTLSEGLPVIATIIDGVS | |
| | LGAAIKELSETSDPLLRQEIEAKIGIMAVNLTTATTAIIT | |
| | SSLGIASGFSILLVPLAGISAGIPSLVNNELVLRDKATKV | |
| | VDYFKHVSLVETEGVFTLLDDKIMMPQDDLVISEIDFNNN | |
| | SIVLGKCEIWRMEGGSGHTITDDIDHFFSAPSITYREPHL | |
| | SIYDVLEVQKEELDLSKDLMVLPNAPNRVFAWETGWTPGL | |
| | RSLENDGTKLLDRIRDNYEGEFYWRYFAFIADALITTLKP | |
| | RYEDTNIRINLDSNTRSFIVPIITTEYIREKLSYSFYGSG | |
| | GTYALSLSQYNMGINIELSESDVWIIDVDNVVRDVTIESD | |
| | KIKKGDLIEGILSTLSIEENKIILNSHEINFSGDVNGSNG | |
| | FVSLTFSILEGINAIIEVDLLSKSYKLLISGELKILMLNS | |
| | NHIQQKIDYIGFNSELQKNIPYSFVDSEGKENGFINGSTK | |
| | EGLFVSELPDVVLISKVYMDDSKPSFGYYSNNLKDVKVIT | |
| | KDNVNILTSYYLKDDIKISLSLTLQDEKTIKLNSVHLDES | |
| | GVAEILKFMNRKGSTNTSDSLMSFLESMNIKSIFVNFLQS | |
| | NIKFILDANFIISGTTSIGQFEFICDENDNIQPYFIKFNT | |
| | LETNYTLYVGNRQNMIVEPNYDLDDSGDISSTVINFSQKY | |
| | LYGIDSCVNKVVISPNIYTDEINITPVYEANNTYPEVIVL | |
| | DTNYISEKINININDLSIRYVWSNDGSDFILMSTDEENKV | |
| | SQVKIRFTNVFKGNTISDKISFNFSDKQDVSINKIISTFT | |
| | PSYYVEGLLNYDLGLISLYNEKFYINNFGMMVSGLVYIND | |
| | SLYYFKPPIKNLITGFTTIGDDKYYFNPDNGGAASVGETI | |
| | IDGKNYYFSQNGVLQTGVFSTEDGFKYFAPADTLDENLEG | |
| | EAIDFTGKLTIDENVYYFGDNYRAAIEWQTLDDEVYYFST | |
| | DTGRAFKGLNQIGDDKFYFNSDGIMQKGFVNINDKTFYFD | |
| | DSGVMKSGYTEIDGKYFYFAENGEMQIGVFNTADGFKYFA | |
| | HHDEDLGNEEGEALSYSGILNFNNKIYYFDDSFTAVVGWK | |
| | DLEDGSKYYFDENTAEASIGISIINDGKYYFNDSGIMQIG | |
| | FVTINNEVFYFSDSGIVESGMQNIDDNYFYISENGLVQIG | |
| | VFDTSDGYKYFAPANTVNDNIYGQAVEYSGLVRVNEDVYS | |
| | FGESYTIETGWIYDSENESDKYYFDPETKKAYKGINVIDD | |
| | IKYYFDENGIMRTGLITFEDNHYYFNEDGIMQYGYLNIED | |
| | KTFYFSEDGIMQIGVFNTPDGFKYFAHQNTLDENFEGESI | |
| | NYTGWLDLDEKRYYFTDEYIAATGSVIIDGEEYYFDPDTA | |
| | ELVISE | |
| TcdB10_CD10- | MSLVNRKQLEKMANVRFRVQEDEYVAILDALEEYHNMSEN | 41 |
| 165 | TVVEKYLKLKDINSLTDTYIDTYKKSGRNKALKKFKEYLV | |
| | TEVLELKSSNVVPVEKNLHFVWIGGKINDTAINYINQWKD | |
| | VNSDYNVNVFYDSNAFLINTLKKTIVDSATNETLESFREN | |
| | LDDPRFDYNKFYRKRMEIIYDKQKNFINYYKAQREENPDF | |
| | IIDDIVKSYLSNEYSKDIDELNAYIEESLNKVKENSGNDI | |
| | RDFEEFKGGNSFNLYEQELVERWNLAAASDILRISALKEV | |
| | GGVYLDVDMLPGIQPDLFESIEKPSSVTVDFWEMVKLEAI | |
| | MKYKEYIPGYTSEHFDILDEEVQSSFESVLASKSDKSEIF | |
| | SSLGDIEASPLEVKIAFNSKGIINQGLISVKDSYCSNLIV | |
| | KQIENRYKILNDSLNPAISEDNDFNTTTNTFIDSIMAEAN | |
| | ADNGRFMMELGKYLRVGFFPDVKTTINLSGPEAYAAAYQD | |
| | LLMFKEYSINIHLLESDLRNFEISKTNISQSTEQEMASLW | |
| | SFDDARAKAQFQEYKRNYFEGALGEDDNLDFSQNTITDKE | |
| | YLIEKISSSAKNSERGYVHYIIQLQGDNISYEAACNLFAK | |
| | NPYDSILFQKNIEDSTIAYYYNPADGEIQEIDKYRIPDRI | |
| | SDRPKIKLTFIGHGKSEFNTDIFANLNVDSLSSEIETAID | |
| | LAKTDISPKAIEINLLGCNMFSYSVNVEETYPGKLLLKVK | |
| | DKVSELMPSISQDSIIVSANQYEVRINSEGRRELLDHSGE | |
| | WINKEESIIKDISSKEYISFNSKENKIIVKSKNLPELSTL | |
| | LQEIRNNSNLSDIELEEKVMLAECEISVVSDIDTQVVEER | |
| | IEEAKNLTSDSINYIKNEFKLIESISDALYDLKQQNELED | |
| | SHFISFEDISETDEGFSIRFIDKETGESIFVETEKTIFSE | |
| | YANHITEEISKVKDTIFDTVNGKLVKKVNLDATHEVNTLN | |
| | AAFFIQSLIGYNSSKESLSNLSVAMKVQVYAQLFSTGLNT | |
| | ITDAAKVVELVSTALDETIDLLPTLSEGLPIIATIIDGVS | |
| | LGASIKELSETSDPLLRQEIEAKIGIMAVNLTAATTAIIT | |
| | SSLGIASGFSILLVPLAGISAGIPSLVNNELILRAEAKNV | |
| | VDYFSHISLAESEGAFTLLDDKIMMPQDDLVISEIDFNSN | |
| | SITLGKCEIWRMEGGSGHTVTNDIDHFFSAPSTTYREPYL | |
| | SIYDVLDVKKEELDLSKDLMVLPNAPDRIFGWERGWTPGL | |
| | RGLENDGTKLLDRIRDHYEGQFYWRFFAFIADSVITKLKP | |
| | RYEDTNIRISLDSNTRSFIVPVITTEYIREKLSYSFYGSG | |
| | GTYALSLSQYNMNINIELNESDTWVIDIDNVVRDVTIESD | |
| | KIKKGDLIENILSKLSIEENKIILDNHEINFSGTLNGGNG | |
| | FVSLTFSILEGINAVIEVDLLSKSYKVLISGELKTLMENS | |
| | NSVQQKMDYIGLNSEVQKNIPYSFTDDKGKENGFINCSTK | |
| | EGLFVSELSDVVLISKVYMDDSKPSSGYYSYDLKDVKVIT | |
| | KDDVIILTGYYLKDDIKISLSFTIQDENTIKLNGVYLDEN | |
| | GVAEILKFMNKKGSTNTSDSLMSFLESMNIKSIFINSLQS | |
| | NTKLILDTNFIISGATSIGQFEFICDKDNNIQPYFIKFNT | |
| | LETKYTLYVGNRQNMIVEPNYNLDDSGDISSTVINFSQKY | |
| | LYGIDSCVNKVIISPNIYTDEINITPVYEANNTYPEVIVL | |
| | DTNYISEKINININDLSIRYVWSNDGSDFILMSTDEENKI | |
| | SQVKIRFTNVFKGNTIADKISFNFSDKQDVSINKIISTFT | |
| | PSYYVEQLLNYDLGLISLYNEKFYINNFGMMVSGLVYIND | |
| | SLYYFKPPIKNLITGFTTIGDDKYYFNPDNGGAASVGETI | |
| | IDGKNYYFSQNGVLQTGVFSTEDGFKYFAPADTLDENLEG | |
| | EAINFTGKLIIDENIYYFGDNYRAAEEWQTLDDEVYYFST | |
| | DTGKAFKGLNQIGDDKFYFNSDGIMQKGFVNINDKTFYFD | |
| | DSGVMKSGYLEIYGKYFYFAENGEMQIGVFNTTDGFKYFA | |
| | HQDEDLGNEEGEALSYSGILNFNNKIYYFDDSFTAIVGWK | |
| | DLEDGSKYYFDENTAEASIGISIINDGKYYFNDSGIMQIG | |
| | FVTINDKVFYFSDSGIVESGMQNIDDNYFYISENGLVQIG | |
| | VFDTSDGYKYFAPANTVNDNIYGQAVEYSGLVKVNEDVYS | |
| | FGESYTIETGWIYDSENESDKYYFDPETKKAYKGINTIDD | |
| | IKYYFDENGIMRTGLITFEDNHYYFNEDGVMQYGYLNIED | |
| | KMFYFNEDGVMQIGVFNTPDGFKYFAHQNTLDENFEGESI | |
| | NYTGWLDLDGKKYYFTEEYIAATGSVTIDDEEYYFDPDTA | |
| | ELVVSE | |
| TcdB11_CD160 | MSLINRKQLEKMANVKFRVQEDEYIAILDALEEYHNMSEN | 42 |
| | TVVEKYLKLKDINSLTETYIDTYKKSGRNKALKKFKEYLV | |
| | TEVLELKNSNVAPVEKNLHFVWIGGKINDTAINYINQWKD | |
| | VNSDYNVNVFYDSNAFLINTLKKTIVDSATNETLESFREN | |
| | LDDPRFDYNKFYRKRMEIIYDKQKNFINYYKAQREENPDL | |
| | IIDDIVKTYLSNEYSKDIDELNAYIEESLSKVTENSGNDV | |
| | RNFEEFKGGESFNLYEQELVERWNLAAASDILRVSALKEV | |
| | GGVYLDVDMLPGIQPDLFESIEKPSSVTVDFWEMVKLEAI | |
| | MKYKEYIPGYTSEHFDMLDEEVQSSFESVLASKSDKSEIF | |
| | SSLGDVESSPLEVKIAFNSKGIINQGLISVKDSYCSNLIV | |
| | KQIENRYKILNNSLNPAISEDNDFNTTTNTFIDSIMAEAN | |
| | ADNSRFMMELGKYLRVGFFPDVKTTINLSGPEAYAAAYQD | |
| | LLMFKEYSMNIHLLESDLRNFEISKTNISQSTEQEMASLW | |
| | SFDDARAKAQFQEYKKNYFEGALGEDDNLDFSENTVLDKD | |
| | YILEKISSSTRSSERGYVHYIVQLQGDKISYEAACNLFAK | |
| | NPYDSILFQNNIEDSEIAYYYNPADGEIQEIDKYRIPDII | |
| | SDRPKVKLTFIGHGKSEFNTDIFANLDVDSLSSEIETVID | |
| | LAKTDISPKAIEINLLGCNMFSYSINVEDTYPGKLLLKVK | |
| | DKVSELLPSINQDSIIVSANQYEVRINSEGRRELLDHSGE | |
| | WINKEESIIKDISSKEYISFNPQENKIIVKSKNLPELSTL | |
| | VQEIRNNSNSGDIELEEKVMLAECEISVVSDIDTQVVEER | |
| | IEEAKNLTSDSINYIKNEFKLIESISDALYDLKQQNELED | |
| | SHFISFEDISETDEGFSIRFIDKETGESIFVETEKTIFSE | |
| | YANHITEEITKVKDTIFDTVNGKLVKKVNLDATHEVNTLN | |
| | AAFFIQSLIGYNSSKESLSNLSVAMKVQVYAQLFSTGLNT | |
| | ITDAAKVVELVSTALDETIDLLPTLSEGLPIIATIIDGVS | |
| | LGASIKELSETSDPLLRQEIEAKIGIMAVNLTAATTAIIT | |
| | SALGIASGFSILLVPLAGISAGVPSLVNNELVLRDKATKV | |
| | VDYFSHISLAESEGAFTLLDDKIMMLQDDLVISEIDFNNN | |
| | SITLGKCEIWRMEGGSGHTVVDDIDHFFSSPPITYREPHL | |
| | SIYDVLEVKKEELDLSKDLMVLPNAPNRVFGWETGWTPGL | |
| | RGLENDGTKLLDRIRDYYEGQFYWRFYAFVADALITTLKP | |
| | RYEDTNVRISLDSNTRSFIVPVITTEYIREKLSYSFYGSG | |
| | GTYALSLSQYNMNINIELNENDTWVIDVDNVVRDVTIESD | |
| | KIKKGDLIENILSKLSIEENKIILQNHEINFSGTLNGGNG | |
| | FVSLTFSILEGINAVIEVDLLSKSYKVLVSGELKTLMLNS | |
| | NSVQQKIDYIGLNSEVQKNIPYSFTDDKGKENGFINCSTK | |
| | EGLFISELSDVVLISKVYMDDSKPSFGYYSDDLKDVKVIT | |
| | KDDVILLTGYYLKDDIKISLSFTIQDENTIKLNGVYLDEN | |
| | GVAEILKFMNKKGSTNTSDSLMSFLESMNIKSIFMNFVQS | |
| | NVKLILDTNFIINGTTSIGQFEFICDKDNNIQPYFIKFNT | |
| | LETKYTLYVGNRENMIVEPNYNLDDSGDISSTVINFSQKY | |
| | LYGIDSCINKVIISPNIYTDEINITPVYEANNTYPEVIVL | |
| | DANYISEKINININDLSIRYVWSNDGSDFILMSTNEEDKV | |
| | SQIKIRFTNVFKGNTMSDKLSFNFSDKQDVSISKIISTFT | |
| | PSYYRENLLNYDLGMISLYNEKFYINNFGMMVSGLVYIND | |
| | SLYYFKPPLKNLITGFTTIGDDKYYFNPDNGGAASVGETI | |
| | IDGKNYYFSQNGVLQTGVFSTEDGFKYFAPANTLDENLEG | |
| | EAIDFTGKLTIDENVYYFGDNYRAAIEWQTLDDEMYYFST | |
| | ETGRAFKGLNQIGDDKFYFNSSGIMQKGFVNINDKTFYFD | |
| | DSGVMKSGYIEIDGKYFYFAENGEMQIGVFNTTDGFKYFA | |
| | HQDEDLGNEEGEALSYSGILNFNNKIYYFDDSFTAVVGWK | |
| | DLEDGSKYYFDEDTAEAYIGISTINDGQYYFNDSGIMQIG | |
| | FVTINDKVFYFSDSGIVESGMQNIDDNYFYIDDNGLVQIG | |
| | VFDTSDGYKYFAPANTVNDNIYGQAVECSGLVRVGEDVYC | |
| | FGESYTIETGWIYDMENESDKYYFDSETKKAYKGINVIDD | |
| | IKYYFDENGIMRTGLISFENNHYYFNADGEMQYGYLNIED | |
| | KMFYFSEDGFMQIGVFNTPDGFKYFAHQSTLDENFEGESI | |
| | NYTGWLDLDDKKYYFTDEYIAATGSVIIDDEEYYFDPETA | |
| | ELVVSE | |
| TcdB12_173070 | MSLVNRKQLEKMANVKFRTQEDEYVAILDALEEYHNMSEN | 43 |
| | TVVEKYLKLKDINSLTDAYIDTYKKSGRNKALKKFKEYLT | |
| | TEVIELKNSNLTPVEKNLHFVWIGGQISDTAINYINQWKD | |
| | VNSDYNTNVFYDSNAFLINTLKKTIVEATTNDTLESFSEN | |
| | LNDPRFDHNNFYRKRMEMIYDKQKNFINYYKAQREENPEL | |
| | IIDDIVKTYLSNEYSKEIDELNAYIEESLNKITQNSGNDV | |
| | RNFEEFKNGESFKLYEQELVERWNLAAASDILRISALKEI | |
| | GGVYLDVDMLPGIQPDLFESIEKPSSVTVDFWEMTKLEAI | |
| | MKYKEYIPGYTSEHFDMLDEEVQSSFESALASKSDKSEIF | |
| | SSLGDMEASPLEVKIAFNSKGIINQGLISVKDSYCSNLIV | |
| | KQIENRYKILNNSLNPAISEDNDFNTTTNTFIDSIMAEAN | |
| | ADNGRFMMELGKYLRVGFFPDVKTTINLSGPEAYAAAYQD | |
| | LLMFKEDSMNIHLIEADLRNFEISKTNISQSTEQEMASLW | |
| | SFDDARAKAQFEEYKRNYFEGALGEDDNLDFSQNTVVDKE | |
| | YLLEKISSLARSSERGYIHYIVQLQGDKISYEAACNLFAK | |
| | TPYDSILFQKNIEDSEIAYYYNPGDDEIQEIDKYRIPSII | |
| | SDRPKIKLTFIGHGKDEFNTDIFAGLDVDSLSTEIETVID | |
| | LAKEDISPKAIEINLLGQNMFSYSINIEETYPGKLLLKVK | |
| | DKISELMPSISQDSIIVSANQYEVRINSEGRRELLDHSGE | |
| | WINKEESIIKDISSKEYISFNSKENKIIVKSKNLPELSTL | |
| | LQEIRNNSNSSDIELEEKVMLAECEINVISNIDTQIVEER | |
| | IEEAKNLTSDSINYIKNEFKLIESISDALCDLKQQNELED | |
| | SHFISFEDISETDEGFSIRFINKETGESIFVETEKTIFSE | |
| | YANHITEEISKVKDTIFDTVNGKLVKKVNLDTTHEVNTLN | |
| | AAFFIQSLIEYNSSKESLSNLSVAMKVQVYAQLFSTGLNT | |
| | ITDAAKVVELVSTALDETIDLLPTLSEGLPVIATIIDGVS | |
| | LGSAIKELSETSDPLLRQEIEAKIGIMAVNLTAATTAIIT | |
| | SALGIASGFSILLVPLAGISAGIPSLVNNELILRDKATKV | |
| | VDYFKHISLAETEGAFTLLDDKIIMPQDDLVISEIDFNNN | |
| | SITLGKCEIWRMEGGSGHTVTDDIDHFFSSPSITYREPYL | |
| | SIYDVLEVQKEELDLSKDLMVLPNAPNRVFAWETGWTPGL | |
| | RSLENDGTKLLDRIRNHYEGEFYWRYFAFIADALITTLKP | |
| | RYEDTNIRINLDSNTRSFVVPVITTEYIRENLSYSFYGSG | |
| | GTYALSLSQYNMGINIELSESDVWIIDVDNVVRDVTIESD | |
| | KIKKGDLIEGILSTLSIEDNKIILNSHELNFSGDVNGSNG | |
| | FVSLTFSILEGINAIIEVDLLSKSYKLLLSGELKTLMSNS | |
| | NYIQQKIDYIGFNSELQKNIPYSFVDAEGKKNGFINVSTK | |
| | EGLFASELSDVVLISKVYMDNSKPSFGHYSDILKDVKVIT | |
| | KDDINILTGYYLKDDIKISLSFTLQDEHTIKLNGVHLDEK | |
| | GVAEILTFMNKKVGTNTSDSLMSFLKSMNINNVFSHSLQD | |
| | KVNLVLETNFIISGMTSIGQFEFICDENDNIQPYFIKFNA | |
| | LDTKYTLYLGNRQNMIVEPNYDLDDSGNISSTVINFSQKH | |
| | LYGIDSFINKVISPNLYTDEINITPVHETNNTYPEVIVLD | |
| | ANYISEKIKVNINDLSIRYIWSNDGNDFILMSTIGEDKAS | |
| | QVKIRFANVFKGNTLANKLSFNFSDKQDVSLSEIISAFTP | |
| | SKYEDGFSSYKLGLISFYNEKFYINNFGMKVSGLIYINDS | |
| | LYYFKPPVNNLITGFTTVGDDKYYFNPTNGGAASIGDTII | |
| | DDKNYYFNQIGVLQTGVFSTEDGFKYFAPANTLDENLEGE | |
| | AIDFTGKLIIDENIYYFEDNYRGAVEWKELDGEMYYFSPE | |
| | TGKAFKGLNQIGDDKYYFNSDGIMQKGFVSINDKKHYFDD | |
| | SGVMKVGYTEIDGKYFYFAENGEMQIGVFNTSDGFKYFAH | |
| | YNEDLGNEEGEALSYSGILNFNNKIYYFDDSFTAVVGWRN | |
| | LDDGSKYYFDENTAEAFIGFSLINDEQYYFNEDGIMQVGF | |
| | VTINDRVFYFSDSGIIESGVQNIDDNYFYIDEKGIVQIGV | |
| | FDTSDGYKYFAPPNTVNENIYGQAVEYSGLVKVNEDVYYF | |
| | GETYLIETGWIYDMENESDKYYFDPETKKAYKGINVINDT | |
| | KYYFDENGIMRTGLISFENNHYYFNEDGVMQSGYINIEDK | |
| | MFYFSEDGIMQIGVFNTPDGFKYFAHQNTLDQNFEGESIN | |
| | YTGWLDFNEKRYYFTDEYIAATGSVTIDDEEYYFDPDTAE | |
| | LVLSE | |

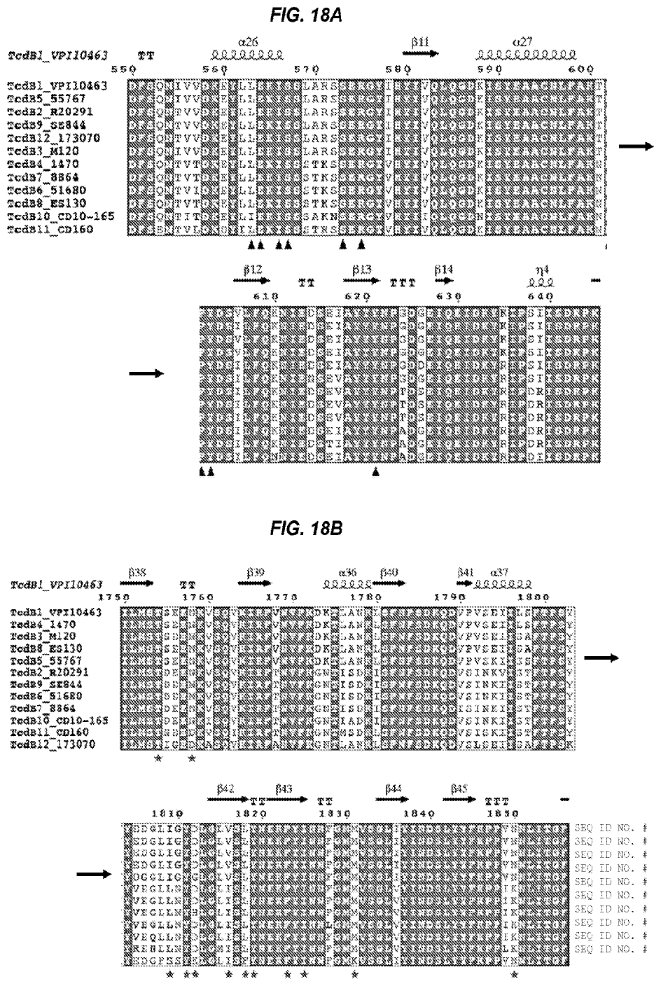

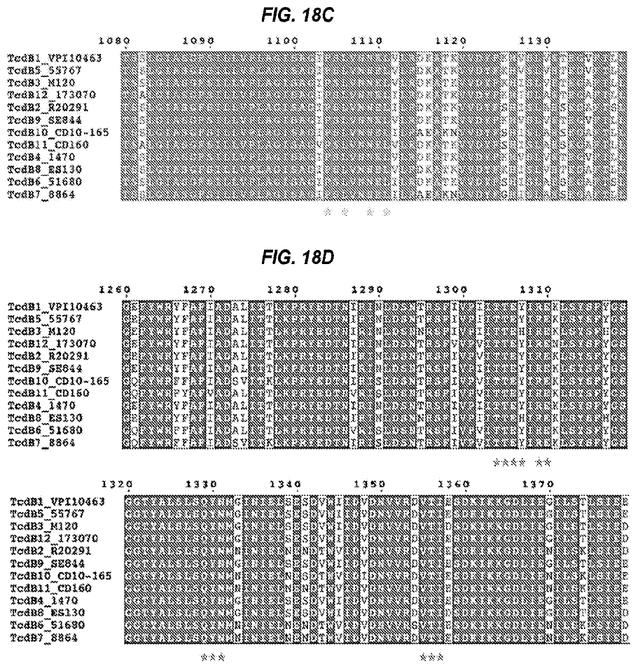

In some embodiments, the neutralizing receptor decoy antibody (RDA) is capable of neutralizing most and/or all TcdB subtypes. In some embodiments, the RDA is capable of neutralizing all TcdB subtypes with conserved CSPG4-binding sites (non-limiting examples shown in FIG. 18A-18D). Non-limiting examples of TcdB subtypes that can be neutralized by the RDA include but are not limited to TcdB1 and TcdB2 (for more examples see Table 7). In some embodiments, the RDA is capable of neutralizing all TcdB subtypes with conserved FZD-binding sites. In some embodiments, the RDA is capable of neutralizing most TcdB subtypes with conserved CSPG4 and/or FZD binding sites. In some embodiments, the RDA is capable of neutralizing most TcdB subtypes with highly conserved CSPG4 and/or FZD binding sites. In some embodiments, the RDA is capable of neutralizing most TcdB subtypes with generally conserved CSPG4 and/or FZD binding sites.

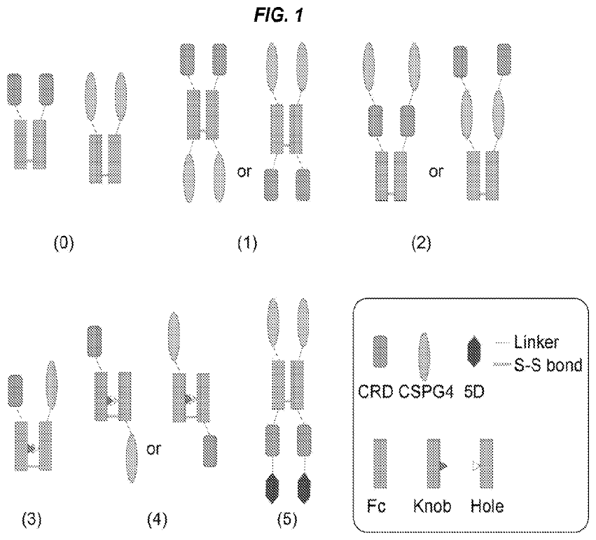

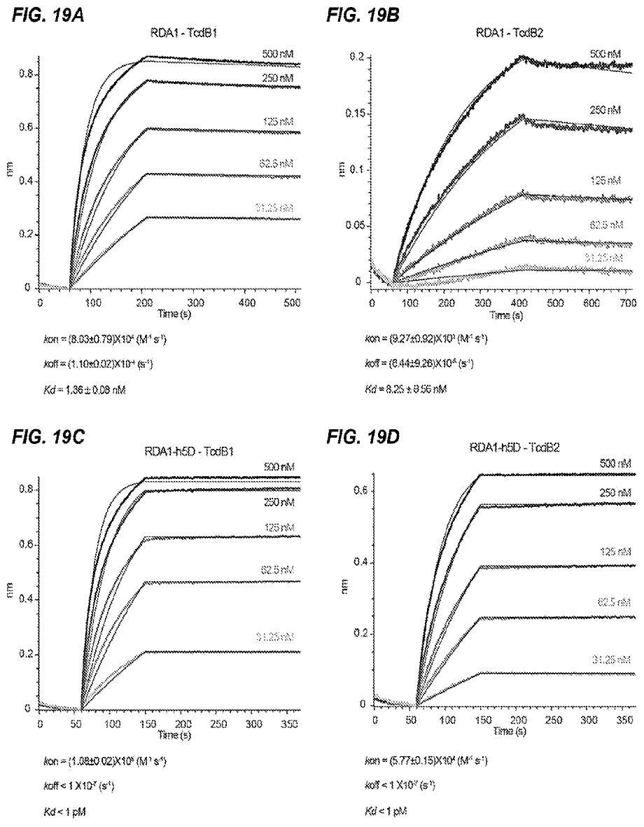

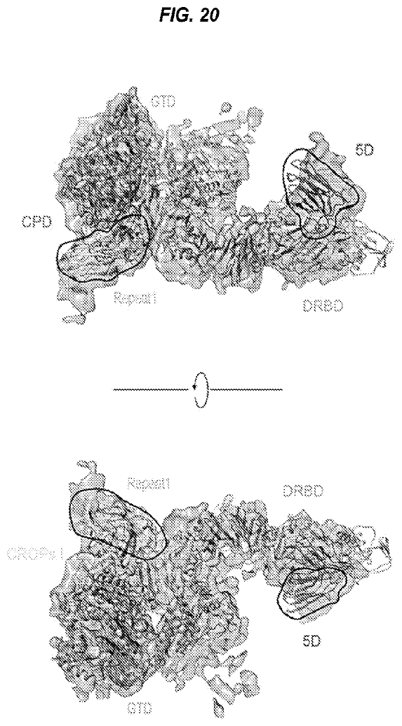

Referring now to FIGS. 1-20, the present invention features a neutralizing receptor decoy antibody (RDA) for use in the prevention and treatment of Clostridium difficile infection (CDI). In some embodiments, the present invention may feature a broad-spectrum neutralizing composition comprising a neutralizing receptor decoy antibody (RDA) that neutralizes a toxin of Clostridium difficile in various strains.

TABLE 8
Non-limiting examples of a receptor decoy
antibody (RDA) composition as described herein:

| | | SEQ |
|---|---|---|
| Name | Sequence | ID NO: |
| RDA1 | HHHHHHHHIEGRPASGSLPEPCVPE | 46 |
| | PGLPPVFANFTQLLTISPLVVAEGG | |
| | TAWLEWRHVQPTLDLMEAELRKSQV | |
| | LFSVTRGARHGELELDIPGAQARKM | |
| | FTLLDVVNRKARFIHDGSEDTSDQL | |
| | VLEVSVTARVPMPSCLRRGQTYLLP | |
| | IQVNPVNDSRTHTCPPCPAPELLGG | |
| | PSVFLFPPKPKDTLMISRTPEVTCV | |
| | VVDVSHEDPEVKFNWYVDGVEVHNA | |
| | KTKPREEQYNSTYRVVSVLTVLHQD | |
| | WLNGKEYKCKVSNKALPAPIEKTIS | |
| | KAKGQPREPQVYTLPPSRDELTKNQ | |
| | VSLTCLVKGFYPSDIAVEWESNGQP | |
| | ENNYKTTPPVLDSDGSFFLYSKLTV | |
| | DKSRWQQGNVFSCSVMHEALHNHYT | |
| | QKSLSLSPGKGGGGSGGGGSGGGGG | |
| | GGGSVDQYNGERGISIPDHGYCQPI | |
| | SIPLCTDIAYNQTIMPNLLGHTNQE | |
| | DAGLEVHQFYPLVKVQCSAELKFFL | |
| | CSMYAPVCTVLEQALPPCRSLCERA | |
| | RQGCEALMNKFGFQWPDTLKCEKFP | |
| | VHGAGELCVGQNTSDKG | |
| RDA1-h5D | HHHHHHHHIEGRPASGSELPEPCVP | 47 |
| | EPGLPPVFANFTQLLTISPLVVAEG | |
| | GTAWLEWRHVQPTLDLMEAELRKSQ | |
| | VLFSVTRGARHGELELDIPGAQARK | |
| | MFTLLDVVNRKARFIHDGSEDTSDQ | |
| | LVLEVSVTARVPMPSCLRRGQTYLL | |
| | PIQVNPVNDSRTHTCPPCPAPELLG | |
| | GPSVFLFPPKPKDTLMISRTPEVTC | |
| | VVVDVSHEDPEVKFNWYVDGVEVHN | |
| | AKTKPREEQYNSTYRVVSVLTVLHQ | |
| | DWLNGKEYKCKVSNKALPAPIEKTI | |
| | SKAKGQPREPQVYTLPPSRDELTKN | |
| | QVSLTCLVKGFYPSDIAVEWESNGQ | |
| | PENNYKTTPPVLDSDGSFFLYSKLT | |
| | VDKSRWQQGNVFSCSVMHEALHNHY | |
| | TQKSLSLSPGKGGGGSGGGGSGGGG | |
| | SGGGGSVDQYNGERGISIPDHGYCQ | |
| | PISIPLCTDIAYNQTIMPNLLGHTN | |
| | QEDAGLEVHQFYPLVKVQCSAELKF | |
| | FLCSMYAPVCTVLEQALPPCRSLCE | |
| | RARQGCEALMNKFGFQWPDTLKCEK | |
| | FPVHGAGELCVGQNTSDKGQVQLVE | |
| | SGGGLVQPGGSLRLSCAASGFTLDY | |
| | YGIGWFRQAPGQGLEAVSYISASAR | |
| | TILYADSVKGRFTISRDNSKNTLYL | |
| | QMNSLRAEDTAVYYCARRRFSASSV | |
| | NRWLADDYDVWGQGTLVTVSS | |

In some embodiments, the present invention features a broad-spectrum neutralizing composition comprising a neutralizing receptor decoy antibody (RDA) that neutralizes a toxin of Clostridium difficile (C. difficile) in various strains of C. difficile. In some embodiments, the RDA comprises a fusion protein comprising a fragment of a Fc region; and a fragment of a chondroitin sulfate proteoglycan 4 (CSPG4) receptor tandemly attached to the Fc region. In other embodiments, the RDA comprises a fusion protein comprising a Fc region fragment, a fragment of a chondroitin sulfate proteoglycan 4 (CSPG4) receptor, and a fragment of frizzled protein (FZD) receptor. In further embodiments, the RDA comprises a fusion protein comprising a Fc region fragment and a fragment of a chondroitin sulfate proteoglycan 4 (CSPG4) receptor, a fragment of frizzled protein (FZD) receptor, and a VHH nanobody.

In some embodiments, the neutralizing receptor decoy antibody (RDA) composition design may be a mono-specific fusion protein comprising a fragment of a fragment crystallizable region (Fc region) and a fragment of CSPG4. In other embodiments, the neutralizing receptor decoy antibody (RDA) composition design may be a mono-specific fusion protein comprising a fragment of a cysteine rich domain (CRD) of frizzled proteins (FZDs) (see FIG. 1 (0)). In some embodiments, the neutralizing receptor decoy antibody (RDA) composition design may be a bi-specific fusion protein comprising a fragment of a fragment crystallizable region (Fc region), a fragment of CSPG4, and a fragment of a cysteine rich domain (CRD) of frizzled proteins (FZDs) (See FIGS. 1 (1), (2), (3), and (4)). In further embodiments the neutralizing receptor decoy antibody (RDA) composition design may be a tri-specific fusion protein comprising a fragment of a fragment crystallizable region (Fc region), a fragment of CSPG4, a fragment of a cysteine rich domain (CRD) of frizzled proteins (FZDs), and a VHH nanobody (See FIG. 1 (5)).

In some embodiments, the neutralizing receptor decoy antibody (RDA) composition design may comprise a fusion protein comprising a fragment of a Fc region and a fragment of CSPG4 and/or the cysteine rich domain (CRD) of frizzled proteins (FZDs), respectively. In some embodiments, the RDA design is a hornodimer that has a CRD at the N-terminus and a CSPG4 at the C-terminus or vice versa. In some embodiments, the RDA design is a hornodimer that has a CSPG4 and CRD tandemly fused to Fc. In some embodiments, the RDA design is a heterodimer with both CSPG4 and CRD at the N-terminus. In some embodiments, the RDA design is a heterodimer with a CRD at the N-terminus and a CSPG4 at the C-terminus or vice versa (FIG. 1).

In some embodiments, the RDA composition may comprise a fusion protein comprising a fragment of a Fc region and a fragment of a chondroitin sulfate proteoglycan 4 (CSPG4) receptor tandemly attached to the Fc region. In some embodiments, the fragment of the CSPG4 receptor is tandemly attached to the N-terminal of the Fc region. In other embodiments, the fragment of the CSPG4 receptor is tandemly attached to the C-terminal of the Fc region. In further embodiment, the fragment of the CSPG4 receptor is tandemly attached to both the N- and C-terminal of the fragment of the Fc region.

In some embodiments, the RDA composition may comprise a fusion protein comprising a fragment crystallizable region (Fc region) fragment, a fragment of a chondroitin sulfate proteoglycan 4 (CSPG4) receptor, and a fragment of frizzled protein (FZD) receptor. In some embodiments, both the fragment of the FZD receptor and the fragment of the CSPG4 receptor are tandemly attached to the Fc region, such that the CSPG4 receptor fragment and the FZD receptor fragment are on opposite sides of the Fc region (See FIG. 1). In some embodiments, the CSPG4 receptor fragment is tandemly attached to the Irl-terminal of the Fc region and the FZD receptor fragment C-terminal of the Fc region, or vice versa. In other embodiments, the CSPG4 receptor fragment is tandemly attached to the C-terminal of the Fc region and the FZD receptor fragment N-terminal of the Fc region, or vice versa.

In some embodiments, the CSPG4 receptor fragment tandemly attached to the Fc region and the fragment of the FZD receptor is tandemly attached to the CSPG4 receptor fragment. In some embodiments, the CSPG4 receptor fragment tandemly attached to the N-terminal of the Fc region and the fragment of the FZD receptor is tandemly attached to the CSPG4 receptor fragment. In other embodiments, the CSPG4 receptor tandemly attached to the C-terminal of the Fc region and the fragment of the FZD receptor is tandemly attached to the CSPG4 receptor fragment. In further embodiments, the CSPG4 receptor fragment tandemly attached to the N- or C-terminal of the Fc region and the fragment of the FZD receptor is tandemly attached to the C-terminus of the CSPG4 receptor fragment.

In some embodiments, the FZD receptor fragment tandemly attached to the Fc region and the fragment of the CSPG4 receptor is tandemly attached to the FZD receptor fragment. In some embodiments, the FZD receptor fragment tandemly attached to the N-terminal of the Fc region and the fragment of the CSPG4 receptor is tandemly attached to the FZD receptor fragment. In other embodiments, the FZD receptor tandemly attached to the C-terminal of the Fc region and the fragment of the CSPG4 receptor is tandemly attached to the FZD receptor fragment. In further embodiments, the FZD receptor fragment tandemly attached to the N- or C-terminal of the Fc region and the fragment of the CSPG4 receptor is tandernly attached to the C-terminus of the FZD receptor fragment.

In some embodiments, the RDA composition may comprise a fusion protein comprising a fragment of a Fc region, a fragment of a chondroitin sulfate proteoglycan 4 (CSPG4) receptor, a fragment of frizzled protein (FZD) receptor, and a VHH nanobody. In some embodiments, the CSPG4 receptor fragment is tandemly attached to the N-terminal of the Fc region, and the VHH nanobody is tandemly attached to the CSPG4 receptor fragment and the FZD receptor fragment C-terminal of the Fc region, or vice versa (i.e., the CSPG4 receptor fragment is tandemly attached to the C-terminal of the Fc region and the VHH nanobody is tandemly attached to the CSPG4 receptor fragment, and the FZD receptor fragment is tandemly attached to the N-terminal of the Fc region). In other embodiments, the CSPG4 receptor fragment is tandemly attached to the N-terminal of the Fc region and the FZD receptor fragment N-terminal of the Fc region and the VHH nanobody is tandemly attached to the FZD receptor fragment, or vice versa (i.e., the CSPG4 receptor fragment is tandemly attached to the C-terminal of the Fc region and the FZD receptor fragment is tandemly attached to the N-terminal of the Fc region and the VHH nanobody is tandemly attached to the FZD receptor fragment).

In some embodiments, the CSPG4 receptor fragment tandemly attached to the N-terminal of the Fc region and the fragment of the FZD receptor is tandemly attached to the CSPG4 receptor fragment and the VHH nanobody is tandemly attached to the C-terminal of the Fc region or vice versa. In other embodiments, the CSPG4 receptor tandemly attached to the C-terminal of the Fc region and the fragment of the FZD receptor is tandemly attached to the CSPG4 receptor fragment and the VHH nanobody is tandemly attached to the N-terminal of the Fc region. In some embodiments, the FZD receptor fragment is tandemly attached to the N-terminal of the Fc region and the fragment of the CSPG4 receptor is tandemly attached to the FZD receptor fragment and the VHH nanobody is tandemly attached to the C-terminal of the Fc region. In other embodiments, the FZD receptor is tandemly attached to the C-terminal of the Fc region and the fragment of the CSPG4 receptor is tandemly attached to the FZD receptor fragment and the VHH nanobody is tandemly attached to the N-terminal of the Fc region. In further embodiments, the VHH nanobody is tandemly attached to the N- or C-terminal of the Fc region.

In some embodiments, the CSPG4 receptor fragment, the FZD receptor fragment, and the VHH nanobody may all be linearly attached such that all three fragments are attached to the N- or C- terminal of the Fc region. For example, the VHH nanobody may be tandemly attached to the FZD receptor fragment which is tandemly attached to the CSPG4 receptor fragment which is tandemly attached to the Fc region. The present invention is not limited to the configurations/designs outlined in either FIG. 1 or as described herein. One of ordinary skill in the art would recognize that the CSPG4 receptor fragment, the FZD receptor fragment, or the VHH nanobody could be attached to the Fc region in various configurations.

Without wishing to limit the present invention to any theories or mechanisms it is believed that a tri-specific RDA molecule allows for the composition to have a high specificity and high affinity (i.e., very low K_{D} e.g, <1 pm) for a TcdB toxin.

In some embodiments, the heterodimer RDAs utilize a knobs-into-holes (KiH) strategy. In some embodiments, a CH3 interface is generated favoring a heterodimeric assembly by replacing Thr366 on one CH3 interface with Trp (T366W) to generate a knob. In some embodiments, larger side chains on the other CH3 domain are replaced with smaller ones to generate a hole (e.g. T3663, L368A, Y407V). The present invention is not limited to the above-mentioned method to create a Fc heterodimer.

In some embodiments, the RDA composition described herein is able to neutralize a toxin of C. difficile. In some embodiments, the RDA composition neutralizes the TedB1 toxin. In other embodiments, the RDA composition neutralizes the TcdB2 toxin. Other non-limiting examples of TcdB subtypes the RDA composition can neutralize to include but are not limited to Tcd63, TcdB4, TcdB5, TcdB6, TcdB7, TcdB8, TcdB9, TcdB10, TcdB11, or TcdB12 (see FIG. 18A-18D, and Table 7).

In some embodiments, the RDA mimics a chondroitin sulfate proteoglycan 4 (CSPG4) receptor. In some embodiments, the RDA mimics a frizzled protein (FZD) receptor. In other embodiments, the RDA mimics both a chondroitin sulfate proteoglycan 4 (CSPG4) receptor and a frizzled protein (FZD) receptor.

In some embodiments, the RDA is able to block a C. difficile toxin from binding either a chondroitin sulfate proteoglycan 4 (CSPG4) receptor or a frizzled protein (FZD) receptor or both.

TABLE 1

| | | SEQ |
|---|---|---|
| | | ID |
| Name: | Sequences | NO: |
| TcdB1 | MSLVNRKQLEKMANVRFRTQEDEYVAILDA | 1 |
| (1-2366)- | LEEYHNMSENTVVEKYLKLKDINSLTDIYI | |
| holotoxin | DTYKKSGRNKALKKFKEYLVTEVLELKNNN | |
| | LTPVEKNLHFVWIGGQINDTAINYINQWKD | |
| | VNSDYNVNVFYDSNAFLINTLKKTVVESAI | |
| | NDTLESFRENLNDPRFDYNKFFRKRMEIIY | |
| | DKQKNFINYYKAQREENPELIIDDIVKTYL | |
| | SNEYSKEIDELNTYIEESLNKITQNSGNDV | |
| | RNFEEFKNGESFNLYEQELVERWNLAAASD | |
| | ILRISALKEIGGMYLDVDMLPGIQPDLFES | |
| | IEKPSSVTVDFWEMTKLEAIMKYKEYIPEY | |
| | TSEHFDMLDEEVQSSFESVLASKSDKSEIF | |
| | SSLGDMEASPLEVKIAFNSKGIINQGLISV | |
| | KDSYCSNLIVKQIENRYKILNNSLNPAISE | |
| | DNDFNTTTNTFIDSIMAEANADNGRFMMEL | |
| | GKYLRVGFFPDVKTTINLSGPEAYAAAYQD | |
| | LLMFKEGSMNIHLIEADLRNFEISKTNISQ | |
| | STEQEMASLWSFDDARAKAQFEEYKRNYFE | |
| | GSLGEDDNLDFSQNIVVDKEYLLEKISSLA | |
| | RSSERGYIHYIVQLQGDKISYEAACNLFAK | |
| | TPYDSVLFQKNIEDSEIAYYYNPGDGEIQE | |
| | IDKYKIPSIISDRPKIKLTFIGHGKDEFNT | |
| | DIFAGFDVDSLSTEIEAAIDLAKEDISPKS | |
| | IEINLLGCNMFSYSINVEETYPGKLLLKVK | |
| | DKISELMPSISQDSIIVSANQYEVRINSEG | |
| | RRELLDHSGEWINKEESIIKDISSKEYISF | |
| | NPKENKITVKSKNLPELSTLLQEIRNNSNS | |
| | SDIELEEKVMLTECEINVISNIDTQIVEER | |
| | IEEAKNLTSDSINYIKDEFKLIESISDALC | |
| | DLKQQNELEDSHFISFEDISETDEGFSIRF | |
| | INKETGESIFVETEKTIFSEYANHITEEIS | |
| | KIKGTIFDTVNGKLVKKVNLDTTHEVNTLN | |
| | AAFFIQSLIEYNSSKESLSNLSVAMKVQVY | |
| | AQLFSTGLNTITDAAKVVELVSTALDETID | |
| | LLPTLSEGLPIIATIIDGVSLGAAIKELSE | |
| | TSDPLLRQEIEAKIGIMAVNLTTATTAIIT | |
| | SSLGIASGFSILLVPLAGISAGIPSLVNNE | |
| | LVLRDKATKVVDYFKHVSLVETEGVFTLLD | |
| | DKIMMPQDDLVISEIDFNNNSIVLGKCEIW | |
| | RMEGGSGHTVTDDIDHFFSAPSITYREPHL | |
| | SIYDVLEVOKEELDLSKDLMVLPNAPNRVF | |
| | AWETGWTPGLRSLENDGTKLLDRIRDNYEG | |
| | EFYWRYFAFIADALITTLKPRYEDTNIRIN | |
| | LDSNTRSFIVPIITTEYIREKLSYSFYGSG | |
| | GTYALSLSQYNMGINIELSESDVWIIDVDN | |
| | VVRDVTIESDKIKKGDLIEGILSTLSIEEN | |
| | KIILNSHEINFSGEVNGSNGFVSLTFSILE | |
| | GINAIIEVDLLSKSYKLLISGELKILMLNS | |
| | NHIQQKIDYIGFNSELQKNIPYSFVDSEGK | |
| | ENGFINGSTKEGLFVSELPDVVLISKVYMD | |
| | DSKPSFGYYSNNLKDVKVITKDNVNILTGY | |
| | YLKDDIKISLSLTLQDEKTIKLNSVHLDES | |
| | GVAEILKFMNRKGNTNTSDSLMSFLESMNI | |
| | KSIFVNFLQSNIKFILDANFIISGTTSIGQ | |
| | FEFICDENDNIQPYFIKFNTLETNYTLYVG | |
| | NRQNMIVEPNYDLDDSGDISSTVINFSQKY | |
| | LYGIDSCVNKVVISPNIYTDEINITPVYET | |
| | NNTYPEVIVLDANYINEKINVNINDLSIRY | |
| | VWSNDGNDFILMSTSEENKVSQVKIRFVNV | |
| | FKDKTLANKLSFNFSDKQDVPVSEIILSFT | |
| | PSYYEDGLIGYDLGLVSLYNEKFYINNFGM | |
| | MVSGLIYINDSLYYFKPPVNNLITGFVTVG | |
| | DDKYYFNPINGGAASIGETIIDDKNYYFNQ | |
| | SGVLQTGVFSTEDGFKYFAPANTLDENLEG | |
| | EAIDFTGKLIIDENIYYFDDNYRGAVEWKE | |
| | LDGEMHYFSPETGKAFKGLNQIGDYKYYFN | |
| | SDGVMQKGFVSINDNKHYFDDSGVMKVGYT | |
| | EIDGKHFYFAENGEMQIGVENTEDGFKYFA | |
| | HHNEDLGNEEGEEISYSGILNFNNKIYYFD | |
| | DSFTAVVGWKDLEDGSKYYFDEDTAEAYIG | |
| | LSLINDGQYYFNDDGIMQVGFVTINDKVFY | |
| | FSDSGIIESGVQNIDDNYFYIDDNGIVQIG | |
| | VFDTSDGYKYFAPANTVNDNIYGQAVEYSG | |
| | LVRVGEDVYYFGETYTIETGWIYDMENESD | |
| | KYYFNPETKKACKGINLIDDIKYYFDEKGI | |
| | MRTGLISFENNNYYFNENGEMQFGYINIED | |
| | KMFYFGEDGVMQIGVFNTPDGFKYFAHQNT | |
| | LDENFEGESINYTGWLDLDEKRYYFTDEYI | |
| | AATGSVIIDGEEYYFDPDTAQLVISE | |
| Repeat1 | ELPEPCVPEPGLPPVFANFTQLLTISPLVV | 2 |
| (410-551) | AEGGTAWLEWRHVQPTLDLMEAELRKSQVL | |
| | FSVTRGARHGELELDIPGAQARKMFTLLDV | |
| | VNRKARFIHDGSEDTSDQLVLEVSVTARVP | |
| | MPSCLRRGQTYLLPIQVNPVND | |
| LG2- | ASFFGENHLEVPVATALTDIDLQLQFSTSQ | 3 |
| Repeat1 | PEALLLLAAGPADHLLLQLYSGRLQVRLVL | |
| (30-551) | GQEELRLQTPAETLLSDSIPHTVVLTVVEG | |
| | WATLSVDGFLNASSAVPGAPLEVPYGLFVG | |
| | GTGTLGLPYLRGTSRPLRGCLHAATLNGRS | |
| | LLRPLTPDVHEGCAEEFSASDDVALGFSGP | |
| | HSLAAFPAWGTQDEGTLEFTLTTQSRQAPL | |
| | AFQAGGRRGDFIYVDIFEGHLRAVVEKGQG | |
| | TVLLHNSVPVADGQPHEVSVHINAHRLEIS | |
| | VDQYPTHTSNRGVLSYLEPRGSLLLGGLDA | |
| | EASRHLQEHRLGLTPEATNASLLGCMEDLS | |
| | VNGQRRGLREALLTRNMAAGCRLEEEEYED | |
| | DAYGHYEAFSTLAPEAWPAMELPEPCVPEP | |
| | GLPPVFANFTQLLTISPLVVAEGGTAWLEW | |
| | RHVQPTLDLMEAELRKSQVLFSVTRGARHG | |
| | ELELDIPGAQARKMFTLLDVVNRKARFIHD | |
| | GSEDTSDQLVLEVSVTARVPMPSCLRRGQT | |
| | YLLPIQVNPVND | |
| CRD1 | QYNGERGISIPDHGYCQPISIPLCTDIAYN | 4 |
| (96-253)- | QTIMPNLLGHTNQEDAGLEVHQFYPLVKVQ | |
| from FZD1 | CSAELKFFLCSMYAPVCTVLEQALPPCRSL | |
| | CERARQGCEALMNKFGFQWPDTLKCEKFPV | |
| | HGAGELCVGQNTSDKGTPTPSLLPEFWTSN | |
| | PQHGGGGY | |
| CRD2 | QFHGEKGISIPDHGFCQPISIPLCTDIAYN | 5 |
| (28-191)- | QTIMPNLLGHTNQEDAGLEVHQFYPLVKVQ | |
| from FZD2 | CSPELRFFLCSMYAPVCTVLEQAIPPCRSI | |
| | CERARQGCEALMNKFGFQWPERLRCEHFPR | |
| | HGAEQICVGQNHSEDGAPALLTTAPPSGLQ | |
| | PGAGGTPGGPGGG | |
| CRD7 | QPYHGEKGISVPDHGFCQPISIPLCTDIAY | 6 |
| (33-173)- | NQTILPNLLGHTNQEDAGLEVHQFYPLVKV | |
| from FZD7 | QCSPELRFFLCSMYAPVCTVLDQAIPPCRS | |
| | LCERARQGCEALMNKFGFQWPERLRCENFP | |
| | VHGAGEICVGQNTSDGSGGAG | |
| CSPG4 | MQSGPRPPLPAPGLALALTLTMLARLASAA | 7 |
| (1-2322)- | SFFGENHLEVPVATALTDIDLQLQFSTSQP | |
| full | EALLLLAAGPADHLLLQLYSGRLQVRLVLG | |
| length | QEELRLQTPAETLLSDSIPHTVVLTVVEGW | |
| | ATLSVDGFLNASSAVPGAPLEVPYGLFVGG | |
| | TGTLGLPYLRGTSRPLRGCLHAATLNGRSL | |
| | LRPLTPDVHEGCAEEFSASDDVALGFSGPH | |
| | SLAAFPAWGTQDEGTLEFTLTTQSRQAPLA | |
| | FQAGGRRGDFIYVDIFEGHLRAVVEKGQGT | |
| | VLLHNSVPVADGQPHEVSVHINAHRLEISV | |
| | DQYPTHTSNRGVLSYLEPRGSLLLGGLDAE | |
| | ASRHLQEHRLGLTPEATNASLLGCMEDLSV | |
| | NGQRRGLREALLTRNMAAGCRLEEEEYEDD | |
| | AYGHYEAFSTLAPEAWPAMELPEPCVPEPG | |
| | LPPVFANFTQLLTISPLVVAEGGTAWLEWR | |
| | HVQPTLDLMEAELRKSQVLFSVTRGARHGE | |
| | LELDIPGAQARKMFTLLDVVNRKARFIHDG | |
| | SEDTSDQLVLEVSVTARVPMPSCLRRGQTY | |
| | LLPIQVNPVNDPPHIIFPHGSLMVILEHTQ | |
| | KPLGPEVFQAYDPDSACEGLTFQVLGTSSG | |
| | LPVERRDQPGEPATEFSCRELEAGSLVYVH | |
| | RGGPAQDLTFRVSDGLQASPPATLKVVAIR | |
| | PAIQIHRSTGLRLAQGSAMPILPANLSVET | |
| | NAVGQDVSVLFRVTGALQFGELQKQGAGGV | |
| | EGAEWWATQAFHQRDVEQGRVRYLSTDPQH | |
| | HAYDTVENLALEVQVGQEILSNLSFPVTIQ | |
| | RATVWMLRLEPLHTQNTQQETLTTAHLEAT | |
| | LEEAGPSPPTFHYEVVQAPRKGNLQLQGTR | |
| | LSDGQGFTQDDIQAGRVTYGATARASEAVE | |
| | DTFRFRVTAPPYFSPLYTFPIHIGGDPDAP | |
| | VLTNVLLVVPEGGEGVLSADHLFVKSLNSA | |
| | SYLYEVMERPRHGRLAWRGTQDKTTMVTSF | |
| | TNEDLLRGRLVYQHDDSETTEDDIPFVATR | |
| | QGESSGDMAWEEVRGVFRVAIQPVNDHAPV | |
| | QTISRIFHVARGGRRLLTTDDVAFSDADSG | |
| | FADAQLVLTRKDLLFGSIVAVDEPTRPIYR | |
| | FTQEDLRKRRVLFVHSGADRGWIQLQVSDG | |
| | QHQATALLEVQASEPYLRVANGSSLVVPQG | |
| | GQGTIDTAVLHLDTNLDIRSGDEVHYHVTA | |
| | GPRWGQLVRAGQPATAFSQQDLLDGAVLYS | |
| | HNGSLSPRDTMAFSVEAGPVHTDATLQVTI | |
| | ALEGPLAPLKLVRHKKIYVFQGEAAEIRRD | |
| | QLEAAQEAVPPADIVFSVKSPPSAGYLVMV | |
| | SRGALADEPPSLDPVQSFSQEAVDTGRVLY | |
| | LHSRPEAWSDAFSLDVASGLGAPLEGVLVE | |
| | LEVLPAAIPLEAQNFSVPEGGSLTLAPPLL | |
| | RVSGPYFPTLLGLSLQVLEPPQHGALQKED | |
| | GPQARTLSAFSWRMVEEQLIRYVHDGSETL | |
| | TDSFVLMANASEMDRQSHPVAFTVTVLPVN | |
| | DQPPILTTNTGLQMWEGATAPIPAEALRST | |
| | DGDSGSEDLVYTIEQPSNGRVVLRGAPGTE | |
| | VRSFTQAQLDGGLVLFSHRGTLDGGFRFRL | |
| | SDGEHTSPGHFFRVTAQKQVLLSLKGSQTL | |
| | TVCPGSVQPLSSQTLRASSSAGTDPQLLLY | |
| | RVVRGPQLGRLFHAQQDSTGEALVNFTQAE | |
| | VYAGNILYEHEMPPEPFWEAHDTLELQLSS | |
| | PPARDVAATLAVAVSFEAACPQRPSHLWKN | |
| | KGLWVPEGQRARITVAALDASNLLASVPSP | |
| | QRSEHDVLFQVTQFPSRGQLLVSEEPLHAG | |
| | QPHFLQSQLAAGQLVYAHGGGGTQQDGFHF | |
| | RAHLQGPAGASVAGPQTSEAFAITVRDVNE | |
| | RPPQPQASVPLRLTRGSRAPISRAQLSVVD | |
| | PDSAPGEIEYEVQRAPHNGFLSLVGGGLGP | |
| | VTRFTQADVDSGRLAFVANGSSVAGIFQLS | |
| | MSDGASPPLPMSLAVDILPSAIEVQLRAPL | |
| | EVPQALGRSSLSQQQLRVVSDREEPEAAYR | |
| | LIQGPQYGHLLVGGRPTSAFSQFQIDQGEV | |
| | VFAFTNFSSSHDHFRVLALARGVNASAVVN | |
| | VTVRALLHVWAGGPWPQGATLRLDPTVLDA | |
| | GELANRTGSVPRFRLLEGPRHGRVVRVPRA | |
| | RTEPGGSQLVEQFTQQDLEDGRLGLEVGRP | |
| | EGRAPGPAGDSLTLELWAQGVPPAVASLDF | |
| | ATEPYNAARPYSVALLSVPEAARTEAGKPE | |
| | SSTPTGEPGPMASSPEPAVAKGGFLSFLEA | |
| | NMFSVIIPMCLVLLLLALILPLLFYLRKRN | |
| | KTGKHDVQVLTAKPRNGLAGDTETFRKVEP | |
| | GQAIPLTAVPGQGPPPGGQPDPELLQFCRT | |
| | PNPALKNGQYWV | |
| VHH | QVQLVESGGGLVQPGGSLRLSCAASGFTLD | 8 |
| nanobody | YYGIGWFRQAPGQGLEAVSYISASARTILY | |
| (humanized | ADSVKGRFTISRDNSKNTLYLQMNSLRAED | |
| 5D) | TAVYYCARRRFSASSVNRWLADDYDVWGQG | |
| | TLVTVSS | |

In some embodiments, the frizzled protein (FZD) receptor portion of the RDA composition comprises a peptide that is at least 70% identical to a frizzled (FZD) protein or a fragment thereof. In some embodiments, the FZD portion of the RDA composition comprises a peptide that is at least 75% identical to an FZD protein or a fragment thereof. In some embodiments, the FZD portion of the RDA composition comprises a peptide that is at least 80% identical to an FZD protein or a fragment thereof. In some embodiments, the FZD portion of the RDA composition comprises a peptide that is at least 85% identical to an FZD protein or a fragment thereof. In some embodiments, the FZD portion of the RDA composition comprises a peptide that is at least 90% identical to an FZD protein or a fragment thereof. In some embodiments, the FZD portion of the RDA composition comprises a peptide that is at least 95% identical to an FZD protein or a fragment thereof. In some embodiments, the FZD portion of the RDA composition comprises a peptide that is at least 99% identical to an FZD protein or a fragment thereof. In some embodiments, the FZD portion of the RDA composition comprises a peptide that is at least 100% identical to an FZD protein or a fragment thereof.

In some embodiments, the fragment of the frizzle protein (FZD) receptor comprises a cysteine rich domain of a FZD protein. In some embodiments, the cysteine rich domain (CRD) portion of the RDA composition comprises a peptide that is at least 70% identical to a frizzled (FZD) protein or a fragment thereof. In some embodiments, the CRD portion of the RDA composition comprises a peptide that is at least 75% identical to an FZD protein or a fragment thereof. In some embodiments, the CRD portion of the RDA composition comprises a peptide that is at least 80% identical to an FZD protein or a fragment thereof. In some embodiments, the CRD portion of the RDA composition comprises a peptide that is at least 85% identical to an FZD protein or a fragment thereof. In some embodiments, the CRD portion of the RDA composition comprises a peptide that is at least 90% identical to an FZD protein or a fragment thereof. In some embodiments, the CRD portion of the RDA composition comprises a peptide that is at least 95% identical to an FZD protein or a fragment thereof. In some embodiments, the CRD portion of the RDA composition comprises a peptide that is at least 99% identical to an FZD protein or a fragment thereof. In some embodiments, the CRD portion of the RDA composition comprises a peptide that is at least 100% identical to an FZD protein or a fragment thereof.

In some embodiments, the cysteine rich domain (CRD) may be from a FZD1 protein, or an FZD2 protein, or an FZD7 protein. In some embodiments the CRD portion of the RDA may be mutated. In some embodiments, the mutation of the CRD portion makes the RDA unable to bind to WNT proteins, but still able to bind to the TcdB toxin. In some embodiments, the CRD portion may be comprised of SEQ ID NO: 4, SEQ ID NO: 5, or SEQ ID NO: 6. The CRD portion is not limited to the sequences described herein.

In some embodiments, the chondroitin sulfate proteoglycan 4 (CSPG4) rniniicking fragment (the decoy) is a CSPG4 fragment that includes residues 30-551 (SEQ ID NO: 3). In some embodiments, the CSPG4 fragment is sufficient to bind to TcdB. In some embodiments, the core of the CSPG4 decoy is composed of residues 410-551 (termed Repeat1—SEQ ID NO: 2), which is minimally required to bind TcdB.

In some embodiments, the CSPG4 fragment is about 10 to 25 amino acids (aa) in length. In some embodiments, the CSPG4 fragment is about 10 to 50 aa in length. In some embodiments, the CSPG4 fragment is about 10 to 100 aa in length. In some embodiments, the CSPG4 fragment is about 10 to 150 aa in length. In some embodiments, the CSPG4 fragment is about 10 to 200 aa in length. In some embodiments, the CSPG4 fragment is about 10 to 250 aa in length. In some embodiments, the CSPG4 fragment is about 10 to 300 aa in length. In some embodiments, the CSPG4 fragment is about 10 to 350 aa in length. In some embodiments, the CSPG4 fragment is about 10 to 400 aa in length. In some embodiments, the CSPG4 fragment is about 10 to 450 aa in length. In some embodiments, the CSPG4 fragment is about 10 to 500 aa in length. In some embodiments, the CSPG4 fragment is about 10 to 550 aa in length. In some embodiments, the CSPG4 fragment is about 25 to 50 aa in length. In some embodiments, the CSPG4 fragment is about 25 to 100 aa in length. In some embodiments, the CSPG4 fragment is about 25 to 150 aa in length. In some embodiments, the CSPG4 fragment is about 25 to 200 aa in length. In some embodiments, the CSPG4 fragment is about 25 to 250 aa in length. In some embodiments, the CSPG4 fragment is about 25 to 300 aa in length. In some embodiments, the CSPG4 fragment is about 25 to 350 aa in length. In some embodiments, the CSPG4 fragment is about 25 to 400 aa in length. In some embodiments, the CSPG4 fragment is about 25 to 450 aa in length. In some embodiments, the CSPG4 fragment is about 25 to 500 aa in length. In some embodiments. the CSPG4 fragment is about 25 to 550 aa in length. In some embodiments, the CSPG4 fragment is about 50 to 100 aa in length. In some embodiments, the CSPG4 fragment is about 50 to 150 aa in length. In some embodiments, the CSPG4 fragment is about 50 to 200 aa in length. In some embodiments, the CSPG4 fragment is about 50 to 250 aa in length. In some embodiments, the CSPG4 fragment is about 50 to 300 aa in length. In some embodiments, the CSPG4 fragment is about 50 to 350 aa in length. In some embodiments, the CSPG4 fragment is about 50 to 400 aa in length. In some embodiments, the CSPG4 fragment is about 50 to 450 aa in length. In some embodiments, the CSPG4 fragment is about 50 to 500 aa in length. In some embodiments, the CSPG4 fragment is about 50 to 550 aa in length. In some embodiments, the CSPG4 fragment is about 100 to 150 aa in length. In some embodiments, the CSPG4 fragment is about 100 to 200 aa in length. In some embodiments, the CSPG4 fragment is about 100 to 250 aa in length. In some embodiments, the CSPG4 fragment is about 100 to 300 aa in length. In some embodiments, the CSPG4 fragment is about 100 to 350 aa in length. In some embodiments, the CSPG4 fragment is about 100 to 400 aa in length. In some embodiments, the CSPG4 fragment is about 100 to 450 aa in length. In some embodiments, the CSPG4 fragment is about 100 to 500 aa in length, In some embodiments, the CSPG4 fragment is about 100 to 550 aa in length. In some embodiments, the CSPG4 fragment is about 150 to 200 aa in length. In some embodiments, the CSPG4 fragment is about 150 to 250 aa in length. In some embodiments, the CSPG4 fragment is about 150 to 300 aa in length. In some embodiments, the CSPG4 fragment is about 150 to 350 aa in length. In some embodiments, the CSPG4 fragment is about 150 to 400 aa in length. In some embodiments, the CSPG4 fragment is about 150 to 450 aa in length. In some embodiments, the CSPG4 fragment is about 150 to 500 aa in length. In some embodiments, the CSPG4 fragment is about 150 to 550 aa in length. In some embodiments, the CSPG4 fragment is about 200 to 250 aa in length. In some embodiments, the CSPG4 fragment is about 200 to 300 aa in length. In some embodiments, the CSPG4 fragment is about 200 to 350 aa in length. In some embodiments, the CSPG4 fragment is about 250 to 300 aa in length. In some embodiments, the CSPG4 fragment is about 250 to 350 aa in length. In some embodiments, the CSPG4 fragment is about 250 to 400 aa in length. In some embodiments, the CSPG4 fragment is about 250 to 450 aa in length. In some embodiments, the CSPG4 fragment is about 250 to 400 aa in length. In some embodiments, the CSPG4 fragment is about 250 to 500 aa in length. In some embodiments, the CSPG4 fragment is about 250 to 550 aa in length. In some embodiments, the CSPG4 fragment is more than 550 aa in length.

In some embodiments, the CSPG4 portion of the RDA composition comprises a peptide that is at least 80% identical to the CSPG4 protein or a fragment thereof. In some embodiments, the CSPG4 portion of the RDA composition comprises a peptide that is at least 85% identical to an CSPG4 protein or a fragment thereof. In some embodiments, the CSPG4 portion of the RDA composition comprises a peptide that is at least 90% identical to an CSPG4 protein or a fragment thereof. In some embodiments, the CSPG4 portion of the RDA composition comprises a peptide that is at least 95% identical to an CSPG4 protein or a fragment thereof. In some embodiments, the CSPG4 portion of the RDA composition comprises a peptide that is at least 99% identical to an CSPG4 protein or a fragment thereof. In some embodiments, the CSPG4 portion of the RDA composition comprises a peptide that is at least 100% identical to an CSPG4 protein or a fragment thereof.

In some embodiments, the CSPG4 fragment is recombinantly produced and purified. In some embodiments, the CSPG4 fragment is highly expressed.

As used herein “the fragment crystallizable region, or the fragment constant region or Fc region or Fc” may be used interchangeably and refer to the tail region of an antibody that interacts with cell surface receptors. In some embodiments, the Fc region may include, but is not limited to the Fc region of IgG1, IgG2, IgG3, IgG4, IgA, IgD, IgE or IgM. In some embodiments, the Fc region would confer the stability, distribution, and half-life similar to the Ig protein used to create the Fc region. In some embodiments, the Fc region is modified to regulate its interaction with Fc receptors (abbreviated FcR).

In some embodiments, the Fc region may be mutated. In some embodiments, a mutation in the Fc region may cause the pharmacokinetics (PK) to be prolonged. In some embodiments, a mutation in the Fc region may modulate the antibody-dependent cellular cytotoxicity (ADCC). In some embodiments, a mutation in the Fc region may increase the ADCC. In some embodiments, a mutation in the Fc region may decrease the ADCC.

In some embodiments, the Fc portion of the RDA composition comprises a peptide that is at least 80% identical to an Fc region or a fragment thereof. In some embodiments, the Fc portion of the RDA composition comprises a peptide that is at least 85% identical to an Fc protein or a fragment thereof. In some embodiments, the Fc portion of the RDA composition comprises a peptide that is at least 90% identical to an Fc protein or a fragment thereof. In some embodiments, the Fc portion of the RDA composition comprises a peptide that is at least 95% identical to an Fc protein or a fragment thereof. In some embodiments, the Fc portion of the RDA composition comprises a peptide that is at least 99% identical to an Fc protein or a fragment thereof. In some embodiments, the Fc portion of the RDA composition comprises a peptide that is at least 100% identical to an Fc protein or a fragment thereof.

As used herein, the “VHH nanobody,” “VHH 5D nanobody,” or the 5D nanobody may be used interchangeably and refers to the antigen binding fragment of heavy chain only antibodies. In some embodiments, the VHH nanobody is a 5D nanobody. In other embodiments, the VHH 5D nanobody is a humanized VHH 5D nanobody (SEQ ID NO: 8). In some embodiments, a humanized VHH 5D nanobody has low or no immunogenicity compared to the WT 5D. In some embodiments, a full length VHH 5D nanobody is incorporated into the RDA composition as described herein. In other embodiments, a fragment of the VHH 5D nanobody is incorporated into the RDA composition as described herein.

In some embodiments, the 5D nanobody portion of the RDA composition comprises a peptide that is at least 80% identical to an humanized 5D nanobody or a fragment thereof. In some embodiments, the 5D nanobody portion of the RDA composition comprises a peptide that is at least 85% identical to an humanized 5D nanobody or a fragment thereof. In some embodiments, the 5D nanobody portion of the RDA composition comprises a peptide that is at least 90% identical to an humanized 5D nanobody or a fragment thereof. In some embodiments, the 5D nanobody portion of the RDA composition comprises a peptide that is at least 95% identical to an humanized 5D nanobody or a fragment thereof. In some embodiments, the 5D nanobody portion of the RDA composition comprises a peptide that is at least 98% identical to an humanized 5D nanobody or a fragment thereof. In some embodiments, the 5D nanobody portion of the RDA composition comprises a peptide that is at least 99% identical to an humanized 5D nanobody or a fragment thereof. In some embodiments, the 5D nanobody portion of the RDA composition comprises a peptide that is at least 100% identical to an humanized 5D nanobody or a fragment thereof

In some embodiments, a peptide linker is used to connect CSPG4 and CRD or CSPG4/CRD to the Fc region. In other embodiments, a peptide linker is used to connect CSPG4 and VHH or CSPG4/VHH to the Fc region. In further embodiments, a peptide linker is used to connect CRD and VHH or CRD/VHH to the Fc region. In some embodiments, the peptide linker length may be adjusted in order to achieve a favorable separation between CSPG4/CRD and Fc and improve the bioactivity of the fusion protein. In some embodiments, the peptide linker may be 0-35 amino acids in length or longer.

In some embodiments, the present invention may also feature a method of neutralizing a toxin of C. difficile. In some embodiments, the method comprises producing a neutralizing receptor decoy antibody (RDA) composition as described herein that binds to C. difficile toxin and blocks it from binding to cell surface receptors. In other embodiments, the present invention features a method of neutralizing a toxin of C. difficile. In some embodiments, the method comprises producing a neutralizing receptor decoy antibody (RDA) composition that binds to C. difficile toxin and blocks it from binding to cell surface receptors. In some embodiments, the RDA composition comprises a fusion protein comprising a Fc region fragment, and a fragment of a chondroitin sulfate proteoglycan 4 (CSPG4) receptor tandernly attached to the Fc region.

In some embodiments, the RDA binds to the TcdB1 toxin. In other embodiments, the RDA binds to the TcdB2 toxin. Other non-limiting examples of TcdB subtypes the RDA can bind to include but are not limited to TcdB3, TcdB4, TcdB5, TcdB6, TcdB7, TcdB8, TcdB9, TcdB10, TcdB11, or TcdB12 (see FIG. 18A-18D).

In some embodiments, the RDA mimics a chondroitin sulfate proteoglycan 4 (CSPG4) receptor. In some embodiments, the RDA mimics a frizzled protein (FZD) receptor. In other embodiments, the RDA mimics both a chondroitin sulfate proteoglycan 4 (CSPG4) receptor and frizzled protein (FZD) receptor. Additionally, in some embodiments, the RDA is able to block C. difficile from binding either a chondroitin sulfate proteoglycan 4 (CSPG4) receptor or a frizzled protein (FZD) receptor or both.

Additionally, in some embodiments, the present invention may feature a method of treating a Clostridium difficile infection (CDD) in a patient in need thereof. In some embodiments, the method comprises administering a standard of care (SOC) antibiotic and administering a therapeutically effective dose of a neutralizing receptor decoy antibody (RDA) composition as described herein.

In some embodiments of the present invention, the RDA may be administered in a dosage of about 0.1 mg/kg body weight to 50 mg/kg body weight. For example, the dosage may range from about 0.1 mg/kg body weight to 0.5 mg/kg body weight, or about 0.5 mg/kg body weight to 1 mg/kg body weight, or about 1 mg/kg body weight to 2 mg/kg body weight, or about 2 mg/kg body weight to 3 mg/kg body weight, or about 3 mg/kg body weight to 4 mg/kg body weight, or about 4 mg/kg body weight to 5 mg/kg body weight, or about 5 mg/kg body weight to 6 mg/kg body weight, or about 6 mg/kg body weight to 7 mg/kg body weight, or about 7 mg/kg body weight to 8 mg/kg body weight, or about 8 mg/kg body weight to 9 mg/kg body weight, or about 9 mg/kg body weight to 10 mg/kg body weight, or about 10 mg/kg body weight to 11 mg/kg body weight, or about 11 mg/kg body weight to 12 mg/kg body weight or about 12 mg/kg body weight to 13 mg/kg body weight, or about 13 mg/kg body weight to 14 mg/kg body weight, or about 14 mg/kg body weight to 15 mg/kg body weight, or about 15 mg/kg body weight to 16 mg/kg body weight, or about 16 mg/kg body weight to 17 mg/kg body weight, or about 17 mg/kg body weight to 18 mg/kg body weight, or about 18 mg/kg body weight to 19 mg/kg body weight, or about 19 mg/kg body weight to 20 mg/kg body weight, or about 20 mg/kg body weight to 25 mg/kg body weight, or about 25 mg/kg body weight to 30 mg/kg body weight, or about 30 mg/kg body weight to 35 mg/kg body weight, or about 35 mg/kg body weight to 40 mg/kg body weight, or about 40 mg/kg body weight to 45 mg/kg body weight, or about 45 mg/kg body weight to 50 mg/kg body weight.

In some embodiments of the present invention, the RDA may be administered in a dosage of about 0.1 mg/kg to 50 mg/kg For example, the dosage may range from about 0.1 mg/kg to 1 mg/kg, or about 1 mg/kg to 5 mg/kg, or about 5 mg/kg to 10 mg/kg, or about 10 mg/kg to 15 mg/kg, or about 15 mg/kg to 20 mg/kg, or about 20 mg/kg to 25 mg/kg, or about 25 mg/kg to 30 mg/kg, or about 30 mg/kg to 35 mg/kg, or about 35 mg/kg to 40 mg/kg, or about 40 mg/kg to 45 mg/kg, or about 45 mg/kg to 50 mg/kg.

In some embodiments, the RDA composition described herein for use may be administered once daily or twice daily. In another embodiment, the RDA composition described herein may be administered at least once to four times daily. In some embodiment, the RDA composition described herein may be administered at least once daily, at least once every other day, or at least once weekly or at least bi-weekly, or at least monthly. In another embodiment, the RDA composition described herein may be administered continuously by an intravenous drip. In other embodiments, the RDA composition described herein may be administered orally. In other embodiments, the RDA composition described herein is administered at a daily dose ranging from about 0.1 mg/kg of body weight to 50 mg/kg of body weight. In some embodiments, the RDA composition described herein is administered at a weekly dose ranging from about 0.1 mg/kg of body weight to 50 mg/kg of body weight. In some embodiments, the RDA is administered at a bi-weekly dose ranging from about 0.1 mg/kg of body weight to 50 mg/kg of body weight. In some embodiments, the RDA is administered at a monthly dose of about 0.1 mg/kg of body weight to 50 mg/kg of body weight. Further still, the RDA composition described herein may be administered intravenously. In preferred embodiments, the RDA for use in the treatment resulted in clinical improvement of CDI caused by Clostridium difficile toxins.

In some embodiments, the neutralizing receptor decay antibody (RDA) composition can be used as a standalone treatment. In some embodiments, the RDA composition is used along with the standard-of-care (SOC) COI antibiotic administration. In some embodiments, SOC CDI antibiotics may include, but are not limited to vancomycin, fidaxomicin, metronidazole or bezlotoxurnab. In some embodiments, the SOC CDI antibiotics are given orally. In some embodiments, the RDA can be used with fecal microbiota transplant. In some embodiments, the RDA composition may be used with oral microbiome therapy.

In other embodiments, the neutralizing receptor decoy antibody (RDA) can be given to healthy patients, who do not have CDI. In some embodiments, the neutralizing receptor decoy antibody (RDA) can be given to prevent CDI in a subject. In further embodiments, the neutralizing receptor decoy antibody (RDA) can be given prophylactically to a subject. In some embodiments, the RDA can be given to patients who are receiving antibacterial drug treatment for other diseases. In other embodiments, the RDA is given to patients who are receiving antibacterial drug treatment for other diseases, to reduce CDI symptoms if the patients are infected with C. difficile. In some embodiments, the RDA can be given to cancer patients. In some embodiments, the RDA can be given to cancer patients, to reduce CDI symptoms if the cancer patients are infected with C. difficile.

In some embodiments, the RDA is a bi-specific RDA composition. In other embodiments the RDA is a mono-specific RDA composition. In further embodiments, the RDA is a tri-specific RDA composition.

Additionally, the present invention features a method of treating and/or preventing a Clostridium difficile infection (CDI) with a vaccine composed of the chondroitin sulfate proteoglycan 4 (CSPG4)-binding epitope on TcdB in a patient in need thereof. In some embodiments, the method comprises the steps of administering a CSPG4-binding epitope to a patient and elicitind an immune response. In some embodiments, the antibodies produced by the immune response bind to TcdB and prevent it from binding CSPG4 for cell entry and thus provide protection to the patient.

Finally, the present invention features a method of diagnosing a Clostridium difficile infection (CDI) with a neutralizing reception decoy antibody (RDA) in a patient in need thereof. In some embodiments, the method comprises obtaining a biological sample from the patient. In other embodiments, the method comprises performing a detection assay on the sample obtained from the patient. In some embodiments, the RAS toxin in a sample is detected by the RDA. In some embodiments, the detection of TcdB toxin in a patient's sample is indicative of CDI.

In some embodiments, the RDA as described herein binds to highly conserved regions for TcdB toxin variant (see FIG. 18A-18D). In some embodiments, the RDA as described herein have sub-picomolar affinity against a TcdB toxin (e.g., high affinity). Without wishing to limit the present invention to any theory or mechanism it is believed that the high affinity and broad specificity of the RDA will allow the RDA to capture and enrich most if not all variants of RAS toxins from a patent, which is usually at extremely low concentrations.

In some embodiments, the TcdB toxin may be detected using an RDA as described herein to label the TcdB toxin, once labeled with the RDA a second reagent (e.g., an anti-Fc antibody) may be used to detect the RDA. In other embodiments, the TcdB toxin may be detected using an RDA as described herein to enrich and/or concentrate the TcdB toxin from a patient sample, and then use a second second reagent (e.g. an anti-TcdB antibody) to directly detect TcdB. In further embodiments, the present invention is not limited to any particular method of using an RDA as described herein to detect a TcdB toxin.

In some embodiments, the biological sample obtained from a patient is a blood sample. In other embodiments, the biological sample obtained from a patient is a stool sample. In some embodiments, the soluble components are extracted from the stool sample. In some embodiments, biological samples obtained from a patient are processed accordingly based on the detection assay that will be used on the sample.

In some embodiments, the detection assay is an enzyme immunoassay (EIA). In some embodiments, the detection assay is an enzyme linked immunosorbent assay (ELISA). In some embodiments, the detection assay is a colloidal gold immunochromatographic assay (GICA). The present invention is not limited to the detection assays listed herein, in some embodiments, the detection assay can be any assay similar to the assays described herein.

In some embodiments, the biological samples may include but are not limited to stool, serum, or gastrointestinal tissue samples. In other embodiments, the biological samples may include any tissue samples removed from the gastrointestinal tract (GI) of a patient by a doctor during a medical procedure.

In some embodiments, the present invention features a composition comprising a fusion protein comprising a fragment of a Fc region; and a fragment of a chondroitin sulfate proteoglycan 4 (CSPG4) receptor tandemly attached to the Fc region for use in a method for the treatment of Clostridium difficile infection (CDI). In other embodiments, the present invention features a composition comprising a fusion protein comprising a fragment of a Fc region; and a fragment of a chondroitin sulfate proteoglycan 4 (CSPG4) receptor tandemly attached to the Fc region for use in a method for the treatment of Clostridium difficile infection (CDI), wherein the composition neutralizes a toxin of C. difficile.

In some embodiments, the present invention features a composition comprising a neutralizing receptor decoy antibody (RDA), wherein the RDA comprises a fusion protein comprising a fragment of a Fc region; and a fragment of a chondroitin sulfate proteoglycan 4 (CSPG4) receptor tandemly attached to the Fc region for use in a method for the treatment of Clostridium difficile infection (CDI). In other embodiments, the present invention features a composition comprising a neutralizing receptor decoy antibody (RDA), wherein the RDA comprises a fusion protein comprising a fragment of a Fc region; and a fragment of a chondroitin sulfate proteoglycan 4 (CSPG4) receptor tandemly attached to the Fc region for use in a method for the treatment of Clostridium difficile infection (CDI), wherein the composition neutralizes a toxin of C. difficile.

### EXAMPLE 1

The following is a non-limiting example of the present invention. It is to be understood that said example is not intended to limit the present invention in any way. Equivalents or substitutes are within the scope of the present invention.

Cloning, expression, and purification of recombinant proteins. The genes of TcdB^{core} (residues 1-1967 of VPI10463 strain) and the full-length wild-type TcdB1 were cloned into modified pET22b and pET28a vectors, respectively, with a Twin-Strep tag followed by a human rhinovirus 3C protease cleavage site introduced to its N-terminus and a 6xHis tag to its C-terminus. Four point mutations (W102A/D286N/D288N/L543A) were introduced to the glucosyltransferase domain (GTD) of TcdB^{core} to eliminate the glucosyltransferase activity and thus its toxicity, which was required by the biosafety regulation at Pacific Northwest Center for Cryo-EM (PNCC). The gene of CSPG4^{mini} (residues 30-764) was cloned into a modified pcDNA vector with a human IL2 signal sequence (MYRMQLLSCIALSLALVTNS; SEQ ID NO: 9), a 9xHis tag, and a factor Xa-cleavage site added to its N-terminus. The gene of CSPG4 Repeat1 (residues 410-551) was cloned into a modified pcDNA vector with a human IgGk signal sequence (METDTLLLVVVLLLWVPGSTG; SEQ ID NO: 10), an 8xHis tag, and a factor Xa-cleavage site added to its N-terminus, and a human Fc tag added to the C-terminus (Repeat1-Fc). The synthesized gene of the light chain of bezlotoxumab (Genewiz) and a His-tagged version of Repeat1 were cloned into the same vector with an 8xHis tag and a factor Xa-cleavage site added to its N-terminus. CSPG4 extracellular domain (residues 30-2204, referred to as CSPG4^{ECD}) was cloned to the same vector with a C-terminal 7xHis tag. The synthesized genes of the complete heavy chain of bezlotoxurnab and its V_{H}-C_{H}1 fragment (Genewiz) were cloned into the same vector, respectively, without any tag. Primers are listed in Table 2. All TcdB and CSPG4 mutants were generated by two-step PCR and verified by DNA sequencing.

TABLE 2
List of primers.

| | | SEQ | | SEQ |
|---|---|---|---|---|
| | Forward primer | ID | Reverse primer | ID |
| | (5′->3′) | NO: | (5′->3′) | NO: |
| TcdB^{core} | GGGAATTCCATATGT | 11 | CCGCTCGAGTTTGAA | 12 |
| | CTGCTTGGTCTCACC | | AGCCTTGCCGGTTTC | |
| | CACAATTCG | | CGGGC | |
| Full | CATGCCATGGGCTCT | 13 | CCGCTCGAGTTAGTG | 14 |
| length | GCTTGGTCTCACCCA | | ATGATGATGATGATG | |
| TcdB1 | CAATTCG | | TTCAG | |
| CSPG4^{mini} | CGCGGATCCGCTTCC | 15 | TTCTTGGACCGGTTA | 16 |
| | TTCTTCGGTGAGAAC | | CTGCACCTCCAGGGC | |
| | CACC | | CAGGTTCTCC | |
| Repeat1- | CGCGGATCCGAGCTG | 17 | CTAGTCTAGAGTCAT | 18 |
| Fc | CCTGAGCCATGCGTG | | TGACAGGGTTGACCT | |
| | CCTG | | GGATG | |
| CSPG4^{ECD} | CCGGAATTCGCTTCC | 19 | CTAGTCTAGAGCTGG | 20 |
| | TTCTTCGGTGAGAAC | | ATGCCATGGGGCCTG | |
| | CACC | | GCTCG | |
| His- | CGCGGATCCGAGCTG | 21 | CTAGTCTAGACTAGT | 22 |
| Repeat1 | CCTGAGCCATGCGTG | | CATTGACAGGGTTGA | |
| | CCTG | | CCTGGATG | |
| Bezlo | AAAAGGCCTGAGATT | 23 | CTAGTCTAGATTAGC | 24 |
| LC | GTGCTGACACAGTCC | | ATTCTCCTCTATTGA | |
| | CCCG | | AGCTCTTGGTC | |
| Bezlo | AAAAGGCCTGAGGTG | 25 | CTAGTCTAGAGCAGG | 26 |
| HC | CAGCTCGTGCAGAGC | | ACTTGGGCTCCACCC | |
| | GGCG | | TCTTATCG | |
| Bezlo | AAAAGGCCTGAGGTG | 27 | CTAGTCTAGATTAGC | 28 |
| V_{H}-C_{H}1 | CAGCTCGTGCAGAGC | | AGGACTTGGGCTCCA | |
| | GGCG | | CCCTCTTATCG | |

TcdB^{core}, the Twin-Strep tagged full-length TcdB1, and all TcdB1 mutants were expressed in E. coli strain BL21-Star (DE3) (Invitrogen). Bacteria were cultured at 37° C. in LB medium containing kanamycin or ampicillin. The temperature was reduced to 18° C. when OD600 reached ˜0.8. Expression was induced with 1 mM IPTG (isopropyl-b-D-thiogalactopyranoside) and continued at 18° C. overnight. The cells were harvested by centrifugation and stored at −80° C. until use. The recombinant full-length TcdB1 (VPI10463 strain) and TcdB2 (R20292 strain), which were used for affinity measurement and competition assays, were expressed in Bacillus megaterium and purified.

The His-tagged proteins (TcdB^{core}, Twin-Strep tagged full-length TcdB1, and TcdB1 mutants) were purified using Ni²⁺-NTA (nitrilotriacetic acid, Qiagen) affinity resins in a buffer comprising 50 mM Tris, pH 8.0, 400 mM NaCl, and 40 mM imidazole. The proteins were eluted with a high-imidazole buffer (50 mM Tris, pH 8.0, 400 mM NaCl, and 300 rnM imidazole) and then dialyzed at 4° C. against a buffer comprising 20 mM HEPES, pH 7.5, and 150 rnM NaCl. The Twin-Strep tagged TcdBcore, TcdB1, and its variants were further purified using Strep-Tactin resins (IBA Lifesciences).

The His-tagged CSPG4^{mini}, CSPG4^{ECD}, Repeat1, Repeat1-Fc and its mutants were expressed and secreted from FreeStyle HEK 293 cells (ThermoFisher) by polyethylenimine (PEI)-mediated transient transfection. Proteins were purified directly from cell culture medium using Ni²⁺-NTA resins, which were then eluted with a buffer comprising 50 mM Tris, pH 8.0, 400 mM NaCl, 3 mM CaCl₂, and 300 mM imidazole. Bezlotoxumab and its Fab were expressed by co-transfection of the light chain and the heavy chain, and the secreted proteins were purified via the His-tag on the light chain using Ni²⁺-NTA resins and the aforementioned buffer. CSPG4^{mini} was further purified by Superdex-200 size-exclusion chromatography using a buffer containing 20 mM HEPES, pH 7.5, 3 mM CaCl₂, and 150 mM NaCl. To prepare the TcdB^{core}-CSPG4^{mini} complex, the purified TcdB^{core} was first bound to Strep-Tactin resins for 3-4 hours and the unbound TcdB^{core} was washed away using a buffer containing 20 mM HEPES, pH 7.5, 3 mM CaCl₂, and 150 mM NaCl. The TcdB-bound resins were then mixed with a 4-fold molar excess of the purified CSPG4^{mini} for 3-4 hours. After the unbound CSPG4^{mini} was washed away, the protein complex was eluted by a buffer comprising 20 mM HEPES, pH 7.5, 3 mM CaCl₂, 50 mM D-biotin, and 150 mM NaCl and then dialyzed at 4° C. against a buffer comprsing 20 mM HEPES, pH 7.5, 3 mM CaCl₂, and 150 mM NaCl. The TcdB-CSPG4^{ECD} complex was assembled using a similar strategy. The protein complexes were concentrated and stored at −80° C. until use.

DHSO cross-linking of TcdB-CSPG4^{ECD}. The purified TcdB-CSPG4^{ECD} complex (35 μl, 5 μM) was cross-linked with 65 mM DHSO (dihydrazide sulfoxide) and 65 mM 4-(4,6-Dirnethoxy-1,3,5-triazin-2-yl)-4-methylmorpholinium chloride (DMTMM) in PBS (pH 7.4) for 1 h at room temperature. The resulting cross-linked products were subjected to enzymatic digestion using a FASP (Filter Aided Spampie Preparation) protocol. Briefly, cross-linked proteins were transferred into Millipore Microcap Ultracel PL-30 (30-kDa filters), reduced/alkylated, and digested with Lys-C/trypsin. The resulting digests were desalted and fractionated by peptide size-exclusion chromatography (SEC). The fractions containing DHSO cross-linked peptides were collected for subsequent LC MSⁿ analysis. Three biological replicates were performed to obtain highly reproducible cross-link data.

LC MSⁿ analysis of DHSO cross-linked peptides. LC MSⁿ analysis was performed using a Thermo Scientific Dionex ULtiMate 3000 system online coupled with an Orbitrap Fusion Limos mass spectrometer. A 50 cm×75 μm Acclaim PepMap C18 column was used to separate peptides over a gradient of 1 to 25% ACN in 106 Min at a flow rate of 300 nl/min. Two different types of acquisition methods were utilized to maximize the identification of DHSO cross-linked peptides: (1) top four data-dependent MS³ and (2) targeted MS³ acquisition optimized for capturing DHSO cross-linked peptides by utilizing the mass difference between characteristic MS² fragment ions of DHSO cross-linked peptides (α−β) (that Δ=α_{T}−α_{A}=β_{T}−β_{A}=31.9721 Da).

Data analysis and identification of DHSO cross-linked peptides. MSⁿ data extraction and analysis were performed. MS³ data were subjected to Protein Prospector (v.5.19.1) for database searching, using Batch-Tag against a custom database containing nine protein entries concatenated with its random version. The mass tolerances were set as ±20 ppm and 0.6 Da for parent and fragment ions, respectively. Trypsin was set as the enzyme with three maximum missed cleavages allowed. Cysteine carbamidomethylation was set as a fixed modification. Variable modifications included protein tea-terminal acetylation, methionine oxidation, and N-terminal conversion of glutamine to pyroglutamic acid. Additionally, three defined modifications on glutamic and aspartic acids were chosen, which included alkene (C₃H₄N₂, +65 Da), suifenic acid (C₃H₆N₂S; +118 Da), and thiol (C 3 H 4 N 2 S; +100 Da), representing cross-linker fragment moieties. Only a maximum of four modifications on a given peptide was avowed during the search. The in-house program XI-tools was used to identify, validate, and summarize cross-linked peptides based on MSⁿ data and database searching results. Following integration of MSⁿ data, no cross-links involving decoy proteins were identified. Only cross-linked peptides that were identified in all three biological replicates are reported.

Electron-microscopy grid preparation and image acquisition, For cryo-EM data collection, 4 μl of purified TcdB^{core}-CSPG4^{mini} complex was applied at a concentration of ˜0.2 mg/ml to glow-discharged holey carbon grids (Quantifoil Grid R2/2 Cu 200 mesh). The grids were blotted for 1.5 second using an FEI Vitrobot plunger at 10° C. and 100% humidity, and then plunge-frozen in liquid ethane cooled by liquid nitrogen. TWo datasets were collected from two grids using similar parameters. For both data collections, cryo-EM imaging was performed on a Titan Krios electron microscope equipped with a Galan K3 direct electron detector and a Galan Image Filter using a slit width of 20 eV. The microscope was operated at 300 keV accelerating voltage, at a magnification of 105 kX in super-resolution mode resulting in a pixel size of 0.415 Å. All images were automatically recorded using SerialEM. For the first dataset, movies were obtained at an accumulated dose of 40 e-/Å² with defocus ranging from −1.2 to −2.2 μm. For the second dataset, movies were obtained at an accumulated dose of 46 e−/Å² with defocus ranging from −1.2 to −2.2 μm. The total exposure time was 2.3 s over 66 frames per movie stack. It was noticed that the first dataset had a preferred orientation problem during data processing. Therefore, a second data set was collected using a grid with a thicker ice layer, which yielded more particles with better orientations.

Image processing and structure determination. All acquired movies underwent patch motion correction and patch CTF estimation in cryoSPARC v2. Particles were auto-picked using a blob picker in cryoSPARC. The following 2D, 3D classifications, and refinements were all performed in cryoSPARC. For each of the two datasets, particles were first extracted with a box size of 896×896 pixels and bin the data by 4. After rounds of 2D classification, 559,247 good particles were obtained by merging the two datasets, which were used for ab-initio reconstruction into 5 classes, followed by further heterogeneous refinement. One of the best classes with clear features was chosen for homogeneous refinement. After non-uniform refinement followed by local refinement with a mask, a 3.37 Å resolution map was obtained, which showed the overall shape of the TcdB^{core}-CSPG4^{mini} complex. Similarly, a box size of 576×576 pixels was also used and bin the data by 3. After rounds of 2D classification, 560,946 good particles were obtained by merging the two datasets, which were used for ab-initio reconstruction into 5 classes, following further heterogeneous refinement. One of the best classes with clear features and best resolution was chosen for homogeneous refinement. After non-uniform refinement followed by local refinement with a tight mask to omit the highly flexible and low resolution region, a 3.17 Å resolution density map was obtained, which was sharpened using local sharpening in Phenix. Using the full length TcdB structure as an input model, a model for the TcdB^{core}-CSPG4^{mini} complex was able to be built using Phenix This initial structure model was used for iterative manual building in Coot and real space refinement in Phenix. Figures were generated using PyMOL (Schrodinger) and UCSF chimera.

Dynamic light scattering assay. Dynamic light scattering (OLS) was performed using a Malvern Instruments Zetasizer Nano series instrument and data were analyzed using Zetasizer Version 7.12 software. 100 μl of the TcdB^{core}-CSPG4^{mini} complex at 0.1 mg/ml was assayed at 25° C. A representative CLS profile from 3 similar results was reported.

Bio-layer interferometry (BLI) assays. The binding affinities between TcdB and Repeat1 were measured by BLI assay using an OctetRED96 (ForteBio). Prior to use, bio-sensors were soaked in the assay buffer (20 mM HEPES, 400 mM NaCl, pH 7.5, 10 mM CaCl₂, 0.1% Tween-20, 0.5% BSA) for at least 10 min. Briefly, Repeat1-Fc (50 nM) was immobilized onto capture biosensors (Dip and Read Anti-higG-Fc, ForteBio) and balanced with the assay buffer. The biosensors were then exposed to different concentrations of TcdB1 or TcdB2, followed by the dissociation in the same assay buffer. Binding affinities (K_{d}) were calculated using the 1:1 binding model by ForteBio Data analysis HT 10.0.

To analyze the competition between bezlotoxumab and CSPG4 on binding to TcdB, the His-tagged Repeat1 (200 nM), which was biotinylated using EZ-Link NHS-PEG4-Biotin (Thermo Fisher Scientific) at pH 6.5, was immobilized onto capture biosensors (Dip and Read Streptavidin, ForteBio) and balanced with the assay buffer. The biosensors were first exposed to TcdB1 or TcdB2 (200 nM), respectively, followed by balanced with the assay buffer. The biosensors were then applied to bezlotoxumab (200 nM), followed by the dissociation in the assay buffer. Reversely, bezlotoxumab (200 nM) was immobilized onto capture biosensors (Dip and Read Anti-hlgG-Fc, ForteBlo) and balanced with the assay buffer. The biosensors were first exposed to TcdB1 or TcdB2 (200 nM), respectively, followed by balanced with the assay buffer. The biosensors were then applied to CSPG4^{mini} (200 nM), followed by the dissociation in the assay buffer

Protein melting assay and size-exclusion chromatography. The thermal stability of TcdB1 variants was measured using a fluorescence-based thermal shift assay on a StepOne real-time PCR machine (Life Technologies). Each protein (˜0.5 mg/ml) was mixed with the fluorescent dye SYPRO Orange (Sigma-Aldrich) and heated from 25° C. to 95° C. in a linear ramp. The midpoint of the protein-melting curve (Tₘ) was determined using the analysis software provided by the instrument manufacturer. Data obtained from three independent experiments were averaged to generate the bar graph. The folding of Repeat1-Fc variants was verified by Superdex-200 size-exclusion chromatography.

Pull-down assays. For the structure-based mutagenesis studies, interactions between TcdB and CSPG4 were examined using pull-down assays using Protein A or Strep-Tactin resins in a binding buffer comprising 20 mM HEPES, pH 7.5, 150 mM NaCl, 10 mM CaCl2, and 0.1% Tween-20. When testing the TcdB variants, Repeat1-FG was used as the bait and TcdB variants (WI and mutants) were the prey. Repeat1-Fc (45 pg) was pre-incubated with Protein A resins at room temperature for 1 h, and the unbound protein was washed away using the binding buffer. The resins were then divided into small aliquots and mixed with TcdB variants (˜4-fold molar excess over Repeat1-Fc). Pull-down assays were carried out at room temperature for 3 h. The resins were then washed twice, and the bound proteins were released from the resins by boiling in SOS-PAGE loading buffer at 95° C. for 5 min. A similar protocol was used to examine the interactions between Repeat1-Fc variants (preys) and the Twin-Strep tagged TcdB1 (bait) immobilized on Strep-Tactin resins, as well as the simultaneous binding of Repeat1-Fc and CRD2 (preys) to the Twin-Strep tagged TcdB1 (bait). CRO2 was expressed and purified. Samples were analyzed by SOS-PAGE and Coomassie Blue staining.

The competition between bezlotoxurnab and CSPG4 on binding to TcdB was examined by two-step pull-down assays using Protein A or Strep-Tactin resins. In the first set of experiments, bezlotoxurnab served as the bait, TcdB1 or TcdB2 was the prey in the first step and CSPG4^{mini} was the prey in the second step. Specifically, bezlotoxurnab (40 μg) was pre-incubated with Protein A resins at 12° C. for 1 h and the unbound protein was washed away. The bezlotoxumab-bound resins were then divided into small aliquots and mixed with ˜2-fold molar excess of TcdB1 ar TcdB2 and the unbound toxins were washed away after 2 h incubation at 12° C. Lastly, CSPG4^{mini} (˜4-fold molar excess over bezlotoxumab) or the blank binding buffer was added to each tube. After incubation at 12° C. for 2 h, the resins were washed twice and the bound proteins were heating released from the resins at 95° C. for 5 min and further examined by 4-20% SOS-PAGE.

In the second set of experiments, 20 μg of biotin labelled CSPG4^{mini} was used as the bait and pre-incubated with Strep-Tactin resins at 12° C. for 1 h. The unbound protein was washed away and the CSPG4^{mini}-bound resins were then divided into small aliquots. TcdB1 or TcdB2 (˜2-fold molar excess over CSPG4^{mini}) were the preys in the first step and bezlotoxurnab (˜4-fold molar excess over CSPG4^{mini}) was the prey in the second step. The two-step pull-down assays were carried out using a protocol similar to the one described above.

C. difficile Infection Assay. All the animal studies were conducted according to ethical regulations under protocols approved by the Institute Animal Care and Use Committee (IACUC) at Boston Children's Hospital (18-10-3794R). Clostridioides difficile infection model has been described previously. C57BL/6 mice were originally purchased from Charles River and a colony was established in the same room hosting CSPG4 KO mice (but two strains were not cohoused in the same caae), CSPG4 KO mice were obtained. Briefly, mice (6-8 weeks, both male and female) were fed with a mixture of antibiotics in water for 3 days (kanarnycin (0.4 mg/mL), gentamicin (0.035 mg/mL), colistin (850 U/mL), metronidazole (0.215 mg/mL), and vancomycin (0.045 mg/mL)), The mice were then fed with normal water for one day, and intraperitoneally injected (i.p. injection) with a single dose of clindamycin (10 mg/kg). One day after the clindamycin injection, animals were challenged with the PBS control or C. difficile spores (1×10⁵ or 1×10⁴ per mouse) and monitored twice daily for 48 h. Symptoms such as diarrhea, body weight loss, and behavior changes were recorded. Animals were euthanized with CO₂ asphyxiation when animals were moribund; or animals had weight loss of or greater than 15% body weight. All live mice at 48 h were euthanized to harvest the cecum and colon tissues, which were subjected to either hematoxylin and eosin (H&E) staining for histological score analysis or immunofluorescence staining for Claudin-3.

Preparation of C. difficile spores. Briefly, C. difficile was recovered from a −80° C. freezer with Brain Heart Infusion medium (Fischer Scientific) plus 5% yeast extract (BD Difco), and cultured for 24 h at 37° C. in an anaerobic chamber until stationary phase. C. difficile culture was then spread out on 70:30 plates with a cotton swab. Spores were harvested and purified with 50% ethanol after 14-day growth and sporulation, and frozen at −30° C. for storage.

Hematoxylin and Eosin (H&E) staining for histology analysis and immunofluorescence staining. Briefly, the cecum or colon tissues were washed with PBS until the contents were removed completely. The tissues were fixed in 10% phosphate buffered formalin for 24 h, embedded in paraffin, and sectioned 6 μm each. Histology analysis was carried out with H&E staining. Stained sections were scored by two observers blinded to experimental groups, based on 4 criteria including inflammatory cell infiltration, hemorrhagic congestion, epithelial disruption, and submucosal edema on a scale of 0 to 3 (normal, mild, moderate, or severe). The total histological scores were the addition of scores from the four criteria. Immunofluorescence analysis of Claudin-3 was carried out using rabbit polyclonal anti-Claudin-3 (Abeam, ab15102, 1:100) antibody. The images were taken by Olympus microscopy IX51 (software cellSens standard 1.15) and Zeiss microscopy (software Zen 2.5).

Cell cytopathic rounding assay. The cytopathic effect (cell rounding) of WT and mutated TcdB was analyzed by standard cell-rounding assay. Briefly, cells were exposed to a gradient of TcdB and TcdB mutants for 6 and 24 h. The phase-contrast images of cells were taken (Olympus IX51, 10 ˜20× objectives). The numbers of round shaped and normal shaped cells were counted manually. The percentage of round shaped cells was plotted and fitted using the GraphPad Prism software. CR₅₀ is defined as the toxin concentration that induces 50% of cells to be rounded in 24 h. Data were represented as mean ±s.d. from three independent biological replicates.

Cell surface binding assay. Binding of WT and mutated TcdB to cells was analyzed by the cell surface binding assay. Briefly, cells were exposed to TcdB (10 nM) or TcdB mutants (10 nM) for 1 0 min at room temperature. Cells were washed three times with PBS and lysed with RIPA buffer (50 mM Tris, 1% NP₄O, 150 mM NaCl, 0.5% sodium deoxycholate, 0.1% SOS, with a protease inhibitor cocktail (Sigma-Aldrich). Cell lysates were centrifuged and supernatants were subjected to western blotting using chicken polyclonal anti-TcdB IgY (List Labs, #754A, 1:2000) and goat anti-chicken IgY H&L (HRP) (Abeam, ab97135, 1:2000) antibodies to examine the binding of TcdB mutants, Chicken polyclonal anti-actin antibody (Ayes Labs, ACT-1010, 1:2000) was used for negative control.

Cecurn injection assay. The in vivo toxicity of WT and mutated TcdB was tested by the cecum injection assay. Briefly, mice (CD1, 6-8 weeks, both male and female, purchased from Envigo) were fasted 19 h and then deeply aestheticized with 3% isoflurane. A midline laparotomy was performed, and 100 μL of PBS, TcdB (6 μg) or TcdB mutant (6 μg) was injected across the ileocecal valve into the cecal lumen via an insulin syringe (31G). The incision was closed with absorbable suture (5-0 Vicryl). The cecum was harvested after a 6 h recovery period. Tissues were fixed in 10% formalin, paraffin-embedded, sectioned, and subjected to either hematoxylin and eosin (H&E) staining for histological score analysis or immunofluorescence staining for Claudin-3.

in vitro protection assay. The in vitro protection efficacy of inhibitors was tested by the cytopathic rounding effect, Briefly, TcdB1 (10 pM) or TcdB2 (100 pM) were pre-incubated with 2-fold serial-diluted inhibitors in DMEM medium (with 3 mM CaCl₂) at 37° C. for 2 h. Cells were then exposed to the toxin, or toxin-inhibitor mixture, for the indicated time. The phase-contrast images of cells were taken (Olympus IX51, 10 ˜20× objectives). The numbers of round shaped and normal shaped cells were counted manually. The percentage of round shaped cells was plotted and fitted using the GraphPad Prism software. Data were represented as mean ±s.d. from three independent biological replicates.

In vivo protection assay. The in vivo protection efficacy of inhibitors was tested by The cecum injection assay. Briefly, TcdB1 (6 μg) and TcdB2 (6 μg) were premixed with Repeat1-Fc (30 pg) or bezlotoxumab (52 μg). The PBS control, toxin, toxin with Repeat1-Fc Or beziotoxmab, or the Repeat1-Fc control was injected into the connection part between ileum and cecum, following fasting and anesthesia of CD1 mice. The cecum tissue of animals was harvested after 6-h recovery, and subjected to hematoxylin and eosin (H&E) staining for histological score analysis.

Colony Forming Units (CFU) quantification during the infection. The CFU/g feces of C. difficile and the TcdB titer/g feces of infected mice were quantified. Briefly, the mice were fed with antibiotic water for three days. Regular water was resumed for one day, followed with i,p. injection of one dose of clindamycin (10 mg/kg). C. difficile spores (1×10⁴ per mouse) were administered via oral gavage 24 h after the clindamycin injection, Feces were collected (at 24, 48, and 72 h after infection), weighted, and frozen at −80° C. immediately until ready to use. For CFU counting, feces were completely dissolved in 500 μL PBS plus 500 μL 95% ethanol and sat for 1 h at room temperature. Dissolved feces were then serial diluted and plated on C. difficile selected plates (CHROMID® C. DIFFICILE, BioMérieux). C. difficile spores were incubated 24 h at 37° C. anaerobically, and CFU was counted manually and standardized to per gram feces.

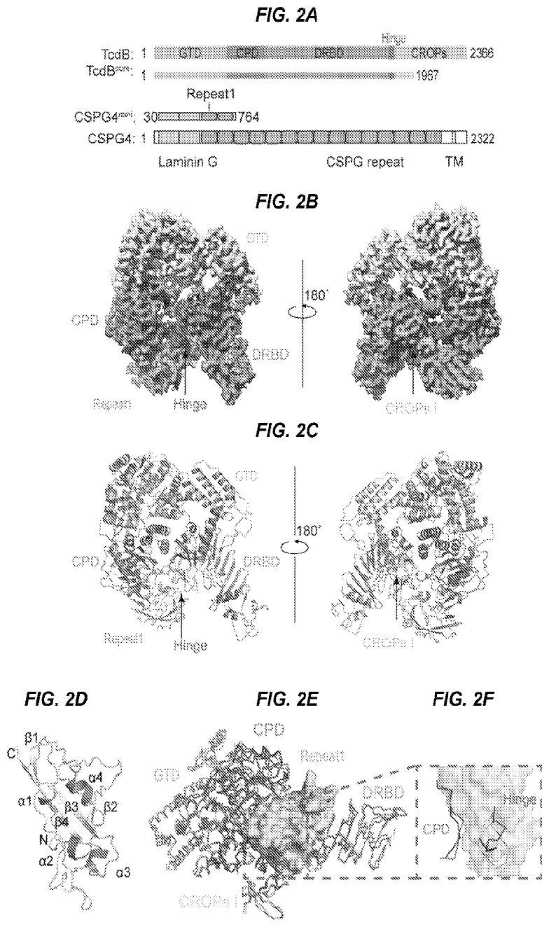

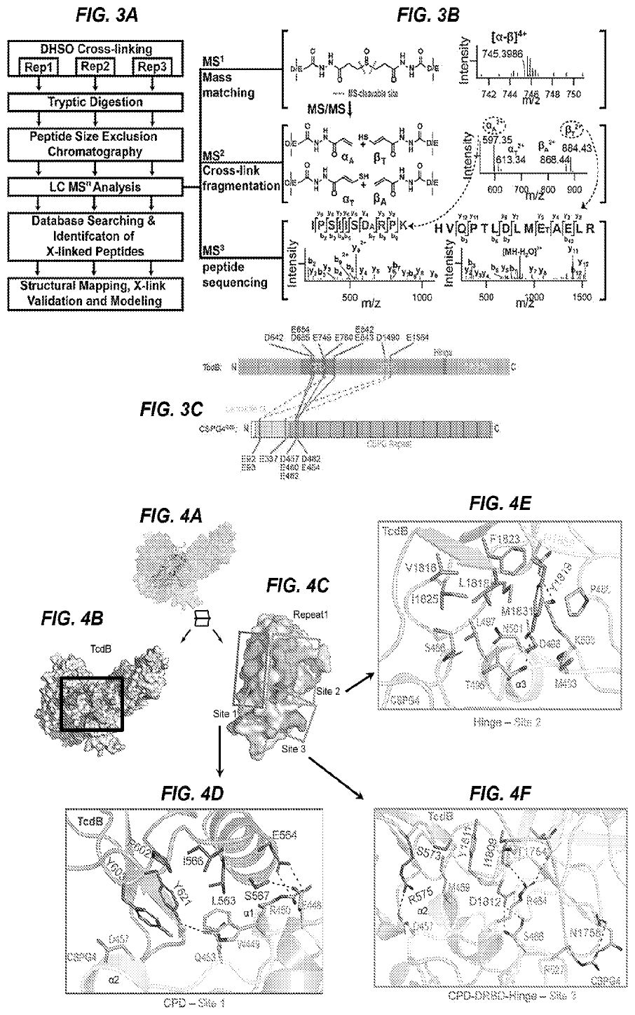

Structure determination of the TcdB-CSPG4 complex by cryo-EM. CSPG4 is a large highly glycosylated single transmembrane protein (˜251 kDa). Its extracellular domain was predicted to contain a signal peptide, two laminin G motifs, and 15 consecutive CSPG repeats (FIG. 2A). Initial efforts using the recombinant full extracellular domain of human CSPG4 (residues 30-2204, referred to as CSPG4^{ECD}) and TcdB1 holotoxin (VPI 10463 strain) were hampered by the structural flexibility of TcdB and CSPG4. First, it was sought to define the interacting domains within TcdB1 and CSPG4^{ECD} employing crosslinking mass spectrometry (XL-MS) using the MS-cleavable crosslinker dihydrazide sulfoxide (DHSO) (FIGS. 3A and 3B). When forming a complex, acidic residues in TcdB1 and CSPG4^{ECD} that have Cα-Cα distances within 35 Å can be cross-linked by DHSO, and the resulting cross-linked peptides could be identified using multistage mass spectrometry (MSⁿ).

A total of 263 unique DHSO cross-finked peptides of the TcdB1-CSPG4ECD complex (Table 3) were identified, representing 18 inter-protein and 245 intra-protein (167 in TcdB1 and 78 in CSPG4^{ECD}) cross-links. The intramolecular cross links in TcdB1 show good correlations with the crystal structure of TcdB1 holotoxin. Fourteen pairs of the inter-protein cross links were mapped to the first predicted CSPG repeat and the CPD and the N-terminus of DRBD of TcdB, indicating direct interactions between them (FIG. 3C). The rest 4 pairs of cross links suggested that the laminin G domains of CSPG4 may adopt flexible conformations and could transiently move within ˜35 Å of the CPD or DRBD of TcdB, because the same residues (e.g., E92/E93) in this region of CSPG4 could be cross-linked to amino acids on the CPD and DRBD of TcdB that are 97 Å away from each other (Table 3). Guided by the XL-MS results, interactions between a number of fragments of TcdB1 and CSPG4 and their biochemical behaviors were analyzed, and narrowed down a fragment of TcdB1 (residues 1-1967, referred to as TcdB^{core}) that contains the GTD, CPD, DRBD, and the first unit of CROPs (termed CROPs I), which could robustly bind to an N-terminal CSPG4 fragment composed of two laminin G motifs and first two CSPG repeats (residues 30-764, referred to as CSPG4^{mini}) (FIG. 2A and Table 4).

TABLE 3
Linkages between TcdB and CSPG4 identified by XL-MS.

| | Total | Distance |
|---|---|---|
| Linkages | IDs | (Å) |
| TcdB:D642-CSPG4:E460 | 291 | 11.6 |
| TcdB:D642-CSPG4:E462 | 199 | 16.4 |
| TcdB:E843-CSPG4:E460 | 62 | 20.5 |
| TcdB:D685-CSPG4:E460 | 37 | 20.1 |
| TcdB:E843-CSPG4:E462 | 37 | 22.8 |
| TcdB:D642-CSPG4:D457 | 36 | 13.5 |
| TcdB:D685-CSPG4:E462 | 31 | 25.2 |
| TcdB:E684-CSPG4:E460 | 23 | 23.8 |
| TcdB:E684-CSPG4:E462 | 20 | 28.8 |
| TcdB:E842-CSPG4:E460 | 20 | 23.1 |
| TcdB:E1564-CSPG4:E337* | 19 | N/A* |
| TcdB:E842-CSPG4:E462 | 16 | 25.1 |
| TcdB:D1490-CSPG4:E93* | 12 | N/A* |
| TcdB:D1490-CSPG4:E92* | 8 | N/A* |
| TcdB:E749-CSPG4:D484 | 7 | 18.7 |
| TedB:E749-CSPG4:E482 | 6 | 23.4 |
| TcdB:E843-CSPG4:D457 | 4 | 24.3 |
| TcdB:E760-CSPG4:E92* | 3 | N/A* |

*These linkages have no known distance as the structure of the N-terminal laminin G motifs have not been determined due to high flexibility.

TABLE 4
Characterization of various TcdB and CSPG4 truncations.

| TcdB | | | CSPG4 | | |
|---|---|---|---|---|---|
| Frag- | E. coli | CSPG4 | Frag- | HEK293F | TcdB |
| ment | Expression | Binding * | ment | Expression | Binding * |
| 1-2366 | Yes | Yes | 30-2204 | Yes | Yes |
| 1-2099 | Yes | Yes | 30-2148 | No | N/A |
| 1-1967 | Yes | Yes | 30-2028 | No | N/A |
| | | | 30-1465 | No | N/A |
| | | | 30-764 | Yes | Yes |
| | | | 410-551 | Yes | Yes |
| | | | 420-645 | No | N/A |

* The binding between TcdB and CSPG4 fragments was examined by pull down assays.

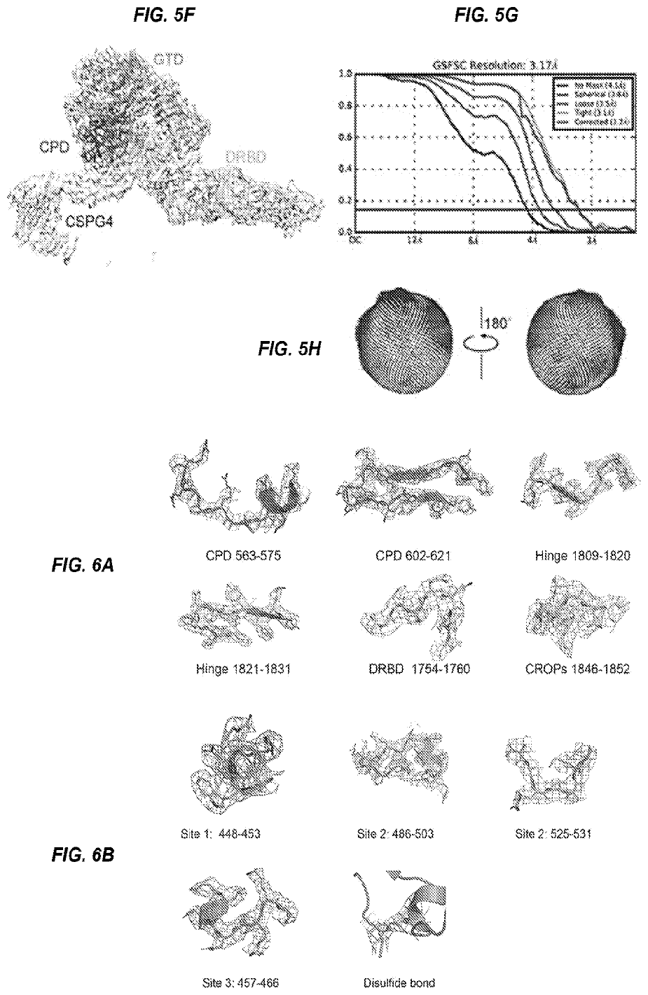

A stable complex composed of TcdB^{core} and CSPG4^{mini}, which was used for cryo-EM study (FIG. 5A-5D). The preliminary data analysis yielded a 3.4 Å resolution structure for the TcdB^{core}-CSPG4^{mini} complex, which revealed that CSPG4^{mini} binds to a groove in TcdB that is surrounded by the CPD, DRBD, hinge, and CROPs I (FIGS. 5E and 5F). which is consistent with the XL-MS studies. 3D variability analysis indicated that the distal region in the DRBD of TcdB and the N terminal two laminin G motifs of CSPG4^{mini} were highly flexible, which hindered the ability to obtain a high-resolution map for de novo model building. Notably, these flexible regions in TcdB and CSPG4 were outside the complex interface. Therefore, the resolution could be improved by using a smaller box size during particle picking to focus on the TcdB-CSPG4 interface, With a focused refinement, the density map was further improved to 3.17 Å resolution that allowed de novo model building for CSPG4, while the TcdB structure was built using the crystal structure of TcdB holotoxin as a model 10 (FIGS. 2B and 2C and FIGS. 5G and 5H). Structure determination statistics and representative density maps for the protein complex were shown in Table 5 and FIGS. 6A and 6B.

TABLE 5
Cryo-EM data collection, refinement, and validation statistics.

| | TcdB^{core}-CSPG4^{mini} | |
|---|---|---|
| | (EMDB: EMD-23909) | |
| Data collection and | (PDB: 7ML7) | |
| processing | Data Set #1 | Data Set #2 |
| Magnification | 105 K | 105 K |
| Voltage (kV) | 300 | 300 |
| Electron exposure (e−/Å²) | 40 | 46 |
| Defocus range (μm) | −1.2 to −2.2 | −1.2 to −2.2 |
| Pixel size (Å) | 0.415 | 0.415 |
| Symmetry imposed | C1 | C1 |
| Initial particle images (no.) | 5,425,209 | 3,292,851 |
| Final particle images (no.) | 177,995 (Combined Data Sets) | |
| Map resolution (Å) | 3.17 | |
| FSC threshold | 0.143 | |
| Refinement | | |
| Initial model used (PDB code) | 6OQ5 | |
| Model resolution (Å) | 3.4 | |
| FSC threshold | 0.5 | |
| Map sharpening B factor (Å²) | −55.2 | |
| Model composition | | |
| Non-hydrogen atoms | 10,913 | |
| Protein residues | 1,354 | |
| Ligands(Zn) | 1 | |
| B factors (Å²) | | |
| Protein | 63.26 | |
| Ligand | 81.76 | |
| R.m.s. deviations | | |
| Bond lengths (Å) | 0.004 | |
| Bond angles (°) | 0.743 | |
| Validation | | |
| MolProbity score | 1.80 | |
| Clashscore | 5.73 | |
| Poor rotamers (%) | 0.48 | |
| Ramachandran plot | | |
| Favored (%) | 92.10 | |
| Allowed (%) | 7.82 | |
| Disallowed (%) | 0.07 | |

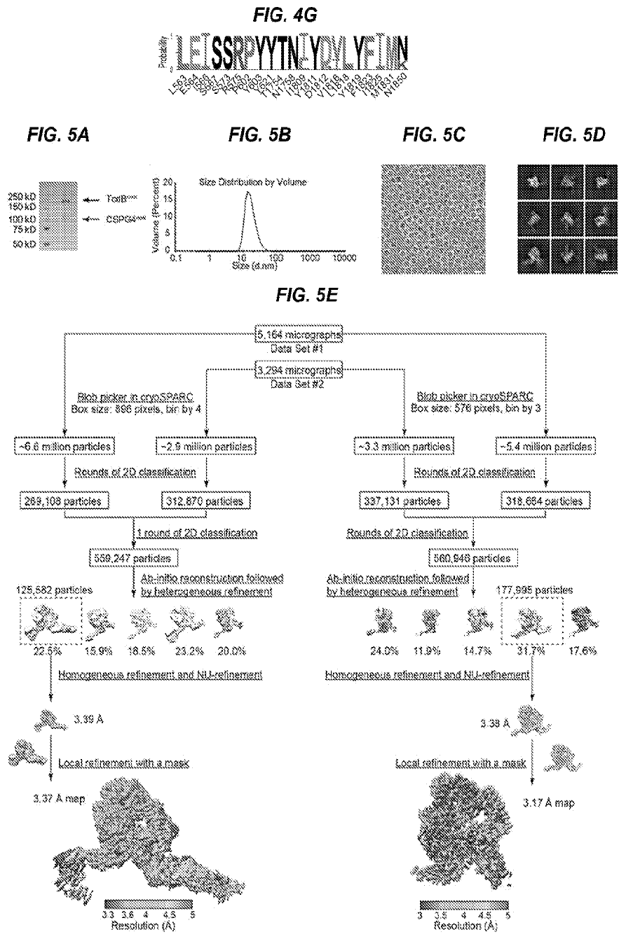

TcdB1 ar d TcdB2 use a conserved composite binding site for CSPG4. The structure of the TcdB-CSPG4 complex reveals that the first CSPG repeat of CSPG4 (termed Repeat1, residues 410-551) is mainly responsible for TcdB binding, while the rest of CSPG4 pointing away from the toxin (FIG. 5F). Repeat1 has a compact structure consisting of a four-strand β sheet and 4 short α helices, which are connected by intermittent loops and stabilized by a disulfide bridge (FIG. 2D). Despite its small size, Repeat1 directly interacts with many amino acids that are dispersed across over 1,300 residues on the primary sequence of TcdB, including the CPD, DRBD, hinge, and CROPs (FIGS. 2B and 2C). All these TcdB residues converge spatially to form a composite binding site for Repeat1 involving an extensive interaction network and burying a large molecular interface between them (˜2.715.5 Å²) (FIG. 4A, 4B, 4C). This unusually complex binding mode, especially the involvement of the CPD, is unexpected, because it was believed that the receptor binding of TcdB is carried out by the DRBD or the CROPs.

More detailed structural analysis showed that the TcdB-binding surface in Repeat1 could be divided into three subsites (FIG. 4C). The site-1 of Repeat1 (residues 448-457) binds to the CPD via hydrogen bonds, charge-charge interaction, as well as a large patch of hydrophobic interactions (FIG. 4D and Table 6. The site-2 of Repeat1 (residues 466-503) binds to the hinge of TcdB involving mainly hydrophobic interaction and two hydrogen bonds, and also interacts with the CROPs I with a hydrogen bond (FIG. 4E and Table 6). The site-3 of Repeat1 is composed of two separated areas including residues 457-466 and an additional residue (R527) in a nearby loop. It binds to a composite interface in TcdB, which is composed of residues in the CPD, DRBD, and hinge (FIG. 4F and Table 6). CSPG4 is predicted to have fifteen N-linked glycosylation sites with one in Repeat1 (N427) and a single chondroitin sulfate modification at S995 25. We did not observe density for the N427 glycan and it remains to be determined whether these glycans in CSPG4 may contribute to TcdB recognition.

TABLE 6
Protein-protein interactions in the TcdB-CSPG4 complex.

| | TcdB | CSPG4 | Type of |
|---|---|---|---|
| | residues | residues | interaction |
| CPD - Site 1 | E564 | R450 | SB |
| interaction | S567 | E448 | HB |
| | L563 | W449 | vdW |
| | I566 | D457 | |
| | S567 | | |
| | Y621 | | |
| | Y603 | | |
| | P602 | | |
| | Y621 | Q453 | HB |
| Hinge - Site 2 | V1816 | S466 | vdW |
| interaction | L1818 | L497 | |
| | Y1819 | T495 | |
| | F1823 | M493 | |
| | I1825 | P486 | |
| | M1831 | K503 | |
| | | N501 | |
| | Y1819 | D498 | HB |
| | | T495 (mc) | HB |
| | N1850 | K503 | HB |
| CPD-DRBD-Hinge - | R575 | M459 | vdW |
| Site 3 interaction | S573 | | |
| | Y1811 | | |
| | R575 | D457 | SB |
| | T1754 (mc) | R464 | HB |
| | I1809 (mc) | | HB |
| | N1758 | R527 | HB |
| | N1758 (mc) | | HB |
| | D1812 | S466 | HB |
| | | S466 (mc) | HB |

“SB”, “vdW”, and “HB” stand for salt bridge, van der Waals interaction, and hydrogen bond, respectively. “mc” indicates the main-chain-mediated contacts, and all the other contacts are mediated by side-chain atoms.

The overall structure of the CSPG4-bound TcdB^{core} is similar to the crystal structure of TcdB holotoxin with a root-mean-square deviation (r.m.s.d) between comparable Co atoms about 1.06 Å (FIG. 2E). Nevertheless, CSPG4 binding triggers local structural changes in TcdB involving residues 1803-1812 in the hinge and 569-577 in the CPD (FIG. 2F). It is worth noting that the hinge is located at a strategic site in TcdB communicating with all four major domains, and the CROPs of TcdB adopts dynamic conformations relative to the rest of the toxin. Therefore, conformational chanties in TcdB could affect the structure of the hinge and the configuration of the CSPG4-binding site that would subsequently influence CSPG4 binding, while CSPG4 binding could in turn modulate TcdB structure.

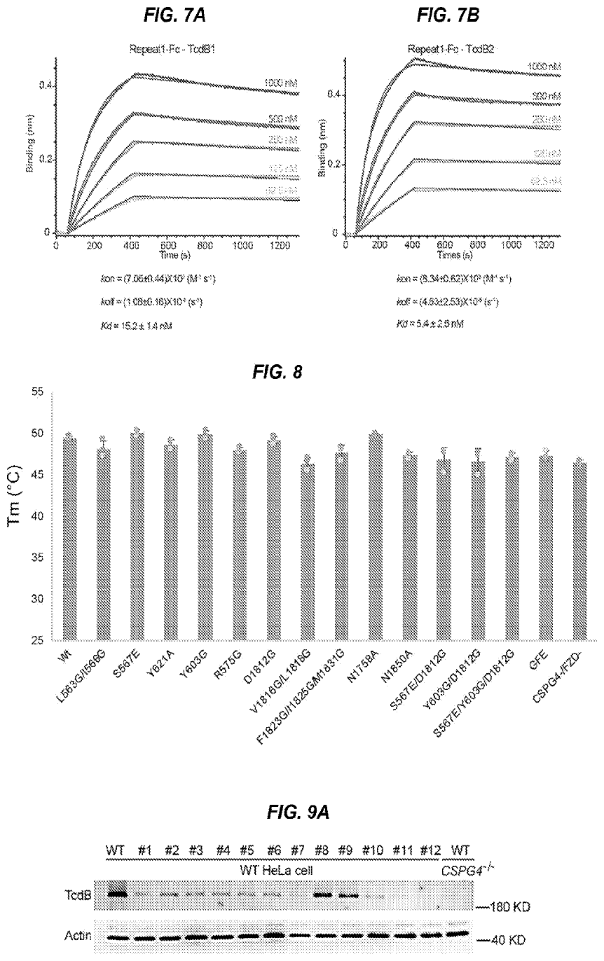

Further, a real-time analysis of the kinetics of TcdB-CSPG4 interactions was carried out using bio-layer interferometry (BLI), For this study, a recombinant CSPG4 Repeat1 that is fused to the N-terminus of the Fc fragment of a human immunoglobulin (Ig) G1 (Repeat1-Fc) was designed. Based on the structural modeling, the Fc fragment in Repeat1-Fc does not interfere with TcdB binding, and provides a convenient way for immobilization of Repeat1-Fc to the biosensors. TcdB1 recognized Repeat1-Fc with a high affinity (dissociation constant, K_{d}˜15.2 nM) (FIG. 7A). Notably, Repeat1-Fc binds to TcdB with a relatively slow on-rate (kₒₙ˜7.06×103 M−1 s−1), which is likely due to organization of multiple structural units in TcdB to form the composite binding site for CSPG4. Nevertheless, once Repeat1 is engaged with TcdB, the complex is very stable as evidenced by their slow binding off-rate (k_{off}˜1.08×10⁻⁴ s⁻¹).

Since TcdB1 and TcdB2 have different primary sequences and pathogenicity, structure-based sequence analysis was carried out between them focusing on the CSPG4-binding site, Remarkably, the key amino acids comprising the composite CSPG4-binding site are nearly identical between TcdB1 and TcdB2, even though these residues scatter across multiple TcdB domains (FIG. 4G). It is worth noting that the hinge region has large sequence variations among TcdB isoforms, and the hypervariable sequences in this region are believed to contribute to differences in toxicity and antigenicity of TcdB2 and other less virulent strains. But the CSPG4-binding residues in the hinge are conserved between TcdB1 and TcdB2 except for two conservative substitutions of 11809^{TcdB1} with L1809^{TcdB2} and V1816^{TcdB1} with I1816^{TcdB2}. The only other difference is N1850^{TcdB1} in the CROPs I that forms a hydrogen bond with K503 of CSPG4 is replaced with K1850^{TcdB2}. Nevertheless, the BLI binding studies showed that TcdB2 binds to Repeat1-Fc with a high affinity that is even slightly better than TcdB1 (K_{d}˜5.4 nM, kₒₙ˜8.34×10³ M⁻¹ s⁻¹, k_{off}˜4.63×10⁻⁵ s⁻¹) (FIG. 7B). Therefore, the three residue substitutions in the CSPG4-binding site are well tolerated in TcdB2. These data demonstrate that the CSPG4-binding mode is conserved between TcdB1 and TcdB2.

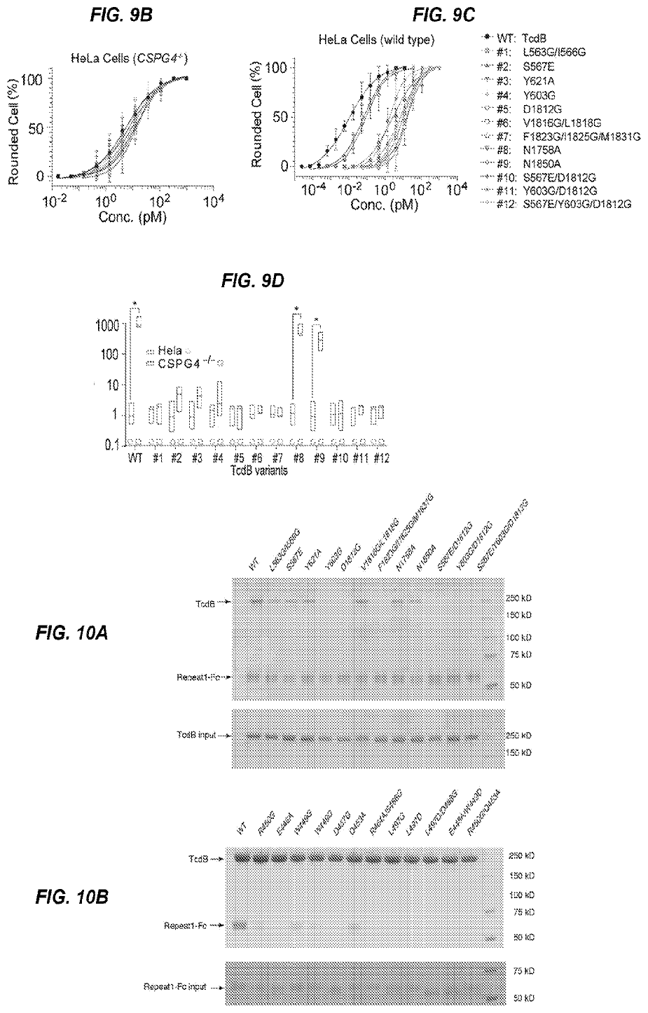

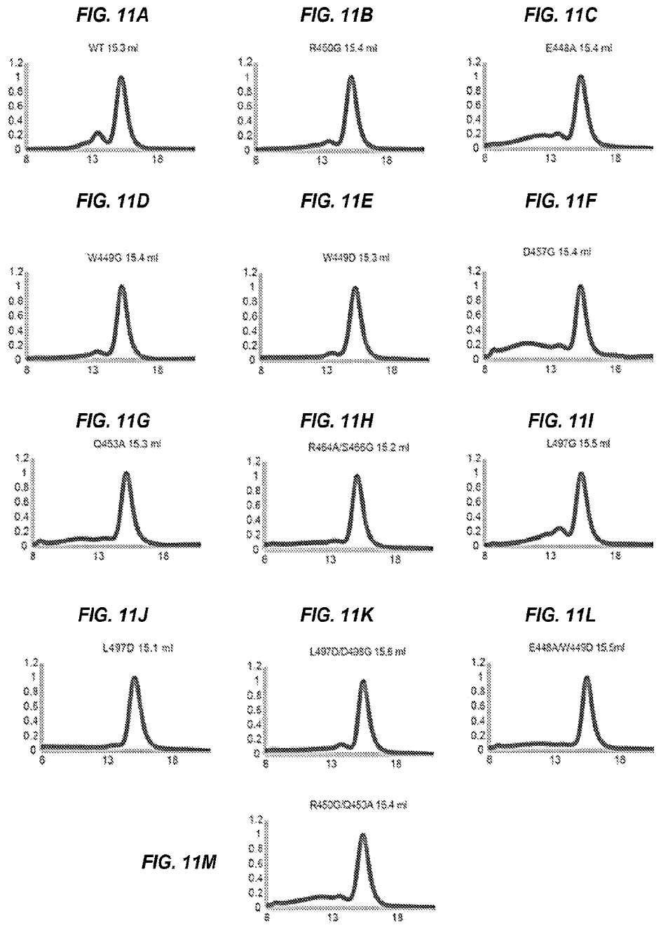

Site-specific mutagenesis to validate TcdB-CSPG4 interactions. Next structure-guided mutagenesis of TcdB1 and CSPG4 was carried out to validate the binding interface and to define loss-of-function mutations in TcdB that could selectively abolish CSPG4 binding. Nine mutations of TcdB1 holotoxin were designed and characterized, where the key CSPG4-binding residues in the CPD (L563G/I566G, S567E, Y621 A, or Y603G), the hinge (D1812G, V1816G/L11818G, or F1823G/I1825G/M1831G), the DRB© (N1758A), or the CROPs I (N1850A) were mutated (FIG. 8). These TcdB1 mutants showed reduced binding to HeLa cells expressing endogenous CSPG4 (FIG. 9A). TcdB-N1758A and N1850A showed the least reduction of binding, suggesting that these two mutations, located in the DRBD and the CROPs respectively, have relatively weaker impact on TcdB-CSPG4 interactions compared with mutations in the CPD or the hinge. Then three combinational mutations of TcdB were designed to simultaneously disrupt the anchoring points for CSPG4 in both the CPD and the hinge, including S567E/D1812G, Y603G/D1812G, and S567E/Y603G/D1812G, and found them largely abolished binding of TcdB to cells. Similar results were confirmed using pull-down assays with Repeat1-Fc as the bait and TcdB variants as preys (FIG. 10A). Variants of CSPG4 Repeat1 were also designed and characterized that carried site-specific mutations in the TcdB-binding interface, including mutations in site-1 (R450G, E448A, W449G, W449D, Q453A, E448A/W449D, R450G/Q453A), site-2 (L497G, L497D, L497G/D498G), and site-3 (D457G, R464A/S466G) (FIG. 11A-11M). These mutations effectively disrupted the binding of TcdB holotoxin to Repeat1 based on pull down assays (FIG. 10B).

How these TcdB mutations affect CSPG4-mediated cytopathic toxicity at functional levels were examined using standard cell-rounding assays, where TcdB entry would inactivate Rho GTPases and cause the characteristic cell rounding phenotype. The concentration of TcdB that induces 50% of cells to be round is defined as cell-rounding 50 (CR₅₀), which is utilized to compare the potency of TcdB variants on the wild-type (WT) HeLa cells that express both CSPG4 and FZDs or the CSPG4 knockout (KO) HeLa cells. As shown in FIG. 9B, all 12 mutant TcdB1 induced cell-rounding with potencies similar to TcdB1 on CSPG4 KO cells, demonstrating that these mutations were properly folded and did not affect FZD-mediated binding and entry of toxins. In contrast, these mutant toxins showed various reduced potencies on WI HeLa cells compared with TcdB1 (FIG. 9C). More specifically, WT TcdB1 showed over 600-fold reduced toxicity on CSPG4 KO cells compared with WT cells, while the toxicity of TcdB1 variants carrying L563G/I566G, D1812G, V1816G/L1818G, F1823G/I1825G/M1831G, and the three combinational mutations were similar on CSPG4 KO cells and WT cells (CR₅₀ ratio ˜1.1-1.3), demonstrating that these mutations effectively and selectively eliminated CSPG4-mediated toxicity on cells (FIG. 9D).

CSPG4 is a physiologically relevant receptor in vivo. Given the extensive structural, in vitro, and ex vivo data demonstrating the role of CSPG4 as a TcdB receptor, it was sought to determine the contribution of CSPG4 to TcdB1 and TcdB2 pathogenicity and its relationship with FZD in vivo using two complementary approaches that were custom designed for Tcd82 and TcdB1, respectively

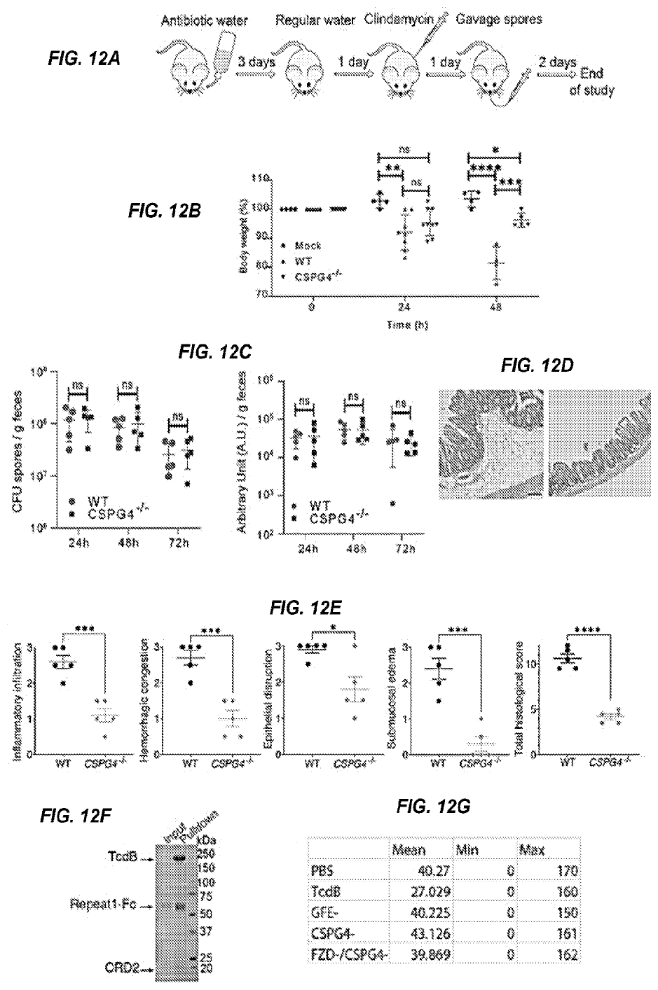

First a C. difficile mutant strain (M7404, tcdA⁻) that only expresses TcdB2 was used to directly assess the contribution of CSPG4 in vivo since Tcd82 does not bind to FZDs. Infection experiments were carried out in mouse models based on established protocols (antibiotic treatment followed with gavage feeding of 1×10⁵ C. difficile spores) (FIG. 12A) to compare pathological development in WT versus CSPG4 KO mice. All mice developed CDI symptoms including diarrhea and body weight loss, but it was less severe in CSPG4 KO mice than the WT mice in general. In addition, infection led to 100% moribundity of WT mice by 48 hours, whereas only 50% of CSPG4 KO mice reached moribundity (FIG. 12B).

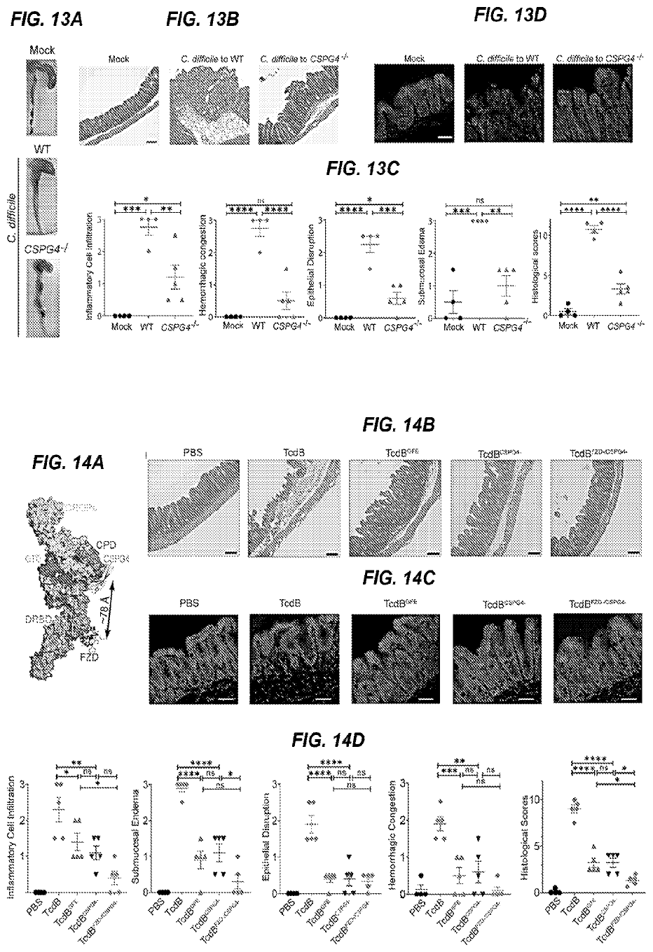

Next, histological analysis of cecum and colon tissues was carried out. There was bloody fluid accumulation in tissues dissected from WI mice after infection, whereas there was much less fluid accumulation in tissues from CSPG4 KO mice (FIG. 13A). Further, histological analysis was carried out with paraffin embedded cecurn tissue sections (FIG. 13B), which were scored based on disruption of the epithelium, hemorrhagic congestion, submucosal edema, and inflammatory cell infiltration, on a scale of 0 to 3 (normal, mild, moderate, or severe, FIG. 13C). Infection induced extensive disruption of the epithelium and inflammatory cell infiltration, as well as severe hemorrhagic congestion and mucosal edema on WT mice (FIG. 130). CSPG4 KO mice showed only moderate levels of epithelium damage and inflammatory cell infiltration, and mild to no hemorrhagic congestion and submucosal edema (FIG. 13B, 13C). Furthermore, TcdB2 induced extensive loss of tight junction in the cecum epithelium from WT mice based on immunofluorescence staining for a tight junction marker Claudin-3, while it was largely intact in CSPG4 KO mice (FIG. 130). Similar results were observed when infection experiments were carried out using a 10-fold lower dose of C. difficile spores (1×10⁴), which did not result in death of mice and thus allowed us to harvest cecurn tissues 90 h after infection (FIG. 12C, 12D, and 12E). Analysis of feces indicated similar levels of C. difficilecolonization and toxin titer in WT and CSPG4 KO mice (FIG. 12C). Taken together, these results demonstrated that CSPG4 is a major receptor for the epidemic TcdB2 in vivo. The residual toxicity of TcdB2 in CSPG4 KO mice indicates that TcdB2 may have unknown low affinity receptor(s).

TcdB1 can be simultaneously bound by CSPG4 and FZD as demonstrated by the cryo-EM structure of the TcdB-CSPG4 complex and the crystal structure of a TcdB-FZD complex, which was confirmed by a pull-down experiment (FIG. 12F). The estimated distance between the centers of CSPG4- and FZD-binding sites in TcdB is about 78 Å, and the two receptors are located on the same side of TcdB, making them possible to simultaneously anchor to the plasma membrane (FIG. 14A). To investigate the relationship of these two receptors for TcdB1, three structure-based rationally designed TcdB1 mutants as molecular tools were used, which carry site-specific mutations to selectively knock out its binding capacity to CSPG4, FZD, or both. Based on the mutagenesis studies described above, TcdB^{S567E/Y803G/D1812G} was chosen as a representative CSPG4 binding deficient TcdB mutant (TcdB^{CSPG4-}). A FZD-binding deficient TcdB variant was previously developed that carries mutations in the FZD-binding site (TcdB^{GFE}). Combining these two TcdB mutants, a unique TcdB variant was generated that is unable to recognize either CSPG4 or FZD (TcdB^{FZD-/CSPG4-}).

The toxicity of these TcdB1 mutants were analyzed in comparison with the WT toxin by directly injecting them into the mouse cecum. This method has the advantage of controlling precisely the amount of toxins and incubation time, in order to capture any differences among these toxins. WT TcdB1 induced severe damage to cecurn tissues, resulting in inflammatory cell infiltration, submucosal edema, epithelial disruption, hemorrhagic congestion, and disruption of tight junction (FIGS. 148, 14C, and 14D and FIG. 12G). Both TcdB^{GFE} and TcdB^{CDPG4-} showed greatly reduced potency, with no significant difference between them: both showed modest levels of inflammatory cell infiltration and submucosal edema, and mild to normal levels of disruption of epithelium, tight junction, and hemorrhagic congestion. TcdB^{FZD-/CSPG4-} showed further reduced toxicity, with minimal levels of disruption to cecum tissues under our assay conditions (FIG. 14D and FIG. 12G). These results demonstrate that FZDs and CSPG4 act as independent receptors in TcdB1 pathogenesis in vivo.

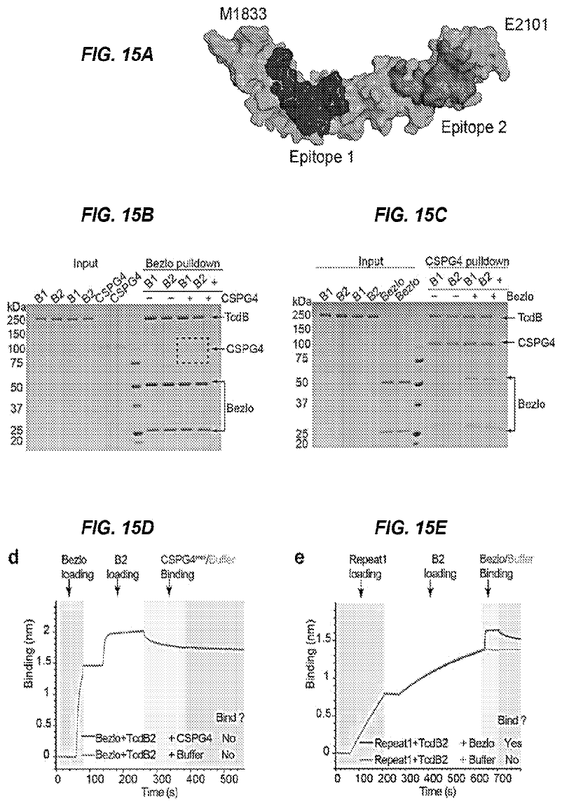

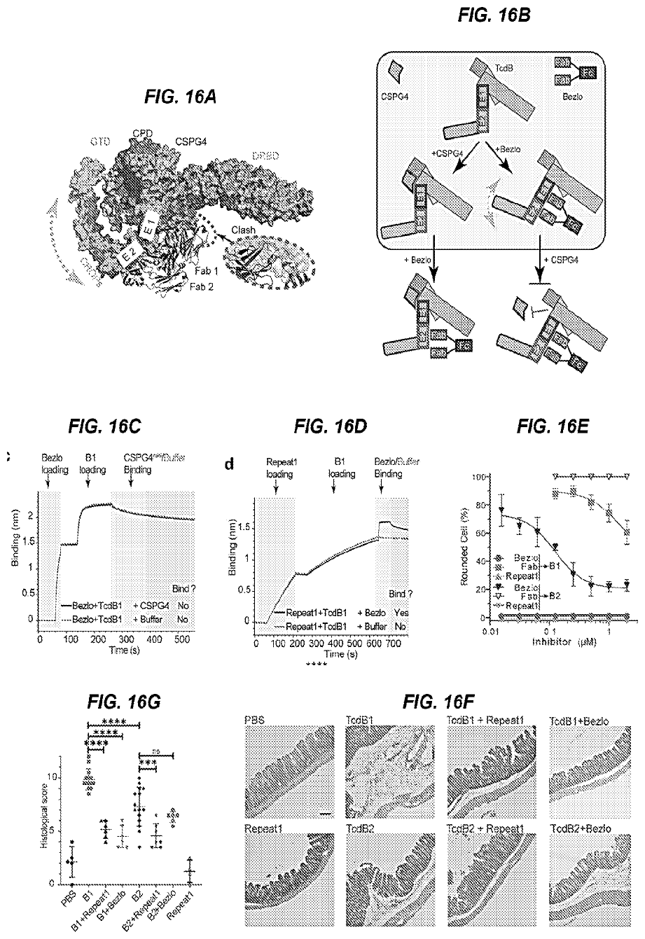

Beziotoxurnab disrupts CSPG4-binding site in an allosteric manner. Bezlotoxumab is the only FDA-approved therapeutic antibody against TcdB, and a prior study suggested that bezlotoxumab reduced binding of TcdB to CSPG4 in vitro in immunoprecipitation assays. However, bezlotoxumab recognizes two closely-spaced homologous epitopes, epitope-1 and epitope-2, in the CROPs (FIG. 15A), which is completely separated from the CSPG4-binding site, and therefore cannot directly compete with CSPG4. Since the prior structural studies were based on bezlotoxumab binding to a fragment of the CROPs, a structural model was generated of bezlotoxumab binding to TcdB holotoxin (FIG. 16A). Bezlotoxumab could bind to the epitope-2 without interfering with the overall structure of TcdB, while its binding to epitope-1 would be hindered by the nearby GTD and DRBD. Therefore, beziotoxurnab has to force the CROPs domain to adopt a different orientation in order to gain access to epitope-1 and occupy both epitopes, which will benefit from the synergy between its two Fab arms (FIG. 16B). Since CSPG4 binds TcdB by simultaneously interacting with the CPD, DRBD, hinge, and CROPs, beziotoxurnab binding may reorient the CROPs relative to the rest of TcdB and compress the CSPG4-binding groove, thus preventing CSPG4 binding in an allosteric manner (FIG. 16B).

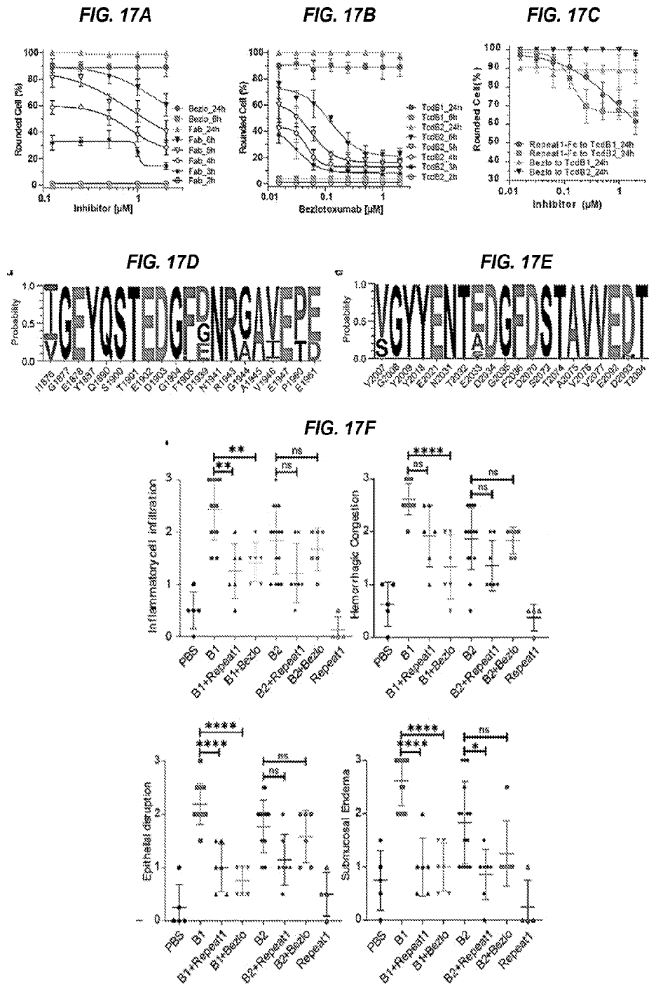

To verify this hypothesis, the competition between bezlotoxumab and CSPG4 was examined using BLI and pull-down assays. When TcdB1 and TcdB2 were pre-bound with the immobilized bezlotoxumab, CSPG4 could not bind subsequently (FIG. 16C and FIGS. 15B and 15D). Meanwhile, the CSPG4-bound TcdB1 and TcdB2 could still bind bezlotoxumab, which is likely due to single-site antibody binding to epitope-2 (FIG. 16D and FIGS. 15C and 15E). To further understand how the single- vs. double-epitope binding modes affect beziotoxurnab's activity, the neutralization potency against TcdB1 was examined for bezlotoxumab and its Fab fragment using the cell rounding assay. When antibodies were pre-incubated with TcdB1 (10 pM) before adding to the culture medium, bezlotoxumab completely protected cells within 6 hours at the lowest concentration tested (16 nM), but its Fab did not show any protection until the concentration reached 2 μM, which only reduced cell-rounding by ˜40% (FIG. 16E and FIGS. 17A and 17B). Without wishing to limit the present invention to any theories or mechanisms it is believed this is due to the lack of synergy on TcdB binding between individual Fab molecules. These data consistently define a unique mechanism for bezlotoxumab at the molecular level, where it relies on synergistic binding to both epitopes in TcdB using its two Fab arms.

However, the need for bezlotoxumab to simultaneously occupy two epitopes in TcdB in order to be effective also increases its susceptibility to residue changes in TcdB variants. Epitope-1 and -2 in TcdB each consists of about 20 amino acids, and variations have been observed in many TcdB variants especially in epitope-1 (FIGS. 17D and 17E). These amino acid substitutions in the bezlotoxumab-binding epitopes are believed to decrease the binding affinities and neutralization potencies of bezlotoxumab. For example, the neutralization efficacy of bezlotoxumab on TcdB2 is ˜200-fold lower than TcdB1. Consistently, bezlotoxumab showed a much lower potency in blocking TcdB2 on HeLa cells compared with TcdB1 in the cell rounding assay, and its Fab failed to show any protection at the highest concentration tested (2 μM) (FIG. 16E and FIG. 17B).

A CSPG4 receptor decoy as a broad-spectrum TcdB inhibitor. As the CSPG4-binding site is conserved between TcdB1 and TcdB2, it is envisioned that Repeat1 could be an effective CSPG4 decoy to block a broad range of TcdB. Thus, the neutralization efficacies of Repeat1-Fc and bezlotoxumab were evaluated against TcdB1 and TcdB2, which represent two largely diverged TcdB isoforms, using cell-rounding assays on HeLa cells, Repeat1-Fc at nM concentrations completely blocked both TcdB1 and TcdB2 within the 6-hour incubation period, whereas bezlotoxurnab only neutralized TcdB1, but not TcdB2 (FIG. 16E), Furthermore, bezlotoxurnab at up to 2 μM failed to block TcdB1 or TcdB2 when incubation time was extended to 24 hours, whereas Repeat1-Fc at the same concentration was still able to partially neutralize TcdB1 and TcdB2 and prevent ˜40% cells from rounding (FIG. 17C). These data demonstrate that Repeat1-Fc offers an enhanced protection against both TcdB1 and TcdB2 than bezlotoxumab.

Repeat1-Fc and bezlotoxumab were further evaluated for blocking TcdB1 and TcdB2 in vivo using the mouse cecum injection model. Briefly, TcdB1 or TcdB2 (6 μg) was pre-incubated with Repeat1-Fc (30 μg) or bezlotoxumab (52 μg), respectively, and the mixture was injected into the mouse cecum. The cecum tissues were dissected out for histological analysis 6 hours later. As shown in FIGS. 16F, and 16G and FIG. 17F, Repeat1-Fc was able to reduce overall damage to cecum tissues from both TcdB1- and TcdB2-treated mice, including less inflammatory cell infiltration, submucosal edema, hemorrhagic congestion, and epithelium disruption, while bezlotoxumab was only effective in reducing TcdB1 toxicity, but showed no effect on TcdB2 under the same assay conditions.

### EXAMPLE 2

The following is a non-limiting example of the present invention. It is to be understood that said example is not intended to limit the present invention in any way. Equivalents or substitutes are within the scope of the present invention.

An 84-year old man is admitted to the hospital after complaining about severe abdominal pain, frequent diarrhea, and a fever lasting for the past two days. After some testing, it is determined that the man has a Clostridium difficile infection. Quickly the man is given a 10 mg/kg body weight intravenous injection of a neutralizing receptor decoy antibody (RDA) that is given during the course of standard-of-care (SOC) COI antibiotic administration such as oral vancomycin or fidaxomicin. After a few days, the man's symptoms subside and after a few days his symptoms have diminished. No side effects are reported. The RDA-treated patients have a lower rate of CDI recurrence than those treated only with antibiotics.

### EXAMPLE 3

The following is a non-limiting example of the present invention. It is to be understood that said example is not intended to limit the present invention in any way. Equivalents or substitutes are within the scope of the present invention.

A nursing home is increasingly noticing that more and more of its residents are becoming infected with a Clostridium difficile infection (CDI). To prevent the spread of CDI any further all the uninfected residents are given a 20 mg/kg body weight intravenous injection of a neutralizing receptor decoy antibody (RDA). After a few days, the amount of residents getting CDI starts to plateau and then slowly decreases. After two weeks of being administered the RDA, the CDI has cleared up. No side effects are reported

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A broad-spectrum neutralizing composition comprising a neutralizing receptor decoy antibody (RDA) that neutralizes a toxin of Clostridium difficile (C. difficile) in various strains of C. difficile, the RDA comprising: a fusion protein comprising a fragment of a Fc region; and a fragment of a chondroitin sulfate proteoglycan 4 (CSPG4) receptor tandemly attached to the Fc region.

2. The composition of claim 1, further comprising a fragment of a frizzled protein (FZD) receptor, wherein the fragment of the FZD receptor comprises a cysteine rich domain (CRD).

3. (canceled)

4. The composition of claim 2, wherein the fragment of the FZD receptor is tandemly attached to the Fc region, such that the CSPG4 receptor fragment and the FZD receptor fragment are on opposite sides of the Fc region, or wherein the fragment of the FZD receptor is tandemly attached to the CSPG4 receptor fragment.

5. (canceled)

6. The composition of claim 2, further comprising a VHH nanobody, wherein the VHH nanobody is tandemly attached to the CSPG4 receptor fragment, the Fc region, or the FZD receptor fragment.

7.-25. (canceled)

26. The composition of claim 1, wherein the toxin is TcdB1 or TcdB2, or both.

27. The composition of claim 1, wherein the RDA mimics a chondroitin sulfate proteoglycan 4 (CSPG4) receptor, a frizzled protein (FZD) receptor, or both.

28.-29. (canceled)

30. The composition of claim 1, wherein the RDA is able to block C. difficile toxin from binding either a chondroitin sulfate proteoglycan 4 (CSPG4) receptor or a frizzled protein (FZD) receptor or both.

31. The composition of claim 1, wherein the RDA is able to neutralize a toxin of C. difficile.

32.-38. (canceled)

39. A method of neutralizing a toxin of C. difficile, the method comprising producing a neutralizing receptor decoy antibody (RDA) composition that binds to C. difficile toxin and blocks it from binding to cell surface receptors, wherein the RDA composition comprises: a fusion protein comprising a Fc region fragment, and a fragment of a chondroitin sulfate proteoglycan 4 (CSPG4) receptor tandemly attached to the Fc region.

40. The method of claim 39, wherein the RDA composition further comprises a fragment of a frizzled protein (FZD) receptor, wherein the fragment of the FZD receptor comprises a cysteine rich domain (CRD).

41. (canceled)

42. The method of claim 40, wherein the fragment of the FZD receptor is tandemly attached to the Fc region, such that the CSPG4 receptor fragment and the FZD receptor fragment are on opposite sides of the Fc region, or wherein the fragment of the FZD receptor is tandemly attached to the CSPG4 receptor fragment.

43. (canceled)

44. The method of claim 40, wherein the RDA composition further comprises a VHH nanobody, wherein the VHH nanobody is tandemly attached to the CSPG4 receptor fragment, the Fc region, or the FZD receptor fragment.

45.-47. (canceled)

48. The method of claim 39, wherein the toxin is TcdB1, TcdB2, or both.

49. The method of claim 39, wherein the RDA mimics a chondroitin sulfate proteoglycan 4 (CSPG4) receptor, a frizzled protein (FZD) receptor or both; wherein the RDA is able to block C. difficile from binding either a chondroitin sulfate proteoglycan 4 (CSPG4) receptor or a frizzled protein (FZD) receptor or both.

50. (canceled)

51. A method of treating a Clostridium difficile infection (CDI) in a patient in need thereof, the method comprising the steps of:
a) administering a standard of care (SOC) antibiotic; and
b) administering a therapeutically effective dose of a neutralizing receptor decoy antibody (RDA) composition; wherein the RDA composition comprises a fusion protein comprising a Fc region fragment; and a fragment of a chondroitin sulfate proteoglycan 4 (CSPG4) receptor tandemly attached to the Fc region.

52. The method of claim 51, wherein the RDA composition further comprises a fragment of a frizzled protein (FZD) receptor, wherein the fragment of the FZD receptor comprises a cysteine rich domain (CRD).

53. (canceled)

54. The method of claim 52, wherein the fragment of the FZD receptor is tandemly attached to the Fc region, such that the CSPG4 receptor fragment and the FZD receptor fragment are on opposite sides of the Fc region, or wherein the fragment of the FZD receptor is tandemly attached to the CSPG4 receptor fragment.

55. (canceled)

56. The method of claim 52, wherein the RDA composition further comprises a VHH nanobody, wherein the VHH nanobody is tandemly attached to the CSPG4 receptor fragment, the Fc region, or the FZD receptor fragment.

57.-59. (canceled)

60. The method of claim 51, wherein the SOC antibiotic is vancomycin or fidaxomicin or metronidazole.

61.-62. (canceled)

63. The method of claim 51, wherein the RDA is administered intravenously.

64.-81. (canceled)


SEQUENCE LISTING (47 sequences)

SEQ ID NO: 1 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTVVESAINDTLESFRENLNDPRFDYNKFFRKRMEIIYDKQKNFINYYKAQREENPELIIDDIVKTYLSNEYSKEIDELNTYIEESLNKITQNSGNDVRNFEEFKNGESFNLYEQELVERWNLAAASDILRISALKEIGGMYLDVDMLPGIQPDLFESIEKPSSVTVDFWEMTKLEAIMKYKEYIPEYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGFDVDSLSTEIEAAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKDEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGEVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSLTLQDEKTIKLNSVHLDESGVAEILKFMNRKGNTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYETNNTYPEVIVLDANYINEKINVNINDLSIRYVWSNDGNDFILMSTSEENKVSQVKIRFVNVFKDKTLANKLSFNFSDKQDVPVSEIILSFTPSYYEDGLIGYDLGLVSLYNEKFYINNFGMMVSGLIYINDSLYYFKPPVNNLITGFVTVGDDKYYFNPINGGAASIGETIIDDKNYYFNQSGVLQTGVFSTEDGFKYFAPANTLDENLEGEAIDFTGKLIIDENIYYFDDNYRGAVEWKELDGEMHYFSPETGKAFKGLNQIGDYKYYFNSDGVMQKGFVSINDNKHYFDDSGVMKVGYTEIDGKHFYFAENGEMQIGVFNTEDGFKYFAHHNEDLGNEEGEEISYSGILNFNNKIYYFDDSFTAVVGWKDLEDGSKYYFDEDTAEAYIGLSLINDGQYYFNDDGIMQVGFVTINDKVFYFSDSGIIESGVQNIDDNYFYIDDNGIVQIGVFDTSDGYKYFAPANTVNDNIYGQAVEYSGLVRVGEDVYYFGETYTIETGWIYDMENESDKYYFNPETKKACKGINLIDDIKYYFDEKGIMRTGLISFENNNYYFNENGEMQFGYINIEDKMFYFGEDGVMQIGVFNTPDGFKYFAHQNTLDENFEGESINYTGWLDLDEKRYYFTDEYIAATGSVIIDGEEYYFDPDTAQLVISE

SEQ ID NO: 2 - protein, 142 aa, Homo sapiens
ELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVND

SEQ ID NO: 3 - protein, 522 aa, Homo sapiens
ASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVND

SEQ ID NO: 4 - protein, 158 aa, Homo sapiens
QYNGERGISIPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGTPTPSLLPEFWTSNPQHGGGGY

SEQ ID NO: 5 - protein, 163 aa, Homo sapiens
QFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDGAPALLTTAPPSGLQPGAGGTPGGPGGG

SEQ ID NO: 6 - protein, 141 aa, Homo sapiens
QPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSGGAG

SEQ ID NO: 7 - protein, 2322 aa, Homo sapiens
MQSGPRPPLPAPGLALALTLTMLARLASAASFFGENHLEVPVATALTDIDLQLQFSTSQPEALLLLAAGPADHLLLQLYSGRLQVRLVLGQEELRLQTPAETLLSDSIPHTVVLTVVEGWATLSVDGFLNASSAVPGAPLEVPYGLFVGGTGTLGLPYLRGTSRPLRGCLHAATLNGRSLLRPLTPDVHEGCAEEFSASDDVALGFSGPHSLAAFPAWGTQDEGTLEFTLTTQSRQAPLAFQAGGRRGDFIYVDIFEGHLRAVVEKGQGTVLLHNSVPVADGQPHEVSVHINAHRLEISVDQYPTHTSNRGVLSYLEPRGSLLLGGLDAEASRHLQEHRLGLTPEATNASLLGCMEDLSVNGQRRGLREALLTRNMAAGCRLEEEEYEDDAYGHYEAFSTLAPEAWPAMELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDPPHIIFPHGSLMVILEHTQKPLGPEVFQAYDPDSACEGLTFQVLGTSSGLPVERRDQPGEPATEFSCRELEAGSLVYVHRGGPAQDLTFRVSDGLQASPPATLKVVAIRPAIQIHRSTGLRLAQGSAMPILPANLSVETNAVGQDVSVLFRVTGALQFGELQKQGAGGVEGAEWWATQAFHQRDVEQGRVRYLSTDPQHHAYDTVENLALEVQVGQEILSNLSFPVTIQRATVWMLRLEPLHTQNTQQETLTTAHLEATLEEAGPSPPTFHYEVVQAPRKGNLQLQGTRLSDGQGFTQDDIQAGRVTYGATARASEAVEDTFRFRVTAPPYFSPLYTFPIHIGGDPDAPVLTNVLLVVPEGGEGVLSADHLFVKSLNSASYLYEVMERPRHGRLAWRGTQDKTTMVTSFTNEDLLRGRLVYQHDDSETTEDDIPFVATRQGESSGDMAWEEVRGVFRVAIQPVNDHAPVQTISRIFHVARGGRRLLTTDDVAFSDADSGFADAQLVLTRKDLLFGSIVAVDEPTRPIYRFTQEDLRKRRVLFVHSGADRGWIQLQVSDGQHQATALLEVQASEPYLRVANGSSLVVPQGGQGTIDTAVLHLDTNLDIRSGDEVHYHVTAGPRWGQLVRAGQPATAFSQQDLLDGAVLYSHNGSLSPRDTMAFSVEAGPVHTDATLQVTIALEGPLAPLKLVRHKKIYVFQGEAAEIRRDQLEAAQEAVPPADIVFSVKSPPSAGYLVMVSRGALADEPPSLDPVQSFSQEAVDTGRVLYLHSRPEAWSDAFSLDVASGLGAPLEGVLVELEVLPAAIPLEAQNFSVPEGGSLTLAPPLLRVSGPYFPTLLGLSLQVLEPPQHGALQKEDGPQARTLSAFSWRMVEEQLIRYVHDGSETLTDSFVLMANASEMDRQSHPVAFTVTVLPVNDQPPILTTNTGLQMWEGATAPIPAEALRSTDGDSGSEDLVYTIEQPSNGRVVLRGAPGTEVRSFTQAQLDGGLVLFSHRGTLDGGFRFRLSDGEHTSPGHFFRVTAQKQVLLSLKGSQTLTVCPGSVQPLSSQTLRASSSAGTDPQLLLYRVVRGPQLGRLFHAQQDSTGEALVNFTQAEVYAGNILYEHEMPPEPFWEAHDTLELQLSSPPARDVAATLAVAVSFEAACPQRPSHLWKNKGLWVPEGQRARITVAALDASNLLASVPSPQRSEHDVLFQVTQFPSRGQLLVSEEPLHAGQPHFLQSQLAAGQLVYAHGGGGTQQDGFHFRAHLQGPAGASVAGPQTSEAFAITVRDVNERPPQPQASVPLRLTRGSRAPISRAQLSVVDPDSAPGEIEYEVQRAPHNGFLSLVGGGLGPVTRFTQADVDSGRLAFVANGSSVAGIFQLSMSDGASPPLPMSLAVDILPSAIEVQLRAPLEVPQALGRSSLSQQQLRVVSDREEPEAAYRLIQGPQYGHLLVGGRPTSAFSQFQIDQGEVVFAFTNFSSSHDHFRVLALARGVNASAVVNVTVRALLHVWAGGPWPQGATLRLDPTVLDAGELANRTGSVPRFRLLEGPRHGRVVRVPRARTEPGGSQLVEQFTQQDLEDGRLGLEVGRPEGRAPGPAGDSLTLELWAQGVPPAVASLDFATEPYNAARPYSVALLSVPEAARTEAGKPESSTPTGEPGPMASSPEPAVAKGGFLSFLEANMFSVIIPMCLVLLLLALILPLLFYLRKRNKTGKHDVQVLTAKPRNGLAGDTETFRKVEPGQAIPLTAVPGQGPPPGGQPDPELLQFCRTPNPALKNGQYWV

SEQ ID NO: 8 - protein, 127 aa, Artificial Sequence
  Humanized VHH 5D nanobody
QVQLVESGGGLVQPGGSLRLSCAASGFTLDYYGIGWFRQAPGQGLEAVSYISASARTILYADSVKGRFTISRDNSKNTLYLQMNSLRAEDTAVYYCARRRFSASSVNRWLADDYDVWGQGTLVTVSS

SEQ ID NO: 9 - protein, 20 aa, Homo sapiens
MYRMQLLSCIALSLALVTNS

SEQ ID NO: 10 - protein, 20 aa, Homo sapiens
METDTLLLWVLLLWVPGSTG

SEQ ID NO: 11 - DNA, 39 bp, Artificial Sequence
  Forward Primer TcdBcore
gggaattccatatgtctgcttggtctcacccacaattcg

SEQ ID NO: 12 - DNA, 35 bp, Artificial Sequence
  Reverse Primer TcdBcore
ccgctcgagtttgaaagccttgccggtttccgggc

SEQ ID NO: 13 - DNA, 37 bp, Artificial Sequence
  Forward Primer Full Length TcdB1
catgccatgggctctgcttggtctcacccacaattcg

SEQ ID NO: 14 - DNA, 35 bp, Artificial Sequence
  Reverse Primer Full length TcdB1
ccgctcgagttagtgatgatgatgatgatgttcag

SEQ ID NO: 15 - DNA, 34 bp, Artificial Sequence
  Forward Primer CSPG4mini
cgcggatccgcttccttcttcggtgagaaccacc

SEQ ID NO: 16 - DNA, 40 bp, Artificial Sequence
  Revese Primer CSPG4mini
ttcttggaccggttactgcacctccagggccaggttctcc

SEQ ID NO: 17 - DNA, 34 bp, Artificial Sequence
  Forward Primer Repeat1-Fc
cgcggatccgagctgcctgagccatgcgtgcctg

SEQ ID NO: 18 - DNA, 35 bp, Artificial Sequence
  Reverse Primer Repeat1-Fc
ctagtctagagtcattgacagggttgacctggatg

SEQ ID NO: 19 - DNA, 34 bp, Artificial Sequence
  Forward Primer CSPG4-ECD
ccggaattcgcttccttcttcggtgagaaccacc

SEQ ID NO: 20 - DNA, 35 bp, Artificial Sequence
  Reverse Primer CSPG4-ECD
ctagtctagagctggatgccatggggcctggctcg

SEQ ID NO: 21 - DNA, 34 bp, Artificial Sequence; identical to SEQ ID NO: 17
  Forward Primer His-Repeat1

SEQ ID NO: 22 - DNA, 38 bp, Artificial Sequence
  Reverse Primer His-Repeat1
ctagtctagactagtcattgacagggttgacctggatg

SEQ ID NO: 23 - DNA, 34 bp, Artificial Sequence
  Forward Primer Bezlo LC
aaaaggcctgagattgtgctgacacagtcccccg

SEQ ID NO: 24 - DNA, 41 bp, Artificial Sequence
  Reverse Primer Bezlo LC
ctagtctagattagcattctcctctattgaagctcttggtc

SEQ ID NO: 25 - DNA, 34 bp, Artificial Sequence
  Forward Primer Bezlo HC
aaaaggcctgaggtgcagctcgtgcagagcggcg

SEQ ID NO: 26 - DNA, 38 bp, Artificial Sequence
  Reverse Primer Bezlo HC
ctagtctagagcaggacttgggctccaccctcttatcg

SEQ ID NO: 27 - DNA, 34 bp, Artificial Sequence; identical to SEQ ID NO: 25
  Forward Primer Belzo VH-CH1

SEQ ID NO: 28 - DNA, 41 bp, Artificial Sequence
  Reverse Primer Bezlo VH-CH1
ctagtctagattagcaggacttgggctccaccctcttatcg

SEQ ID NO: 29 - protein, 10 aa, Homo sapiens
  MOD_RES (7)..(7): Alkene moiety
IPSIISDRPK

SEQ ID NO: 30 - protein, 14 aa, Homo sapiens
  MOD_RES (10)..(10): unsaturated thiol moiety
HVQPTLDLMEAELR

SEQ ID NO: 31 - protein, 21 aa, Homo sapiens
  VARIANT (12)..(12): Ile or Leu
  VARIANT (14)..(14): Asp, Gly, or His
  VARIANT (15)..(15): Val or Ile
  VARIANT (21)..(21): Asn or Lys
LEISSRPYYTNXYXXLYFIMX

SEQ ID NO: 32 - protein, 2366 aa, Clostridium difficile; identical to SEQ ID NO: 1

SEQ ID NO: 33 - protein, 2366 aa, Clostridium difficile
MSLVNRKQLEKMANVRFRVQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFYRKRMEIIYDKQKNFINYYKTQREENPDLIIDDIVKIYLSNEYSKDIDELNSYIEESLNKVTENSGNDVRNFEEFKGGESFKLYEQELVERWNLAAASDILRISALKEVGGVYLDVDMLPGIQPDLFESIEKPSSVTVDFWEMVKLEAIMKYKEYIPGYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKKNYFEGSLGEDDNLDFSQNTVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIDTQVVEGRIEEAKSLTSDSINYIKNEFKLIESISDALYDLKQQNELEESHFISFEDILETDEGFSIRFIDKETGESIFVETEKAIFSEYANHITEEISKIKGTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELILRDKATKVVDYFSHISLAESEGAFTSLDDKIMMPQDDLVISEIDFNNNSITLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPVITTEYIREKLSYSFYGSGGTYALSLSQYNMNINIELNENDTWVIDVDNVVRDVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDKGKENGFINCSTKEGLFVSELSDVVLISKVYMDNSKPLFGYCSNDLKDVKVITKDDVIILTGYYLKDDIKISLSFTIQDENTIKLNGVYLDENGVAEILKFMNKKGSTNTSDSLMSFLESMNIKSIFINSLQSNTKLILDTNFIISGTTSIGQFEFICDKDNNIQPYFIKFNTLETKYTLYVGNRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKVISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDEDTAEAYIGISIINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 34 - protein, 1440 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESAINDTLESFRENLNNPRFDYNKFFRKRMEIIYDKQKNFINYYKAQREENPELIIDDIVKIYLSNEYSKEIDELNTYIEESLNKIKQNSGNDVRNFEEFKNGESFKLYEQELVERWNLAAASDILRISALKEIGGMYLDVDMLPGIQPDLFESIEKPSSVTVDFWEMTKLEAIMKYKEYIPGYTSEHFDMLDEEVQSSFESALASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTVNLSGPEAYAAAYQDLLMFKEGSMNIHLIEADLRNFEISKTNISQSTEQEMASLWTFDDARAKVQFEEYKRNYFEGSLGEDDNLDFSQNIVVDKEYLLEKISSLARSSERGYIHYIVQLQGDKISYEAACNLFAKTPYDSILFQKNIENSEVAYYYNPGDGEIQEIDKYRIPSIISDRPKIKLTFIGHGKDEFNTDIFAGLDVDSLSTEIETAIDLAKEDISSKSIEINLLGCNMFSYSINVEETYPGKLLLKVKDKISELMPSISQDSIIVSANQYEVRINNEGRRELLDHSGEWINKEESIIKDISSKEYISFNPKENKITVKSKNLPELSTLLQEIRNNSNLSDIELEEKVMLAECEINVISNIDTQIVEERIEEAKNLTSDSINYIKNEFKLIESISDSLYDLKQQNELDDSHFISFEDISKTEDGFSIRFINKETGESIFVETEKEIFSEYANHIEREISNIKDTIFDTVNGKLVKKVNLDAIHEVNTLNAAFFIQSLIGYSSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELVLRDKATKVVDYFKHISLVETEGAFTLLDDKIMIPQDDLVISEIDFNNNSIVLGKCEIWRMEGGSGHTVTNDIDHFFSSPTITYIKPHLSIYDVLEVQKEELDLSKDLMVLPNAPNRVFAWETGWTPGLRSLENEGTKLLDRIRDHYKGEFYWRYFAFIADALITTLKPRYEDTNIRINLDSNNRSFIVPIITTEHIREKLSYSFHGSGGTYALSLSQYNMGINIELSESDVWIIDVDNVVRDVTIDSDKIKKGDLIEGILSTLSIEDNKIILNHHEINFSGDVNGSNGFISLTFSILEGINAIIEVDLLSKSYKLLISGELKILMLNS

SEQ ID NO: 35 - protein, 1280 aa, Clostridium difficile
MSLVNRKQLEKMANVRFRVQEDEYVAILDALEEYHNMSENTVVEKYLKLKDINSLTDTYIDTYKKSGRNKALKKFKEYLVIEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKHKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKSSERGYVHYIVQLQGDKISYEAACNLFAKNPYDSILFQKNIEDSEVAYYYNPTDSEIQEIDKYRIPDRISDRPKIKLTFIGHGKAEFNTDIFAGLDVDSLSSEIETAIGLAKEDISPKSIEINLLGCNMFSYSVNVEETYPGKLLLRVKDKVSELMPSMSQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEERIEEAKSLTSDSINYIKNEFKLIESISDALCDLKQQNELEDSHFISFEDISETDEGFSIRFINKETGESIFVETEKTIFSEYANHITEEISKIKGTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPIIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKVMMPQDDLVISEIDFNNNSIVLGKCEIWRMEGGSGHTVTDDIDHFFSAPSITYREPHLSIYDVLEVQKEELDLSKDLMVLPNAPNRVFAWETGWTPGLRSLENDGTKLLDRIRDNYEGEFYWRYFAFIADALITTLK

SEQ ID NO: 36 - protein, 1280 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFFRKRMEIIYDKQKNFINYYKAQREENPELIIDDIVKTYLSNEYSKEIDELNAYIEESLNKITQNSGNDVRNFEEFKNGESFNLYEQELVERWNLAAASDILRISALKEIGGVYLDVDMLPGIQPDLFESIEKPSSVTVDFWEMTKLEAIMKYKEYIPGYTSEHFDMLDEEVQSSFESALASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEDSMNIHLIEADLRNFEIPKTNISQSTEQEMASLWSFDDARAKAQFEEYKRNYFEGSLGEDDNLDFSQNIVVDKEYLLEKISSLARSSERGYIHYIVQLQGDKISYEAACNLFAKTPYDSILFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNSSDIELEEKVMLTECEINVISNIDTQIVEERIEEAKNLTSDSINYIKNEFKLIESISDALCDLKQQNELDDSHFISFEDISETDEGFSIRFINKETGESIFVETEKTIFSEYANHITEEISKIKDTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTITDDIDHFFSAPSITYREPHLSIYDVLEVQKEELDLSKDLMVLPNAPNRVFAWETGWTPGLRSLENDGTKLLDRIRDHYEGEFYWRYFAFIADALITTLKP

SEQ ID NO: 37 - protein, 2367 aa, Clostridium difficile
MSLVNRKQLEKMANVRFRVQEDEYVAILDALEEYHNMSENTVVEKYLKLKDINSLTDTYIDTYKKSGRNKALKKFKEYLVTEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKYKEYIPEYTSKHFDTLDEEVQSSFESVLASKSDKSEIFLPLGDIEVSPLEVKIAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVIDKEYLLEKISSSTKSSERGYVHYIVQLQGDKISYEAACNLFAKNPYDSILFQKNIEDSEVAYYYNPTDSEIQEIDKYRIPDRISDRPKIKLTLIGHGKAEFNTDIFAGLDVDSLSSEIETIIDLAKADISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEERIEEAKSLTSDSINYIKNEFKLIESISDALYDLKQQNELEESHFISFEDISETDEGFSIRFIDKETGESIFVETEKAIFSEYANHITEEISKIKGTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELILRDKATKVVDYFSHISLAESEGAFTSLDDKIMMPQDDLVISEIDFNNNSITLGKCEIWRMEGGSGHTVTDDIDHFFSSPSITYREPHLSIYDVLEVKKEELDLSKDLMVLPNAPNRVFGWETGWTPGLRGLENDGTKLLDRIRDQYEGQFYWRFFAFIADALITTLKPRYEDTNVRISLDSNTRSFIVPVITTEYIREKLSYSFYGSGGTYALSLSQYNMNINIELNENDTWVIDVDNVVRSVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDKGKENGFINCSTKEGLFVSELSDVVLIIKVYMDNSKPPFGYYSNDLKDVKAITKDDVIILTGYYLKDDIKISLSFTIQDKNTIKLNGVYLDENGVAEILKFMNKKGSTNTSDSLMSFLESMNIKSIFIKSLKSNAKLILDTNFIISGTTSIGQFEFICDKDNNIQPYFIKFNTLETKYTLYVGNRQNMIVEPNYDLDDSGDISSTVINFSQKYLYGIDSCVNKVIISPNIYTDEINITPIYEANNTYPEVIVLDTNYISEKINININDLSIRYVWSNDGSDFILMSTDEENKVSQVKIRFTNVFKGNTISDKISFNFSDKQDVSINKIISTFTPSYYVEGLLNYHLGLISLYNEKFYINNFGMMVSGLVYINDSLYYFKPPIKNLITGFTTIGDDKYYFNPDNGGAASVGETIIDGKNYYFSPNGVLQTGVFSTEDGFKYFAPADTLDENLEGEAIDFTGKLIIDENVYYFGDNYRAAIEWQTLDDEVYYFSTDTGRAFKGLNQIGDDKFYFNSDGIMQKGFVNINDKTFYFDDSGVMKSGYTEIDGKHFYFAENGEMQIGVFNTADGFKYFAHHDEDLGNEEGEALSYSGILNFNNKIYYFDDSFTAVVGWKDSEDGSKYYFDEDTAEAYIGISTINDGKYYFNDSGIMQIGFVTINNEVFYFSDSGIVESGMQNIDDNYFYIDENGLVQIGVFDTSDGYKYFAPANTVNDNIYGQAVEYSGLVRVGEDVYYFGETYTIETGWIYDMENESDKYYFDPETKKAYKGINVIDDIKYYFDENGIMRTGLITFEDNHYYFNEDGIMQYGYINIEDKMFYFNEDGVMQIGVFNTADGFKYFAHQNTLDENFEGESINYTGWLDLDEKRYYFTDEYIAAIGSVIIDGEEYYFDPDTAELVISE

SEQ ID NO: 38 - protein, 2367 aa, Clostridium difficile
MSLVNRKQLEKMANVRFRVQEDEYVAILDALEEYHNMSENTVVEKYLKLKDINSLTDTYIDTYKKSGRNKALKKFKEYLVTEILELKNSNLTPVEKNLHFIWIGGQINDTAINYINQWKDVNSDYNVNVFYDSNAFLINTLKKTIIESASNDTLESFRENLNDPEFNHTAFFRKRMQIIYDKQQNFINYYKAQKEENPDLIIDDIVKTYLSNEYSKDIDELNAYIEESLNKVTENSGNDVRNFEEFKTGEVFNLYEQELVERWNLAGASDILRVAILKNIGGVYLDVDMLPGIHPDLFKDINKPDSVKTAVDWEEMQLEAIMKYKEYIPEYTSKHFDTLDEEVQSSFESVLASKSDKSEIFLPLGDIEVSPLEVKVAFAKGSIINQALISAKDSYCSDLLIKQIQNRYKILNDTLGPIISQGNDFNTTMNNFGESLGAIANEENISFIAKIGSYLRVGFYPEANTTITLSGPTIYAGAYKDLLTFKEMSIDTSILSSELRNFEFPKVNISQATEQEKNSLWQFNEERAKIQFEEYKKNYFEGALGEDDNLDFSQNTVTDKEYLLEKISSSTKSSERGYVHYIVQLQGDKISYEAACNLFAKNPYDSILFQKNIEDSEVAYYYNPTDSEIQEIDKYRIPDRISDRPKIKLTLIGHGKAEFNTDIFAGLDVDSLSSEIETIIDLAKADISPKSIEINLLGCNMFSYSVNVEETYPGKLLLRVKDKVSELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIETQVVEERIEEAKSLTSDSINYIKNEFKLIESISDALYDLKQQNELEESHFISFEDISKTDEGFSIRFIDKETGESIFVETEKAIFSEYANHITEEISKLKDTIFDTVNGKLVKKVTLDATHEVNTLNAAFFIQSLIGYNSSKESLSNLSVAMKVQVYAQLFSTGLNTITDAAKVVELVSTALDETIDLLPTLSEGLPVIATIIDGVSLGASIKELSETSDPLLRQEIEAKIGIMAVNLTAATTAIITSSLGIASGFSILLVPLAGISAGIPSLVNNELILRAEAKNVVDYFGHISLAESEGAFTLLDDKIMMPQDDLVISEIDFNNNSITLGKCEIWRMEGGSGHTVTDDIDHFFSAPSTTYREPYLSIYDVLDVKKEELDLSKDLMVLPNAPDRIFGWERGWTPGLRSLENDGTKLLDRIRDHYEGQFYWRFFAFIADSVITKLKPRYEDTNIRISLDSNTRSFIVPVITTEYIREKLSYSFYGSGGTYALSLSQYNMNINIELNENDTWVIDVDNVVRDVTIESDKIKKGDLIENILSKLSIEDNKIILDNHEINFSGTLNGGNGFVSLTFSILEGINAVIEVDLLSKSYKVLISGELKTLMANSNSVQQKIDYIGLNSELQKNIPYSFMDDEGKENGFINCFTKEGLFVSELSDVVLIIKVYMDNSKPPFGYYSNDLKDVKVITKDDVIILTGYYLKDDIKISLSFTIQDKNTIKLNGVYLDENGVAEILKFMNKKGSTNTSDSLMSFLESMNIKSIFIKSLKSNAKLILDTNFIISGTTSIGQFEFICDKDNNIQPYFIKFNTLETKYTLYVGNRQNMIVEPNYNLDDSGDISSTVINFSQKYLYGIDSCVNKVIISPNIYTDEINITPVHEANNTYPEVIVLDTNYISEKINININDLSIRYVWSNDGSDFILMSTDEENKVSQVKIRFTNVFKGNTISDKISFNFSDKQDISINKIISTFTPSYYVEGLLNYDLGLISLYNEKFYINNLGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLIIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDENTAEASIGISIINDGKYYFNDSGIMQIGFVTINNEVFYFSDSGIVESGMQNIDDNYFYISENGLVQIGVFDTSDGYKYFAPANTVNDNIYGQAVEYSGLVRVNEDVYSFGESYTIETGWIYDSENESDKYYFDPETKKAYKGINVIDDIKYYFDENGIMRTGLITFEDNHYYFNEDGEMQYGYLNIEDKMFYFSEDGIMQIGVFNTPDGFKYFAHQNTLDENFEGESINYTGWLDLDEKRYYFTDEYIAATGSVIIDGEEYYFDPDTAQLVISE

SEQ ID NO: 39 - protein, 2367 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPEFNHTAFFRKRMQIIYDKQQNFINYYKAQKEENPDLIIDDTVKSYLSDEYSKDIDELNAYIEESLNKIAENSGNDVRNFEEFKDGEVFNLYEQELVERWNLAAASDILRVAILKNIGGVYLDVDMLPGIHPDLFKNINKPDSVKTAVDWEEMKLEAIMKYKEYIPEYTSKHFDTLDEEVQSSFESVLASKSDKSEIFLPLGDIEVSPLEVKIAFAKGSIINQALISVKDSYCSDLLIKQIQNRYKILNDTLGPIISQGNDFNTTMNSFGESLGAISSEDNISFIAKIGSYLRVGFYPEANTTITLSGPTVYAGAYKDLLTFKEISLDTSILTSELRNFEFPKDNISQATEQEKNSLWQFNEERAKIQFEEYKRAYFEGALGEDDNLDFSQNTVTDKEYLLEKISSSTKSSERGYVHYIVQLQGDKISYEAACNLFAKNPYDSILFQKNIEDSEIAYYYNPADGEIQEIDKYRIPDRISDRPKIKLTFIGHGKDEFNTDIFAGLDVDSLSTEIETAIDLAKEDISSKSIEINLLGCNMFSYSINVEETYPGKLLLKVKDKISELMPSISQDSIIVSANQYEVRINSEGRRELLDHSGEWINKEESIIKDISSKEYISFNPKENKITVKSKNLPELSTLLQEIRNNSNLSDIELEEKVMLAECEINVISNIDTQIVEERIEEAKNLTSDSINYIKNEFKLIESISDSLYDLKQQNELDDSHFISFEDISKTEDGFSIRFINKETGESIFVETEKEIFSEYANHIEREISNIKDTIFDTVNGKLVKKVNLDAIHEVNTLNAAFFIQSLIGYSSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTAATTAIITSSLGIASGFSILLVPLAGISAGIPSLVNNELVLRDKATKVVDYFKHISLVETEGAFTLLDDKIMIPQDDLVISEIDFNNNSIVLGKCEIWRMEGGSGHTVTNDIDHFFSSPTITYIKPHLSIYDVLEVQKEELDLSKDLMVLPNAPNRVFAWETGWTPGLRSLENEGTKLLDRIRDHYKGEFYWRYFAFIADALITTLKPRYEDTNIRINLDSNNRSFIVPIITTEHIREKLSYSFHGSGGTYALSLSQYNMGINIELSESDVWIIDVDNVVRDVTIDSDKIKKGDLIEGILSTLSIEDNKIILNHHEINFSGDVNGSNGFISLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTGYYLKDDIKISLSFTLQDEKTIKLNGVHLDESGVAEILKFMNKKGSTNTSDSLMSFLESVNIKSIFVNFLQSKINFILDANFIISGTTSIGQFEFICDENDNIQPYFIKFNTLETTYTLYVGNRQNMIVEPNYDLDDSGDISSTVINFSQKYLYGIDSCVNKVVISPNIYTDEINITPVYETNNNYPEVIVLDANYINEKINVNINDLSIRYVWSNDGNDFILMSTSEENKVSQVKIRFVNVFKDKTLANKLSFNFSDKQDVPVSEIISAFTPSYYEDGLIGYDLGLVSLYNEKFYINNFGMMVSGLIYINDSLYYFKPPVNNLITGFVTVGDDKYYFNPTNGGAASIGETIIDDKNYYFNQSGILQTGVFSTEDGLKYFAPANTLDENLEGEAIDFTGKLIIDENIYYFEDNYRGAVEWKELDGEMYYFSPETGKAFKGLNQIGDDKYYFNSDGIMKKGFVSINDKKYYFDDSGVMKVGYIEIDGKYFYFAENGEMQIGVFNTSDGFKYFAHHNEDLGNEEGEAISYSGILNFNNKIYYFDYSFTAVVGWKDLEDGSKYYFDEDTAEAYVGLSLINDGQYYFNDDGIMQVGFVTINNKVFYFSDSGIIESGVQNIDDNYFYIDEKGIVQIGVFDTSDGYKYFAPANTVNDNIYGQAVDYSGLVRVGEDIYYFGETYTIETGWIYDMENESDKYYFNPETKKACKGINLIDDIKYYFDENGIMRTGLISFENNDYYFNENGEMHFGYINIEDKMFYFGEDGVMQIGVFNTPDGFKYFAHQNTLDENFEGESINYTGWLDLEGKRYYFTDEYIAATGTVTIDGEEYYFDPDTAELVVSE

SEQ ID NO: 40 - protein, 2366 aa, Clostridium difficile
MSLVNRKQLEKMANVRFRTQEDEYVAILDALEEYHNMSENTVVEKYLKLKDINSLTDIYIDTYKKSGRNKALKKFKEYLVTEVLELKNNNLTPVEKNLHFVWIGGQINDTAINYINQWKDVNSDYNVNVFYDSNAFLINTLKKTIVESATNDTLESFRENLNDPRFDYNKFYRKRMEIIYDKQKNFINYYKTQREENPDLIIDDIVKIYLSNEYSKDIDELNSYIEESLNKVTENSGNDVRNFEEFKGGESFKLYEQELVERWNLAAASDILRISALKEVGGVYLDVDMLPGIQPDLFESIEKPSSVTVDFWEMVKLEAIMKYKEYIPGYTSEHFDMLDEEVQSSFESVLASKSDKSEIFSSLGDMEASPLEVKIAFNSKGIINQGLISVKDSYCSNLIVKQIENRYKILNNSLNPAISEDNDFNTTTNAFIDSIMAEANADNGRFMMELGKYLRVGFFPDVKTTINLSGPEAYAAAYQDLLMFKEGSMNIHLIEADLRNFEISKTNISQSTEQEMASLWSFDDARAKAQFEEYKKNYFEGSLGEDDNLDFSQNTVVDKEYLLEKISSLARSSERGYIHYIVQLQGDKISYEAACNLFAKTPYDSVLFQKNIEDSEIAYYYNPGDGEIQEIDKYKIPSIISDRPKIKLTFIGHGKDEFNTDIFAGLDVDSLSTEIETAIDLAKEDISPKSIEINLLGCNMFSYSINVEETYPGKLLLKVKDKISELMPSISQDSIIVSANQYEVRINSEGRRELLDHSGEWINKEESIIKDISSKEYISFNPKENKIIVKSKNLPELSTLLQEIRNNSNSSDIELEEKVMLAECEINVISNIDTQVVEERIEEAKSLTSDSINYIKNEFKLIESISDALYDLKQQNELEESHFISFEDISETDEGFSIRFIDKETGESIFVETEKAIFSEYANHITEEISKLKDTIFDTVNGKLVKKVNLDATHEVNTLNAAFFIQSLIEYNSSKESLSNLSVAMKVQVYAQLFSTGLNTITDAAKVVELVSTALDETIDLLPTLSEGLPVIATIIDGVSLGAAIKELSETSDPLLRQEIEAKIGIMAVNLTTATTAIITSSLGIASGFSILLVPLAGISAGIPSLVNNELVLRDKATKVVDYFKHVSLVETEGVFTLLDDKIMMPQDDLVISEIDFNNNSIVLGKCEIWRMEGGSGHTITDDIDHFFSAPSITYREPHLSIYDVLEVQKEELDLSKDLMVLPNAPNRVFAWETGWTPGLRSLENDGTKLLDRIRDNYEGEFYWRYFAFIADALITTLKPRYEDTNIRINLDSNTRSFIVPIITTEYIREKLSYSFYGSGGTYALSLSQYNMGINIELSESDVWIIDVDNVVRDVTIESDKIKKGDLIEGILSTLSIEENKIILNSHEINFSGDVNGSNGFVSLTFSILEGINAIIEVDLLSKSYKLLISGELKILMLNSNHIQQKIDYIGFNSELQKNIPYSFVDSEGKENGFINGSTKEGLFVSELPDVVLISKVYMDDSKPSFGYYSNNLKDVKVITKDNVNILTSYYLKDDIKISLSLTLQDEKTIKLNSVHLDESGVAEILKFMNRKGSTNTSDSLMSFLESMNIKSIFVNFLQSNIKFILDANFIISGTTSIGQFEFICDENDNIQPYFIKFNTLETNYTLYVGNRQNMIVEPNYDLDDSGDISSTVINFSQKYLYGIDSCVNKVVISPNIYTDEINITPVYEANNTYPEVIVLDTNYISEKINININDLSIRYVWSNDGSDFILMSTDEENKVSQVKIRFTNVFKGNTISDKISFNFSDKQDVSINKIISTFTPSYYVEGLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAIDFTGKLTIDENVYYFGDNYRAAIEWQTLDDEVYYFSTDTGRAFKGLNQIGDDKFYFNSDGIMQKGFVNINDKTFYFDDSGVMKSGYTEIDGKYFYFAENGEMQIGVFNTADGFKYFAHHDEDLGNEEGEALSYSGILNFNNKIYYFDDSFTAVVGWKDLEDGSKYYFDENTAEASIGISIINDGKYYFNDSGIMQIGFVTINNEVFYFSDSGIVESGMQNIDDNYFYISENGLVQIGVFDTSDGYKYFAPANTVNDNIYGQAVEYSGLVRVNEDVYSFGESYTIETGWIYDSENESDKYYFDPETKKAYKGINVIDDIKYYFDENGIMRTGLITFEDNHYYFNEDGIMQYGYLNIEDKTFYFSEDGIMQIGVFNTPDGFKYFAHQNTLDENFEGESINYTGWLDLDEKRYYFTDEYIAATGSVIIDGEEYYFDPDTAELVISE

SEQ ID NO: 41 - protein, 2366 aa, Clostridium difficile
MSLVNRKQLEKMANVRFRVQEDEYVAILDALEEYHNMSENTVVEKYLKLKDINSLTDTYIDTYKKSGRNKALKKFKEYLVTEVLELKSSNVVPVEKNLHFVWIGGKINDTAINYINQWKDVNSDYNVNVFYDSNAFLINTLKKTIVDSATNETLESFRENLDDPRFDYNKFYRKRMEIIYDKQKNFINYYKAQREENPDFIIDDIVKSYLSNEYSKDIDELNAYIEESLNKVKENSGNDIRDFEEFKGGNSFNLYEQELVERWNLAAASDILRISALKEVGGVYLDVDMLPGIQPDLFESIEKPSSVTVDFWEMVKLEAIMKYKEYIPGYTSEHFDILDEEVQSSFESVLASKSDKSEIFSSLGDIEASPLEVKIAFNSKGIINQGLISVKDSYCSNLIVKQIENRYKILNDSLNPAISEDNDFNTTTNTFIDSIMAEANADNGRFMMELGKYLRVGFFPDVKTTINLSGPEAYAAAYQDLLMFKEYSINIHLLESDLRNFEISKTNISQSTEQEMASLWSFDDARAKAQFQEYKRNYFEGALGEDDNLDFSQNTITDKEYLIEKISSSAKNSERGYVHYIIQLQGDNISYEAACNLFAKNPYDSILFQKNIEDSTIAYYYNPADGEIQEIDKYRIPDRISDRPKIKLTFIGHGKSEFNTDIFANLNVDSLSSEIETAIDLAKTDISPKAIEINLLGCNMFSYSVNVEETYPGKLLLKVKDKVSELMPSISQDSIIVSANQYEVRINSEGRRELLDHSGEWINKEESIIKDISSKEYISFNSKENKIIVKSKNLPELSTLLQEIRNNSNLSDIELEEKVMLAECEISVVSDIDTQVVEERIEEAKNLTSDSINYIKNEFKLIESISDALYDLKQQNELEDSHFISFEDISETDEGFSIRFIDKETGESIFVETEKTIFSEYANHITEEISKVKDTIFDTVNGKLVKKVNLDATHEVNTLNAAFFIQSLIGYNSSKESLSNLSVAMKVQVYAQLFSTGLNTITDAAKVVELVSTALDETIDLLPTLSEGLPIIATIIDGVSLGASIKELSETSDPLLRQEIEAKIGIMAVNLTAATTAIITSSLGIASGFSILLVPLAGISAGIPSLVNNELILRAEAKNVVDYFSHISLAESEGAFTLLDDKIMMPQDDLVISEIDFNSNSITLGKCEIWRMEGGSGHTVTNDIDHFFSAPSTTYREPYLSIYDVLDVKKEELDLSKDLMVLPNAPDRIFGWERGWTPGLRGLENDGTKLLDRIRDHYEGQFYWRFFAFIADSVITKLKPRYEDTNIRISLDSNTRSFIVPVITTEYIREKLSYSFYGSGGTYALSLSQYNMNINIELNESDTWVIDIDNVVRDVTIESDKIKKGDLIENILSKLSIEENKIILDNHEINFSGTLNGGNGFVSLTFSILEGINAVIEVDLLSKSYKVLISGELKTLMENSNSVQQKMDYIGLNSEVQKNIPYSFTDDKGKENGFINCSTKEGLFVSELSDVVLISKVYMDDSKPSSGYYSYDLKDVKVITKDDVIILTGYYLKDDIKISLSFTIQDENTIKLNGVYLDENGVAEILKFMNKKGSTNTSDSLMSFLESMNIKSIFINSLQSNTKLILDTNFIISGATSIGQFEFICDKDNNIQPYFIKFNTLETKYTLYVGNRQNMIVEPNYNLDDSGDISSTVINFSQKYLYGIDSCVNKVIISPNIYTDEINITPVYEANNTYPEVIVLDTNYISEKINININDLSIRYVWSNDGSDFILMSTDEENKISQVKIRFTNVFKGNTIADKISFNFSDKQDVSINKIISTFTPSYYVEQLLNYDLGLISLYNEKFYINNFGMMVSGLVYINDSLYYFKPPIKNLITGFTTIGDDKYYFNPDNGGAASVGETIIDGKNYYFSQNGVLQTGVFSTEDGFKYFAPADTLDENLEGEAINFTGKLIIDENIYYFGDNYRAAEEWQTLDDEVYYFSTDTGKAFKGLNQIGDDKFYFNSDGIMQKGFVNINDKTFYFDDSGVMKSGYLEIYGKYFYFAENGEMQIGVFNTTDGFKYFAHQDEDLGNEEGEALSYSGILNFNNKIYYFDDSFTAIVGWKDLEDGSKYYFDENTAEASIGISIINDGKYYFNDSGIMQIGFVTINDKVFYFSDSGIVESGMQNIDDNYFYISENGLVQIGVFDTSDGYKYFAPANTVNDNIYGQAVEYSGLVKVNEDVYSFGESYTIETGWIYDSENESDKYYFDPETKKAYKGINTIDDIKYYFDENGIMRTGLITFEDNHYYFNEDGVMQYGYLNIEDKMFYFNEDGVMQIGVFNTPDGFKYFAHQNTLDENFEGESINYTGWLDLDGKKYYFTEEYIAATGSVTIDDEEYYFDPDTAELVVSE

SEQ ID NO: 42 - protein, 2366 aa, Clostridium difficile
MSLINRKQLEKMANVKFRVQEDEYIAILDALEEYHNMSENTVVEKYLKLKDINSLTETYIDTYKKSGRNKALKKFKEYLVTEVLELKNSNVAPVEKNLHFVWIGGKINDTAINYINQWKDVNSDYNVNVFYDSNAFLINTLKKTIVDSATNETLESFRENLDDPRFDYNKFYRKRMEIIYDKQKNFINYYKAQREENPDLIIDDIVKTYLSNEYSKDIDELNAYIEESLSKVTENSGNDVRNFEEFKGGESFNLYEQELVERWNLAAASDILRVSALKEVGGVYLDVDMLPGIQPDLFESIEKPSSVTVDFWEMVKLEAIMKYKEYIPGYTSEHFDMLDEEVQSSFESVLASKSDKSEIFSSLGDVESSPLEVKIAFNSKGIINQGLISVKDSYCSNLIVKQIENRYKILNNSLNPAISEDNDFNTTTNTFIDSIMAEANADNSRFMMELGKYLRVGFFPDVKTTINLSGPEAYAAAYQDLLMFKEYSMNIHLLESDLRNFEISKTNISQSTEQEMASLWSFDDARAKAQFQEYKKNYFEGALGEDDNLDFSENTVLDKDYILEKISSSTRSSERGYVHYIVQLQGDKISYEAACNLFAKNPYDSILFQNNIEDSEIAYYYNPADGEIQEIDKYRIPDIISDRPKVKLTFIGHGKSEFNTDIFANLDVDSLSSEIETVIDLAKTDISPKAIEINLLGCNMFSYSINVEDTYPGKLLLKVKDKVSELLPSINQDSIIVSANQYEVRINSEGRRELLDHSGEWINKEESIIKDISSKEYISFNPQENKIIVKSKNLPELSTLVQEIRNNSNSGDIELEEKVMLAECEISVVSDIDTQVVEERIEEAKNLTSDSINYIKNEFKLIESISDALYDLKQQNELEDSHFISFEDISETDEGFSIRFIDKETGESIFVETEKTIFSEYANHITEEITKVKDTIFDTVNGKLVKKVNLDATHEVNTLNAAFFIQSLIGYNSSKESLSNLSVAMKVQVYAQLFSTGLNTITDAAKVVELVSTALDETIDLLPTLSEGLPIIATIIDGVSLGASIKELSETSDPLLRQEIEAKIGIMAVNLTAATTAIITSALGIASGFSILLVPLAGISAGVPSLVNNELVLRDKATKVVDYFSHISLAESEGAFTLLDDKIMMLQDDLVISEIDFNNNSITLGKCEIWRMEGGSGHTVVDDIDHFFSSPPITYREPHLSIYDVLEVKKEELDLSKDLMVLPNAPNRVFGWETGWTPGLRGLENDGTKLLDRIRDYYEGQFYWRFYAFVADALITTLKPRYEDTNVRISLDSNTRSFIVPVITTEYIREKLSYSFYGSGGTYALSLSQYNMNINIELNENDTWVIDVDNVVRDVTIESDKIKKGDLIENILSKLSIEENKIILDNHEINFSGTLNGGNGFVSLTFSILEGINAVIEVDLLSKSYKVLVSGELKTLMLNSNSVQQKIDYIGLNSEVQKNIPYSFTDDKGKENGFINCSTKEGLFISELSDVVLISKVYMDDSKPSFGYYSDDLKDVKVITKDDVILLTGYYLKDDIKISLSFTIQDENTIKLNGVYLDENGVAEILKFMNKKGSTNTSDSLMSFLESMNIKSIFMNFVQSNVKLILDTNFIINGTTSIGQFEFICDKDNNIQPYFIKFNTLETKYTLYVGNRENMIVEPNYNLDDSGDISSTVINFSQKYLYGIDSCINKVIISPNIYTDEINITPVYEANNTYPEVIVLDANYISEKINININDLSIRYVWSNDGSDFILMSTNEEDKVSQIKIRFTNVFKGNTMSDKLSFNFSDKQDVSISKIISTFTPSYYRENLLNYDLGMISLYNEKFYINNFGMMVSGLVYINDSLYYFKPPLKNLITGFTTIGDDKYYFNPDNGGAASVGETIIDGKNYYFSQNGVLQTGVFSTEDGFKYFAPANTLDENLEGEAIDFTGKLTIDENVYYFGDNYRAAIEWQTLDDEMYYFSTETGRAFKGLNQIGDDKFYFNSSGIMQKGFVNINDKTFYFDDSGVMKSGYIEIDGKYFYFAENGEMQIGVFNTTDGFKYFAHQDEDLGNEEGEALSYSGILNFNNKIYYFDDSFTAVVGWKDLEDGSKYYFDEDTAEAYIGISTINDGQYYFNDSGIMQIGFVTINDKVFYFSDSGIVESGMQNIDDNYFYIDDNGLVQIGVFDTSDGYKYFAPANTVNDNIYGQAVECSGLVRVGEDVYCFGESYTIETGWIYDMENESDKYYFDSETKKAYKGINVIDDIKYYFDENGIMRTGLISFENNHYYFNADGEMQYGYLNIEDKMFYFSEDGFMQIGVFNTPDGFKYFAHQSTLDENFEGESINYTGWLDLDDKKYYFTDEYIAATGSVIIDDEEYYFDPETAELVVSE

SEQ ID NO: 43 - protein, 2366 aa, Clostridium difficile
MSLVNRKQLEKMANVKFRTQEDEYVAILDALEEYHNMSENTVVEKYLKLKDINSLTDAYIDTYKKSGRNKALKKFKEYLTTEVIELKNSNLTPVEKNLHFVWIGGQISDTAINYINQWKDVNSDYNTNVFYDSNAFLINTLKKTIVEATTNDTLESFSENLNDPRFDHNNFYRKRMEMIYDKQKNFINYYKAQREENPELIIDDIVKTYLSNEYSKEIDELNAYIEESLNKITQNSGNDVRNFEEFKNGESFKLYEQELVERWNLAAASDILRISALKEIGGVYLDVDMLPGIQPDLFESIEKPSSVTVDFWEMTKLEAIMKYKEYIPGYTSEHFDMLDEEVQSSFESALASKSDKSEIFSSLGDMEASPLEVKIAFNSKGIINQGLISVKDSYCSNLIVKQIENRYKILNNSLNPAISEDNDFNTTTNTFIDSIMAEANADNGRFMMELGKYLRVGFFPDVKTTINLSGPEAYAAAYQDLLMFKEDSMNIHLIEADLRNFEISKTNISQSTEQEMASLWSFDDARAKAQFEEYKRNYFEGALGEDDNLDFSQNTVVDKEYLLEKISSLARSSERGYIHYIVQLQGDKISYEAACNLFAKTPYDSILFQKNIEDSEIAYYYNPGDDEIQEIDKYRIPSIISDRPKIKLTFIGHGKDEFNTDIFAGLDVDSLSTEIETVIDLAKEDISPKAIEINLLGCNMFSYSINIEETYPGKLLLKVKDKISELMPSISQDSIIVSANQYEVRINSEGRRELLDHSGEWINKEESIIKDISSKEYISFNSKENKIIVKSKNLPELSTLLQEIRNNSNSSDIELEEKVMLAECEINVISNIDTQIVEERIEEAKNLTSDSINYIKNEFKLIESISDALCDLKQQNELEDSHFISFEDISETDEGFSIRFINKETGESIFVETEKTIFSEYANHITEEISKVKDTIFDTVNGKLVKKVNLDTTHEVNTLNAAFFIQSLIEYNSSKESLSNLSVAMKVQVYAQLFSTGLNTITDAAKVVELVSTALDETIDLLPTLSEGLPVIATIIDGVSLGSAIKELSETSDPLLRQEIEAKIGIMAVNLTAATTAIITSALGIASGFSILLVPLAGISAGIPSLVNNELILRDKATKVVDYFKHISLAETEGAFTLLDDKIIMPQDDLVISEIDFNNNSITLGKCEIWRMEGGSGHTVTDDIDHFFSSPSITYREPYLSIYDVLEVQKEELDLSKDLMVLPNAPNRVFAWETGWTPGLRSLENDGTKLLDRIRNHYEGEFYWRYFAFIADALITTLKPRYEDTNIRINLDSNTRSFVVPVITTEYIRENLSYSFYGSGGTYALSLSQYNMGINIELSESDVWIIDVDNVVRDVTIESDKIKKGDLIEGILSTLSIEDNKIILNSHELNFSGDVNGSNGFVSLTFSILEGINAIIEVDLLSKSYKLLLSGELKTLMSNSNYIQQKIDYIGFNSELQKNIPYSFVDAEGKKNGFINVSTKEGLFASELSDVVLISKVYMDNSKPSFGHYSDILKDVKVITKDDINILTGYYLKDDIKISLSFTLQDEHTIKLNGVHLDEKGVAEILTFMNKKVGTNTSDSLMSFLKSMNINNVFSHSLQDKVNLVLETNFIISGMTSIGQFEFICDENDNIQPYFIKFNALDTKYTLYLGNRQNMIVEPNYDLDDSGNISSTVINFSQKHLYGIDSFINKVIISPNLYTDEINITPVHETNNTYPEVIVLDANYISEKIKVNINDLSIRYIWSNDGNDFILMSTIGEDKASQVKIRFANVFKGNTLANKLSFNFSDKQDVSLSEIISAFTPSKYEDGFSSYKLGLISFYNEKFYINNFGMKVSGLIYINDSLYYFKPPVNNLITGFTTVGDDKYYFNPTNGGAASIGDTIIDDKNYYFNQIGVLQTGVFSTEDGFKYFAPANTLDENLEGEAIDFTGKLIIDENIYYFEDNYRGAVEWKELDGEMYYFSPETGKAFKGLNQIGDDKYYFNSDGIMQKGFVSINDKKHYFDDSGVMKVGYTEIDGKYFYFAENGEMQIGVFNTSDGFKYFAHYNEDLGNEEGEALSYSGILNFNNKIYYFDDSFTAVVGWRNLDDGSKYYFDENTAEAFIGFSLINDEQYYFNEDGIMQVGFVTINDRVFYFSDSGIIESGVQNIDDNYFYIDEKGIVQIGVFDTSDGYKYFAPPNTVNENIYGQAVEYSGLVKVNEDVYYFGETYLIETGWIYDMENESDKYYFDPETKKAYKGINVINDTKYYFDENGIMRTGLISFENNHYYFNEDGVMQSGYINIEDKMFYFSEDGIMQIGVFNTPDGFKYFAHQNTLDDNFEGESINYTGWLDFNEKRYYFTDEYIAATGSVTIDDEEYYFDPDTAELVLSE

SEQ ID NO: 44 - protein, 20 aa, Artificial Sequence
  sequence conservation of key amino acids consisting of the epitope-1 of bezlotoxumab
  VARIANT (1)..(1): Ile or Val
  VARIANT (11)..(11): Phe or Leu
  VARIANT (12)..(12): Asp, Glu, or Gly
  VARIANT (15)..(15): Ala or Gly
  VARIANT (17)..(17): Val, or Ile
  VARIANT (19)..(19): Pro or Thr
  VARIANT (20)..(20): Glu or Asp
XGEYQSTEDGXXNRXAXEXX

SEQ ID NO: 45 - protein, 20 aa, Artificial Sequence
  sequence conservation of key amino acids consisting of the epitope-2 of bezlotoxumab
  VARIANT (1)..(1): Val or Ser
  VARIANT (8)..(8): Glu, Ala, SEr
  VARIANT (19)..(19): Asp or Asn
XGYYENTXDGFDSTAVVEXT

SEQ ID NO: 46 - protein, 543 aa, Artificial Sequence
  Receptor Decoy Antibody (RDA) composition 1
HHHHHHHHIEGRPASGSLPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDSRTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSVDQYNGERGISIPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKG

SEQ ID NO: 47 - protein, 671 aa, Artificial Sequence
  receptor decoy antibody (RDA) composition-1 with a humanized 5D nanobody
HHHHHHHHIEGRPASGSELPEPCVPEPGLPPVFANFTQLLTISPLVVAEGGTAWLEWRHVQPTLDLMEAELRKSQVLFSVTRGARHGELELDIPGAQARKMFTLLDVVNRKARFIHDGSEDTSDQLVLEVSVTARVPMPSCLRRGQTYLLPIQVNPVNDSRTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSVDQYNGERGISIPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGQVQLVESGGGLVQPGGSLRLSCAASGFTLDYYGIGWFRQAPGQGLEAVSYISASARTILYADSVKGRFTISRDNSKNTLYLQMNSLRAEDTAVYYCARRRFSASSVNRWLADDYDVWGQGTLVTVSS